SEQUENCE LISTING (128 sequences)

SEQ ID NO: 1 - protein, 735 aa, unidentified [sequence omitted]

SEQ ID NO: 2 - protein, 736 aa, unidentified
  source 1..736: note = Dependoparvovirus adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYAKSANVDFTVDNNGVYSEPRPIGTRYLTRNL

SEQ ID NO: 3 - protein, 736 aa, unidentified
  source 1..736: note = Dependoparvovirus adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKVSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYAKSANVDFTVDNNGVYSEPRPIGTRYLTRNL

SEQ ID NO: 4 - protein, 736 aa, unidentified
  source 1..736: note = Dependoparvovirus adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNFAKSANVDFTVDNNGVYSEPRPIGTRFLTRNL

SEQ ID NO: 5 - protein, 736 aa, unidentified
  source 1..736: note = Dependoparvovirus adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSASTGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKVSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNFAKSANVDFTVDNNGVYSEPRPIGTRFLTRNL

SEQ ID NO: 6 - protein, 724 aa, unidentified
  source 1..724: note = Dependoparvovirus adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 7 - protein, 736 aa, unidentified
  source 1..736: note = Dependoparvovirus adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 8 - protein, 736 aa, unidentified
  source 1..736: note = Dependoparvovirus adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, unidentified
  source 1..736: note = Dependoparvovirus adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTVVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, unidentified
  source 1..736: note = Dependoparvovirus adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNFYKSNNVEFAVNTEGVYSEPRPIGTRFLTRNL

SEQ ID NO: 11 - protein, 736 aa, unidentified
  source 1..736: note = Dependoparvovirus adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTVVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNFYKSNNVEFAVNTEGVYSEPRPIGTRFLTRNL

SEQ ID NO: 12 - protein, 743 aa, unidentified
  source 1..743: note = Dependoparvovirus adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKAAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQAVRTSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - DNA, 2208 bp, unidentified
  source 1..2208: mol_type = other DNA; note = Dependoparvovirus adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 14 - DNA, 2211 bp, unidentified
  source 1..2211: mol_type = other DNA; note = Dependoparvovirus adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcgcttcaacgggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacgccaagtctgccaatgtggactttactgtggacaataatggcgtgtattcagagcctcgccccattggcaccagatacctgactcgtaatctgtaa

SEQ ID NO: 15 - DNA, 2211 bp, unidentified
  source 1..2211: mol_type = other DNA; note = Dependoparvovirus adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcgcttcaacgggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaaggtatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacgccaagtctgccaatgtggactttactgtggacaataatggcgtgtattcagagcctcgccccattggcaccagatacctgactcgtaatctgtaa

SEQ ID NO: 16 - DNA, 2211 bp, unidentified
  source 1..2211: mol_type = other DNA; note = Dependoparvovirus adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcgcttcaacgggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaacttcgccaagtctgccaatgtggactttactgtggacaataatggcgtgtattcagagcctcgccccattggcaccagattcctgactcgtaatctgtaa

SEQ ID NO: 17 - DNA, 2211 bp, unidentified
  source 1..2211: mol_type = other DNA; note = Dependoparvovirus adeno-associated virus
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagcgcttcaacgggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaaggtatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaacttcgccaagtctgccaatgtggactttactgtggacaataatggcgtgtattcagagcctcgccccattggcaccagattcctgactcgtaatctgtaa

SEQ ID NO: 18 - DNA, 2175 bp, unidentified
  source 1..2175: mol_type = other DNA; note = Dependoparvovirus adeno-associated virus
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 19 - DNA, 2211 bp, unidentified
  source 1..2211: mol_type = other DNA; note = Dependoparvovirus adeno-associated virus
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 20 - DNA, 2211 bp, unidentified
  source 1..2211: mol_type = other DNA; note = Dependoparvovirus adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 21 - DNA, 2211 bp, unidentified
  source 1..2211: mol_type = other DNA; note = Dependoparvovirus adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccgttgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 22 - DNA, 2211 bp, unidentified
  source 1..2211: mol_type = other DNA; note = Dependoparvovirus adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaacttctacaagtctaataatgttgaatttgctgttaatactgaaggtgtatatagtgaaccccgccccattggcaccagatttctgactcgtaatctgtaa

SEQ ID NO: 23 - DNA, 2211 bp, unidentified
  source 1..2211: mol_type = other DNA; note = Dependoparvovirus adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccgttgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaacttctacaagtctaataatgttgaatttgctgttaatactgaaggtgtatatagtgaaccccgccccattggcaccagatttctgactcgtaatctgtaa

SEQ ID NO: 24 - DNA, 2232 bp, unidentified
  source 1..2232: mol_type = other DNA; note = Dependoparvovirus adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaaggcggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaacaggcggttaggacgtctttggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 25 - protein, 502 aa, Homo sapiens
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRTLYYGLNLLIPCVLISALALLVFLLPADSGEKISLGITVLLSLTVFMLLVAEIMPATSDSVPLIAQYFASTMIIVGLSVVVTVIVLQYHHHDPDGGKMPKWTRVILLNWCAWFLRMKRPGEDKVRPACQHKQRRCSLASVEMSAVAPPPASNGNLLYIGFRGLDGVHCVPTPDSGVVCGRMACSPTHDEHLLHGGQPPEGDPDLAKILEEVRYIANRFRCQDESEAVCSEWKFAACVVDRLCLMAFSVFTIICTIGILMSAPNFVEAVSKDFA

SEQ ID NO: 26 - protein, 457 aa, Homo sapiens
MYSFNTLRLYLWETIVFFSLAASKEAEAARSAPKPMSPSDFLDKLMGRTSGYDARIRPNFKGPPVNVSCNIFINSFGSIAETTMDYRVNIFLRQQWNDPRLAYNEYPDDSLDLDPSMLDSIWKPDLFFANEKGAHFHEITTDNKLLRISRNGNVLYSIRITLTLACPMDLKNFPMDVQTCIMQLESFGYTMNDLIFEWQEQGAVQVADGLTLPQFILKEEKDLRYCTKHYNTGKFTCIEARFHLERQMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 27 - protein, 452 aa, Homo sapiens [sequence omitted]

SEQ ID NO: 28 - protein, 464 aa, Homo sapiens
MAHVRHFRTLVSGFYFWEAALLLSLVATKETDSARSRSAPMSPSDFLDKLMGRTSGYDARIRPNFKGPPVNVTCNIFINSFGSIAETTMDYRVNIFLRQKWNDPRLAYSEYPDDSLDLDPSMLDSIWKPDLFFANEKGANFHEVTTDNKLLRIFKNGNVLYSIRLTLTLSCPMDLKNFPMDVQTCIMQLESFGYTMNDLIFEWQDEAPVQVAEGLTLPQFLLKEEKDLRYCTKHYNTGKFTCIEVRFHLERQMGYYLIQMYIPSLLIVILSWVSFWINMDAAPARVALGITTVLTMTTQSSGSRASLPKVSYVKAIDIWMAVCLLFVFSALLEYAAVNFVSRQHKELLRFRRKRKNKTEAFALEKFYRFSDMDDEVRESRFSFTAYGMGPCLQAKDGMTPKGPNHPVQVMPKSPDEMRKVFIDRAKKIDTISRACFPLAFLIFNIFYWVIYKILRHEDIHQQQD

SEQ ID NO: 29 - protein, 449 aa, Homo sapiens
MAHVRHFRTLVSGFYFWEAALLLSLVATKETDSARSRSAPMSPSDFLDKLMGRTSGYDARIRPNFKGPPVNVTCNIFINSFGSIAETTMDYRVNIFLRQKWNDPRLAYSEYPDDSLDLDPSMLDSIWKPDLFFANEKGANFHEVTTDNKLLRIFKNGNVLYSIRLTLTLSCPMDLKNFPMDVQTCIMQLESFGYTMNDLIFEWQDEAPVQVAEGLTLPQFLLKEEKDLRYCTKHYNTGKFTCIEVRFHLERQMGYYLIQMYIPSLLIVILSWVSFWINMDAAPARVALGITTVLTMTTQSSGSRASLPKVSYVKAIDIWMAVCLLFVFSALLEYAAVNFVSRQHKELLRFRRKRKNKDDEVRESRFSFTAYGMGPCLQAKDGMTPKGPNHPVQVMPKSPDEMRKVFIDRAKKIDTISRACFPLAFLIFNIFYWVIYKILRHEDIHQQQD

SEQ ID NO: 30 - protein, 439 aa, synthetic construct
MRCSPGGVWLALAASLLHVSLQGEFQRKLYKELVKNYNPLERPVANDSQPLTVYFSLSLLQIMDVDETTMVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 31 - protein, 439 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCYIDVRWFPFDVQHCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 32 - DNA, 1320 bp, synthetic construct
  source 1..1320: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtga

SEQ ID NO: 33 - protein, 439 aa, synthetic construct
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVLVNSSGHCQYLPPGIFKSSCPMDLKNFPMDVQTCKLKFGSWSYGGWSLDLQMQEADISGYIPNGEWDLVGIPGKRSERFYECCKEPYPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKSPMLNLFQEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 34 - protein, 22 aa, synthetic construct
EKISLGITVLLSLTVFMLLVAE

SEQ ID NO: 35 - protein, 23 aa, synthetic construct
PAKIGLGITVLLSLTTFMSGVAN

SEQ ID NO: 36 - DNA, 1359 bp, synthetic construct
  source 1..1359: mol_type = other DNA
atgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtga

SEQ ID NO: 37 - DNA, 305 bp, synthetic construct
  source 1..305: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 38 - DNA, 303 bp, synthetic construct
  source 1..303: mol_type = other DNA
ctagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaat

SEQ ID NO: 39 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 40 - DNA, 84 bp, synthetic construct
  source 1..84: mol_type = other DNA
tgcaaagccatttcagcaccacggagagtgcctctgcagctgcaaagccatttcagcaccacggagagtgcctctgctttttct

SEQ ID NO: 41 - DNA, 280 bp, synthetic construct
  source 1..280: mol_type = other DNA
tggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 42 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 43 - DNA, 1045 bp, synthetic construct
  source 1..1045: mol_type = other DNA
gaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 44 - DNA, 281 bp, synthetic construct
  source 1..281: mol_type = other DNA
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 45 - DNA, 1099 bp, synthetic construct
  source 1..1099: mol_type = other DNA
ctgcaggggaaagttggtggtgtatgcagctggacctaggagagaagcaggagaggaagatccagcacaaaaaatctgaagctaaaaacaggacacagagatgggggaagaaaagagggcagagtgaggcaaaaagagactgaagagatgagggtggccgccaggcactttagataggggagaggctttatttacctctgtttgtttttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctggattacaggggtgagccacagcgcctggtccctatttacttctgtcttctacctccaggagatcaaagacgctggccttcagacctgatcagactcccaggggcagccaccacatgtatgacagagaacagaggatgcctgtttttccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtccccc

SEQ ID NO: 46 - DNA, 1074 bp, synthetic construct
  source 1..1074: mol_type = other DNA
accgtgcatccagagcctggaagagtgcctggcacatagcaggtagtcaataaatgaatgggggcaagcagccaaatcagaatcaggttttcttgctaagcatagaactaacagaaggatcattgaatggattggataatgactggcatcagggtaaggtccccttaacaaacactcctgtcctgaacacctggttagctaacagttttctcatactcttattttcccaaaacacaattgctggatctcagctccaaatcaactcttctaggaaagtgaaaaattgctggatctcagctcccaaatcatatcttccaggcagagctaacattgccccttattcacacctccaccaaaccatctgatccaacagtgacaggtgtcacgaggccttggcatgcactctcttcccccgccagagttctgcgaaagccagggttgcgatttgttgtcagtttattccccgcctctatgagagtgtgagcactgggcaggctcggatgaaataatgcattgagtaggcctctgaaaccaaggcccctcagctggggcaacgtcaggctccagggtgggcaactttgctgcttctgccgaagatagtgatattgagaaaatgtgggtgcaatgaaacgcttattgcagcgcactcggtgcatctgtggacagagggtcaatcgcccctcagagcagcgcaaacaggcgtcccaagcctaggccttcacttgccccagcatccgcaagggtccattaatccttaacattcaaattccgcccactaaaccagtccctccgcgcccaagccgcctcttttccccaaacgacggccgaaagcagccaatcaacagctggaggggtccgcccccttttccctggccgaaatggacaggaaatctcgccaattgacggcatcgccgctgagactcccccctcccccgtcctccccgtcccagcccggccatcacagccaatgacgggcgggctcgcagcggcgccgagggcggggcgcgggcgcgcaggtgcagcagcgcgcgggccggccaagagggcggggcgcgacgtcggccgtgcggggtc

SEQ ID NO: 47 - DNA, 1482 bp, synthetic construct
  source 1..1482: mol_type = other DNA
acgcgtcgatccacagtctcctgagtagctgggactacaggagcttgttaccacacccagctccagtttataaattcatctccagtttataaaggaggaaaccgaggtactgagaggttaaaaaaccttcctgcagacacttgtccagcaagtggccactccaggatttggaccaaggtgatgtgtcttcaggctgtgtctctgccactgtgccacgctgctgggtggtaggcagcagtgggtgggtgcctgcagtggtctgtaaagaccacctgagatgtccttcctcctctgttccaccctgtccaggtccaagaagacagtctatgaagagagagcaggtgtgactctctcagtgtgctcctctgtgagaagcaggctgacatcccaaagggaagggcggataacagagacagtgcaagcggaggagatgagggtgcctcaaagccgggaggctgggtgatgcaggagcctgcgtgtcccgaggggggtgctgggcccagtgtgagtacgtgtgactgtgactgagacagtgtgactgctgaaggcagggacacagcagctccctgactgggggcagaaggcgttaactgtgtgaaggctggttgtgggtgggtgggctctgggcctcgaacccgggggctgagggagatagtaaacagcagggtgactgacgggaagatcatgttggtagccctgcgaagatgctgcagggctgtgggggtttgtgtgactttgcagttcaacaaattcaaattcagccaacgctggcagggcctgttgtgccaggcaaccagctaggaggaggagactcggacccagcttgcagctgaagggcgctggctgccgggttctgtgggttcaccttgcggtgtcttcccttgctaacactgagtccttacaatagccccatctccaggttgaggctagatggaggggacagagggaagtgacttgcccaaggtgacccaagctcccgagtgccagggcaggatctgaattcaggctctcagactgcagagcctgagtccctccctgccatgcctgtgccagggtggaaatgtctggtcctggaggggagcgtggactcctggccttggctctggagacatccccctagaccacgtgggctcctaacctgtccatggtcactgtgctgaggggcgggacggtgggtcacccctagttcttttttccccagggccagattcatggactgaagggttgctcggctctcagagtccccctaagcgccccgccctggccccaagccctcccccagctcccgcgtcccccccctcctggcgctgactccgggccagaagaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctccctaggggatcc

SEQ ID NO: 48 - DNA, 422 bp, synthetic construct
  source 1..422: mol_type = other DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 49 - DNA, 485 bp, synthetic construct
  source 1..485: mol_type = other DNA
ctagacagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagag

SEQ ID NO: 50 - DNA, 173 bp, synthetic construct
  source 1..173: mol_type = other DNA
agctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctg

SEQ ID NO: 51 - DNA, 296 bp, synthetic construct
  source 1..296: mol_type = other DNA
acttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtca

SEQ ID NO: 52 - DNA, 57 bp, synthetic construct
  source 1..57: mol_type = other DNA
agcttgcttgttctttttgcagaagctcagaataaacgctcaactttggccgccacc

SEQ ID NO: 53 - DNA, 83 bp, synthetic construct
  source 1..83: mol_type = other DNA
tcagaagccccgggctcgtcagtcaaaccggttctctgtttgcactcggcagcacgggcaggcaagtggtccctaggttcggg

SEQ ID NO: 54 - DNA, 126 bp, synthetic construct
  source 1..126: mol_type = other DNA
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 55 - DNA, 106 bp, synthetic construct
  source 1..106: mol_type = other DNA
gtcaagccggttctccgtttgcactcaggagcacgggcaggcgagtggcccctagttctgggggcagctctagagcggtaccggatcctctagagtcgactccgga

SEQ ID NO: 56 - DNA, 106 bp, synthetic construct
  source 1..106: mol_type = other DNA
gtcaagccggttctccgtttgcactcaggagcacgggcaggtgagtggcccctagttctgggggcagctctagagcggtaccggatcctctagagtcgactccgga

SEQ ID NO: 57 - DNA, 1068 bp, synthetic construct
  source 1..1068: mol_type = other DNA
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 58 - DNA, 235 bp, synthetic construct
  source 1..235: mol_type = other DNA
gtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggagcctctgctaaccatgttcatgccttcttctttttcctacagctcctgggcaacgtgctggttattgtgctgtctcatcattttggcaaagaattc

SEQ ID NO: 59 - DNA, 104 bp, synthetic construct
  source 1..104: mol_type = other DNA
gtaagttgggacatttggacctgggggtcagttgtccatactgcaaactgagtggccatttttgtgtgatgtaggtgaatcaccagtttttttctccttcccag

SEQ ID NO: 60 - DNA, 128 bp, synthetic construct
  source 1..128: mol_type = other DNA
gtgagtggctttggttcccggctgaggtggagtgggctgaggactagactgagccctcggacatggaggtggggatggggcagactcatcccattcttgaccaagcccttgttctgctcccttcccag

SEQ ID NO: 61 - DNA, 498 bp, synthetic construct
  source 1..498: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagactgcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacag

SEQ ID NO: 62 - DNA, 300 bp, synthetic construct
  source 1..300: mol_type = other DNA
ggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 63 - DNA, 810 bp, synthetic construct
  source 1..810: mol_type = other DNA
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 64 - DNA, 755 bp, synthetic construct
  source 1..755: mol_type = other DNA
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 65 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 66 - DNA, 94 bp, synthetic construct
  source 1..94: mol_type = other DNA
gctggagcctcggtagccgttcctcctgcccgctgggcctcccaacgggccctcctcccctccttgcaccggcccttcctggtctttgaataaa

SEQ ID NO: 67 - DNA, 228 bp, synthetic construct
  source 1..228: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 68 - DNA, 202 bp, synthetic construct
  source 1..202: mol_type = other DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 69 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 70 - DNA, 449 bp, synthetic construct
  source 1..449: mol_type = other DNA
gatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 71 - DNA, 3333 bp, synthetic construct
  source 1..3333: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgctggaggtgagtggctttggttcccggctgaggtggagtgggctgaggactagactgagccctcggacatggaggtggggatggggcagactcatcccattcttgaccaagcccttgttctgctcccttcccaggctctgtaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 72 - DNA, 3691 bp, synthetic construct
  source 1..3691: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatcctctagagtcgactccggactcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagactgcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccagcgtctcaaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatg

SEQ ID NO: 73 - DNA, 3652 bp, synthetic construct
  source 1..3652: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcatggaggtgagtggctttggttcccggctgaggtggagtgggctgaggactagactgagccctcggacatggaggtggggatggggcagactcatcccattcttgaccaagcccttgttctgctcccttcccaggctctgtaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 74 - DNA, 4099 bp, synthetic construct
  source 1..4099: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaaccgtgcatccagagcctggaagagtgcctggcacatagcaggtagtcaataaatgaatgggggcaagcagccaaatcagaatcaggttttcttgctaagcatagaactaacagaaggatcattgaatggattggataatgactggcatcagggtaaggtccccttaacaaacactcctgtcctgaacacctggttagctaacagttttctcatactcttattttcccaaaacacaattgctggatctcagctccaaatcaactcttctaggaaagtgaaaaattgctggatctcagctcccaaatcatatcttccaggcagagctaacattgccccttattcacacctccaccaaaccatctgatccaacagtgacaggtgtcacgaggccttggcatgcactctcttcccccgccagagttctgcgaaagccagggttgcgatttgttgtcagtttattccccgcctctatgagagtgtgagcactgggcaggctcggatgaaataatgcattgagtaggcctctgaaaccaaggcccctcagctggggcaacgtcaggctccagggtgggcaactttgctgcttctgccgaagatagtgatattgagaaaatgtgggtgcaatgaaacgcttattgcagcgcactcggtgcatctgtggacagagggtcaatcgcccctcagagcagcgcaaacaggcgtcccaagcctaggccttcacttgccccagcatccgcaagggtccattaatccttaacattcaaattccgcccactaaaccagtccctccgcgcccaagccgcctcttttccccaaacgacggccgaaagcagccaatcaacagctggaggggtccgcccccttttccctggccgaaatggacaggaaatctcgccaattgacggcatcgccgctgagactcccccctcccccgtcctccccgtcccagcccggccatcacagccaatgacgggcgggctcgcagcggcgccgagggcggggcgcgggcgcgcaggtgcagcagcgcgcgggccggccaagagggcggggcgcgacgtcggccgtgcggggtcccggcgtcggcggcgcgcgcgctccctcctctcggagagagggctgtggtaactcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagactgcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccagcgtctcaaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 75 - DNA, 4101 bp, synthetic construct
  source 1..4101: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtggtccctaggttcgggctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagactgcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccagcgtctcaaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 76 - DNA, 4110 bp, synthetic construct
  source 1..4110: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcaggggaaagttggtggtgtatgcagctggacctaggagagaagcaggagaggaagatccagcacaaaaaatctgaagctaaaaacaggacacagagatgggggaagaaaagagggcagagtgaggcaaaaagagactgaagagatgagggtggccgccaggcactttagataggggagaggctttatttacctctgtttgtttttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctggattacaggggtgagccacagcgcctggtccctatttacttctgtcttctacctccaggagatcaaagacgctggccttcagacctgatcagactcccaggggcagccaccacatgtatgacagagaacagaggatgcctgtttttccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtcccccacccgcgctcgtacgtgcgcctccgccggcagctcctgctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagactgcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccagcgtctcaaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 77 - DNA, 3928 bp, synthetic construct
  source 1..3928: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcaaagccatttcagcaccacggagagtgcctctgcagctgcaaagccatttcagcaccacggagagtgcctctgctttttctccatggtggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcttgcttgttctttttgcagaagctcagaataaacgctcaactttggccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 78 - DNA, 4047 bp, synthetic construct
  source 1..4047: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaacgcgtcgatccacagtctcctgagtagctgggactacaggagcttgttaccacacccagctccagtttataaattcatctccagtttataaaggaggaaaccgaggtactgagaggttaaaaaaccttcctgcagacacttgtccagcaagtggccactccaggatttggaccaaggtgatgtgtcttcaggctgtgtctctgccactgtgccacgctgctgggtggtaggcagcagtgggtgggtgcctgcagtggtctgtaaagaccacctgagatgtccttcctcctctgttccaccctgtccaggtccaagaagacagtctatgaagagagagcaggtgtgactctctcagtgtgctcctctgtgagaagcaggctgacatcccaaagggaagggcggataacagagacagtgcaagcggaggagatgagggtgcctcaaagccgggaggctgggtgatgcaggagcctgcgtgtcccgaggggggtgctgggcccagtgtgagtacgtgtgactgtgactgagacagtgtgactgctgaaggcagggacacagcagctccctgactgggggcagaaggcgttaactgtgtgaaggctggttgtgggtgggtgggctctgggcctcgaacccgggggctgagggagatagtaaacagcagggtgactgacgggaagatcatgttggtagccctgcgaagatgctgcagggctgtgggggtttgtgtgactttgcagttcaacaaattcaaattcagccaacgctggcagggcctgttgtgccaggcaaccagctaggaggaggagactcggacccagcttgcagctgaagggcgctggctgccgggttctgtgggttcaccttgcggtgtcttcccttgctaacactgagtccttacaatagccccatctccaggttgaggctagatggaggggacagagggaagtgacttgcccaaggtgacccaagctcccgagtgccagggcaggatctgaattcaggctctcagactgcagagcctgagtccctccctgccatgcctgtgccagggtggaaatgtctggtcctggaggggagcgtggactcctggccttggctctggagacatccccctagaccacgtgggctcctaacctgtccatggtcactgtgctgaggggcgggacggtgggtcacccctagttcttttttccccagggccagattcatggactgaagggttgctcggctctcagagtccccctaagcgccccgccctggccccaagccctcccccagctcccgcgtcccccccctcctggcgctgactccgggccagaagaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctccctaggggatcccaggtaagttgggacatttggacctgggggtcagttgtccatactgcaaactgagtggccatttttgtgtgatgtaggtgaatcaccagtttttttctccttcccaggtgtaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 79 - DNA, 3106 bp, synthetic construct
  source 1..3106: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgccaggtaagttgggacatttggacctgggggtcagttgtccatactgcaaactgagtggccatttttgtgtgatgtaggtgaatcaccagtttttttctccttcccaggtgtaaaacaaatcaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 80 - DNA, 3417 bp, synthetic construct
  source 1..3417: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggtgagtggcccctagttctgggggcagctctagagcggtaccggatcctctagagtcgactccggacctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggagcctctgctaaccatgttcatgccttcttctttttcctacagctcctgggcaacgtgctggttattgtgctgtctcatcattttggcaaagaattcaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 81 - DNA, 3425 bp, synthetic construct
  source 1..3425: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcacaggtaagttgggacatttggacctgggggtcagttgtccatactgcaaactgagtggccatttttgtgtgatgtaggtgaatcaccagtttttttctccttcccaggtgtaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 82 - DNA, 4142 bp, synthetic construct
  source 1..4142: mol_type = other DNA
tccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaaccgtgcatccagagcctggaagagtgcctggcacatagcaggtagtcaataaatgaatgggggcaagcagccaaatcagaatcaggttttcttgctaagcatagaactaacagaaggatcattgaatggattggataatgactggcatcagggtaaggtccccttaacaaacactcctgtcctgaacacctggttagctaacagttttctcatactcttattttcccaaaacacaattgctggatctcagctccaaatcaactcttctaggaaagtgaaaaattgctggatctcagctcccaaatcatatcttccaggcagagctaacattgccccttattcacacctccaccaaaccatctgatccaacagtgacaggtgtcacgaggccttggcatgcactctcttcccccgccagagttctgcgaaagccagggttgcgatttgttgtcagtttattccccgcctctatgagagtgtgagcactgggcaggctcggatgaaataatgcattgagtaggcctctgaaaccaaggcccctcagctggggcaacgtcaggctccagggtgggcaactttgctgcttctgccgaagatagtgatattgagaaaatgtgggtgcaatgaaacgcttattgcagcgcactcggtgcatctgtggacagagggtcaatcgcccctcagagcagcgcaaacaggcgtcccaagcctaggccttcacttgccccagcatccgcaagggtccattaatccttaacattcaaattccgcccactaaaccagtccctccgcgcccaagccgcctcttttccccaaacgacggccgaaagcagccaatcaacagctggaggggtccgcccccttttccctggccgaaatggacaggaaatctcgccaattgacggcatcgccgctgagactcccccctcccccgtcctccccgtcccagcccggccatcacagccaatgacgggcgggctcgcagcggcgccgagggcggggcgcgggcgcgcaggtgcagcagcgcgcgggccggccaagagggcggggcgcgacgtcggccgtgcggggtcccggcgtcggcggcgcgcgcgctccctcctctcggagagagggctgtggtaacctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggagcctctgctaaccatgttcatgccttcttctttttcctacagctcctgggcaacgtgctggttattgtgctgtctcatcattttggcaaagaattcaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 83 - DNA, 4143 bp, synthetic construct
  source 1..4143: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtggtccctaggttcgggcctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggagcctctgctaaccatgttcatgccttcttctttttcctacagctcctgggcaacgtgctggttattgtgctgtctcatcattttggcaaagaattcaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 84 - DNA, 4152 bp, synthetic construct
  source 1..4152: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcaggggaaagttggtggtgtatgcagctggacctaggagagaagcaggagaggaagatccagcacaaaaaatctgaagctaaaaacaggacacagagatgggggaagaaaagagggcagagtgaggcaaaaagagactgaagagatgagggtggccgccaggcactttagataggggagaggctttatttacctctgtttgtttttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctggattacaggggtgagccacagcgcctggtccctatttacttctgtcttctacctccaggagatcaaagacgctggccttcagacctgatcagactcccaggggcagccaccacatgtatgacagagaacagaggatgcctgtttttccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtcccccacccgcgctcgtacgtgcgcctccgccggcagctcctgcctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggagcctctgctaaccatgttcatgccttcttctttttcctacagctcctgggcaacgtgctggttattgtgctgtctcatcattttggcaaagaattcaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 85 - DNA, 4240 bp, synthetic construct
  source 1..4240: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcaaagccatttcagcaccacggagagtgcctctgcagctgcaaagccatttcagcaccacggagagtgcctctgctttttctccatggtggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcttgcttgttctttttgcagaagctcagaataaacgctcaactttggccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 86 - DNA, 4248 bp, synthetic construct
  source 1..4248: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaacgcgtcgatccacagtctcctgagtagctgggactacaggagcttgttaccacacccagctccagtttataaattcatctccagtttataaaggaggaaaccgaggtactgagaggttaaaaaaccttcctgcagacacttgtccagcaagtggccactccaggatttggaccaaggtgatgtgtcttcaggctgtgtctctgccactgtgccacgctgctgggtggtaggcagcagtgggtgggtgcctgcagtggtctgtaaagaccacctgagatgtccttcctcctctgttccaccctgtccaggtccaagaagacagtctatgaagagagagcaggtgtgactctctcagtgtgctcctctgtgagaagcaggctgacatcccaaagggaagggcggataacagagacagtgcaagcggaggagatgagggtgcctcaaagccgggaggctgggtgatgcaggagcctgcgtgtcccgaggggggtgctgggcccagtgtgagtacgtgtgactgtgactgagacagtgtgactgctgaaggcagggacacagcagctccctgactgggggcagaaggcgttaactgtgtgaaggctggttgtgggtgggtgggctctgggcctcgaacccgggggctgagggagatagtaaacagcagggtgactgacgggaagatcatgttggtagccctgcgaagatgctgcagggctgtgggggtttgtgtgactttgcagttcaacaaattcaaattcagccaacgctggcagggcctgttgtgccaggcaaccagctaggaggaggagactcggacccagcttgcagctgaagggcgctggctgccgggttctgtgggttcaccttgcggtgtcttcccttgctaacactgagtccttacaatagccccatctccaggttgaggctagatggaggggacagagggaagtgacttgcccaaggtgacccaagctcccgagtgccagggcaggatctgaattcaggctctcagactgcagagcctgagtccctccctgccatgcctgtgccagggtggaaatgtctggtcctggaggggagcgtggactcctggccttggctctggagacatccccctagaccacgtgggctcctaacctgtccatggtcactgtgctgaggggcgggacggtgggtcacccctagttcttttttccccagggccagattcatggactgaagggttgctcggctctcagagtccccctaagcgccccgccctggccccaagccctcccccagctcccgcgtcccccccctcctggcgctgactccgggccagaagaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctccctaggggatccaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 87 - DNA, 3838 bp, synthetic construct
  source 1..3838: mol_type = other DNA
ccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcttgcttgttctttttgcagaagctcagaataaacgctcaactttggccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 88 - DNA, 3295 bp, synthetic construct
  source 1..3295: mol_type = other DNA
ctagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaactagacagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 89 - DNA, 2988 bp, synthetic construct
  source 1..2988: mol_type = other DNA
ctagacagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 90 - DNA, 3040 bp, synthetic construct
  source 1..3040: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgcaaagccatttcagcaccacggagagtgcctctgcagctgcaaagccatttcagcaccacggagagtgcctctgctttttcttgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 91 - DNA, 2956 bp, synthetic construct
  source 1..2956: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 92 - DNA, 3858 bp, synthetic construct
  source 1..3858: mol_type = other DNA
ctagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 93 - DNA, 3094 bp, synthetic construct
  source 1..3094: mol_type = other DNA
ctagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaaacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 94 - DNA, 168 bp, synthetic construct
  source 1..168: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 95 - DNA, 168 bp, synthetic construct
  source 1..168: mol_type = other DNA
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 96 - DNA, 4582 bp, synthetic construct
  source 1..4582: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgctggaggtgagtggctttggttcccggctgaggtggagtgggctgaggactagactgagccctcggacatggaggtggggatggggcagactcatcccattcttgaccaagcccttgttctgctcccttcccaggctctgtaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgtaacccaggctggagtgcagtggcacacactgctcactgaaacctcctcctccctgggttcaagcacttctcctgcctaggcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatgtgtggggccttgtagtctcagctacttgggagggtgaggcaggaggatttcttgaactgggaggcagagcttgcagtgagcccactgcactccagcctgggcaacagagtagaactcctctcgaatgaaagaaaaaaaagtggactgtgaaaactgaaaggactagaaaaactcaagtcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 97 - DNA, 4567 bp, synthetic construct
  source 1..4567: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagctctagagcggtaccggatcctctagagtcgactccggactcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagactgcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccagcgtctcaaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgtaacccaggctggagtgcagtggcacacactgctcactgaaacctcctcctccctgggttcaagcacttctcctgcctaggcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgatcaagtcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 98 - DNA, 4601 bp, synthetic construct
  source 1..4601: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcatggaggtgagtggctttggttcccggctgaggtggagtgggctgaggactagactgagccctcggacatggaggtggggatggggcagactcatcccattcttgaccaagcccttgttctgctcccttcccaggctctgtaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgtaacccaggctggagtgcagtggcacacactgctcactgaaacctcctcctccctgggttcaagcacttctcctgcctaggcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctcaagtcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 99 - DNA, 4448 bp, synthetic construct
  source 1..4448: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaaccgtgcatccagagcctggaagagtgcctggcacatagcaggtagtcaataaatgaatgggggcaagcagccaaatcagaatcaggttttcttgctaagcatagaactaacagaaggatcattgaatggattggataatgactggcatcagggtaaggtccccttaacaaacactcctgtcctgaacacctggttagctaacagttttctcatactcttattttcccaaaacacaattgctggatctcagctccaaatcaactcttctaggaaagtgaaaaattgctggatctcagctcccaaatcatatcttccaggcagagctaacattgccccttattcacacctccaccaaaccatctgatccaacagtgacaggtgtcacgaggccttggcatgcactctcttcccccgccagagttctgcgaaagccagggttgcgatttgttgtcagtttattccccgcctctatgagagtgtgagcactgggcaggctcggatgaaataatgcattgagtaggcctctgaaaccaaggcccctcagctggggcaacgtcaggctccagggtgggcaactttgctgcttctgccgaagatagtgatattgagaaaatgtgggtgcaatgaaacgcttattgcagcgcactcggtgcatctgtggacagagggtcaatcgcccctcagagcagcgcaaacaggcgtcccaagcctaggccttcacttgccccagcatccgcaagggtccattaatccttaacattcaaattccgcccactaaaccagtccctccgcgcccaagccgcctcttttccccaaacgacggccgaaagcagccaatcaacagctggaggggtccgcccccttttccctggccgaaatggacaggaaatctcgccaattgacggcatcgccgctgagactcccccctcccccgtcctccccgtcccagcccggccatcacagccaatgacgggcgggctcgcagcggcgccgagggcggggcgcgggcgcgcaggtgcagcagcgcgcgggccggccaagagggcggggcgcgacgtcggccgtgcggggtcccggcgtcggcggcgcgcgcgctccctcctctcggagagagggctgtggtaactcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagactgcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccagcgtctcaaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatctcaagtcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 100 - DNA, 4450 bp, synthetic construct
  source 1..4450: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtggtccctaggttcgggctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagactgcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccagcgtctcaaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatctcaagtcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 101 - DNA, 4459 bp, synthetic construct
  source 1..4459: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcaggggaaagttggtggtgtatgcagctggacctaggagagaagcaggagaggaagatccagcacaaaaaatctgaagctaaaaacaggacacagagatgggggaagaaaagagggcagagtgaggcaaaaagagactgaagagatgagggtggccgccaggcactttagataggggagaggctttatttacctctgtttgtttttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctggattacaggggtgagccacagcgcctggtccctatttacttctgtcttctacctccaggagatcaaagacgctggccttcagacctgatcagactcccaggggcagccaccacatgtatgacagagaacagaggatgcctgtttttccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtcccccacccgcgctcgtacgtgcgcctccgccggcagctcctgctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagactgcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccagcgtctcaaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatctcaagtcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 102 - DNA, 4593 bp, synthetic construct
  source 1..4593: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcaaagccatttcagcaccacggagagtgcctctgcagctgcaaagccatttcagcaccacggagagtgcctctgctttttctccatggtggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcttgcttgttctttttgcagaagctcagaataaacgctcaactttggccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgtaacccaggctggagtgcagtggcacacactgctcactgaaacctcctcctccctgggttcaagcacttctcctgcctaggcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcaagtcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 103 - DNA, 4593 bp, synthetic construct
  source 1..4593: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaacgcgtcgatccacagtctcctgagtagctgggactacaggagcttgttaccacacccagctccagtttataaattcatctccagtttataaaggaggaaaccgaggtactgagaggttaaaaaaccttcctgcagacacttgtccagcaagtggccactccaggatttggaccaaggtgatgtgtcttcaggctgtgtctctgccactgtgccacgctgctgggtggtaggcagcagtgggtgggtgcctgcagtggtctgtaaagaccacctgagatgtccttcctcctctgttccaccctgtccaggtccaagaagacagtctatgaagagagagcaggtgtgactctctcagtgtgctcctctgtgagaagcaggctgacatcccaaagggaagggcggataacagagacagtgcaagcggaggagatgagggtgcctcaaagccgggaggctgggtgatgcaggagcctgcgtgtcccgaggggggtgctgggcccagtgtgagtacgtgtgactgtgactgagacagtgtgactgctgaaggcagggacacagcagctccctgactgggggcagaaggcgttaactgtgtgaaggctggttgtgggtgggtgggctctgggcctcgaacccgggggctgagggagatagtaaacagcagggtgactgacgggaagatcatgttggtagccctgcgaagatgctgcagggctgtgggggtttgtgtgactttgcagttcaacaaattcaaattcagccaacgctggcagggcctgttgtgccaggcaaccagctaggaggaggagactcggacccagcttgcagctgaagggcgctggctgccgggttctgtgggttcaccttgcggtgtcttcccttgctaacactgagtccttacaatagccccatctccaggttgaggctagatggaggggacagagggaagtgacttgcccaaggtgacccaagctcccgagtgccagggcaggatctgaattcaggctctcagactgcagagcctgagtccctccctgccatgcctgtgccagggtggaaatgtctggtcctggaggggagcgtggactcctggccttggctctggagacatccccctagaccacgtgggctcctaacctgtccatggtcactgtgctgaggggcgggacggtgggtcacccctagttcttttttccccagggccagattcatggactgaagggttgctcggctctcagagtccccctaagcgccccgccctggccccaagccctcccccagctcccgcgtcccccccctcctggcgctgactccgggccagaagaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctccctaggggatcccaggtaagttgggacatttggacctgggggtcagttgtccatactgcaaactgagtggccatttttgtgtgatgtaggtgaatcaccagtttttttctccttcccaggtgtaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgtaacccaggctggagtgcagtggcacacactgctcactgaaacctcctcctccctgggttcaagcacttctcctgcctaggcctcccaagtagctgtgattataggtgcacaccaccaagcctggctaatttttatatctttagtagagacgggagtctcaccatgttggccaggctagtctctggcaagccatggtcaagtcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 104 - DNA, 4550 bp, synthetic construct
  source 1..4550: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgccaggtaagttgggacatttggacctgggggtcagttgtccatactgcaaactgagtggccatttttgtgtgatgtaggtgaatcaccagtttttttctccttcccaggtgtaaaacaaatcaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtaacccaggctggagtgcagtggcacacactgctcactgaaacctcctcctccctgggttcaagcacttctcctgcctaggcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatgtgtggggccttgtagtctcagctacttgggagggtgaggcaggaggatttcttgaactgggaggcagagcttgcagtgagcccactgcactccagcctgggcaacagagtagaactcctctcgaatgaaagaaaaaaaagtggactgtgaaaactgaaaggactagaaaaactacactacaaagctacagaaaccaagaaagcaccatagcgtttgccttcaactgcttcccaacttgttttcctcttccaatttgattgtggtttcctctccagaaggaactccacagtacttagcgttggccagataataggttctcaaatacttgttaataaataagtttgttcgagaagctgggcagtgatattctactcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 105 - DNA, 4561 bp, synthetic construct
  source 1..4561: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggtgagtggcccctagttctgggggcagctctagagcggtaccggatcctctagagtcgactccggacctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggagcctctgctaaccatgttcatgccttcttctttttcctacagctcctgggcaacgtgctggttattgtgctgtctcatcattttggcaaagaattcaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtaacccaggctggagtgcagtggcacacactgctcactgaaacctcctcctccctgggttcaagcacttctcctgcctaggcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatgtgtggggccttgtagtctcagctacttgggagggtgaggcaggaggatttcttgaactgggaggcagagctttcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 106 - DNA, 4569 bp, synthetic construct
  source 1..4569: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcacaggtaagttgggacatttggacctgggggtcagttgtccatactgcaaactgagtggccatttttgtgtgatgtaggtgaatcaccagtttttttctccttcccaggtgtaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtaacccaggctggagtgcagtggcacacactgctcactgaaacctcctcctccctgggttcaagcacttctcctgcctaggcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatgtgtggggccttgtagtctcagctacttgggagggtgaggcaggaggatttcttgaactgggaggcagagctttcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 107 - DNA, 4485 bp, synthetic construct
  source 1..4485: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaaccgtgcatccagagcctggaagagtgcctggcacatagcaggtagtcaataaatgaatgggggcaagcagccaaatcagaatcaggttttcttgctaagcatagaactaacagaaggatcattgaatggattggataatgactggcatcagggtaaggtccccttaacaaacactcctgtcctgaacacctggttagctaacagttttctcatactcttattttcccaaaacacaattgctggatctcagctccaaatcaactcttctaggaaagtgaaaaattgctggatctcagctcccaaatcatatcttccaggcagagctaacattgccccttattcacacctccaccaaaccatctgatccaacagtgacaggtgtcacgaggccttggcatgcactctcttcccccgccagagttctgcgaaagccagggttgcgatttgttgtcagtttattccccgcctctatgagagtgtgagcactgggcaggctcggatgaaataatgcattgagtaggcctctgaaaccaaggcccctcagctggggcaacgtcaggctccagggtgggcaactttgctgcttctgccgaagatagtgatattgagaaaatgtgggtgcaatgaaacgcttattgcagcgcactcggtgcatctgtggacagagggtcaatcgcccctcagagcagcgcaaacaggcgtcccaagcctaggccttcacttgccccagcatccgcaagggtccattaatccttaacattcaaattccgcccactaaaccagtccctccgcgcccaagccgcctcttttccccaaacgacggccgaaagcagccaatcaacagctggaggggtccgcccccttttccctggccgaaatggacaggaaatctcgccaattgacggcatcgccgctgagactcccccctcccccgtcctccccgtcccagcccggccatcacagccaatgacgggcgggctcgcagcggcgccgagggcggggcgcgggcgcgcaggtgcagcagcgcgcgggccggccaagagggcggggcgcgacgtcggccgtgcggggtcccggcgtcggcggcgcgcgcgctccctcctctcggagagagggctgtggtaacctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggagcctctgctaaccatgttcatgccttcttctttttcctacagctcctgggcaacgtgctggttattgtgctgtctcatcattttggcaaagaattcaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 108 - DNA, 4487 bp, synthetic construct
  source 1..4487: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcagaagccccgggctcgtcagtcaaaccggttctctgtttgcactcggcagcacgggcaggcaagtggtccctaggttcgggcctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggagcctctgctaaccatgttcatgccttcttctttttcctacagctcctgggcaacgtgctggttattgtgctgtctcatcattttggcaaagaattcaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 109 - DNA, 4496 bp, synthetic construct
  source 1..4496: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccactgcaggggaaagttggtggtgtatgcagctggacctaggagagaagcaggagaggaagatccagcacaaaaaatctgaagctaaaaacaggacacagagatgggggaagaaaagagggcagagtgaggcaaaaagagactgaagagatgagggtggccgccaggcactttagataggggagaggctttatttacctctgtttgtttttttttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctggattacaggggtgagccacagcgcctggtccctatttacttctgtcttctacctccaggagatcaaagacgctggccttcagacctgatcagactcccaggggcagccaccacatgtatgacagagaacagaggatgcctgtttttccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtcccccacccgcgctcgtacgtgcgcctccgccggcagctcctgcctggcagaactcggtaagtctgttgacatgtatgtgatgtatactaacctgcatgggacgtggatttacttgtgtatgtcagatagagtaaagattaactcttgcatgtgagcggggcatcgagatagcgataaatgagtcaggaggacggagcctctgctaaccatgttcatgccttcttctttttcctacagctcctgggcaacgtgctggttattgtgctgtctcatcattttggcaaagaattcaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 110 - DNA, 4600 bp, synthetic construct
  source 1..4600: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcaaagccatttcagcaccacggagagtgcctctgcagctgcaaagccatttcagcaccacggagagtgcctctgctttttctccatggtggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcttgcttgttctttttgcagaagctcagaataaacgctcaactttggccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 111 - DNA, 4592 bp, synthetic construct
  source 1..4592: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccaacgcgtcgatccacagtctcctgagtagctgggactacaggagcttgttaccacacccagctccagtttataaattcatctccagtttataaaggaggaaaccgaggtactgagaggttaaaaaaccttcctgcagacacttgtccagcaagtggccactccaggatttggaccaaggtgatgtgtcttcaggctgtgtctctgccactgtgccacgctgctgggtggtaggcagcagtgggtgggtgcctgcagtggtctgtaaagaccacctgagatgtccttcctcctctgttccaccctgtccaggtccaagaagacagtctatgaagagagagcaggtgtgactctctcagtgtgctcctctgtgagaagcaggctgacatcccaaagggaagggcggataacagagacagtgcaagcggaggagatgagggtgcctcaaagccgggaggctgggtgatgcaggagcctgcgtgtcccgaggggggtgctgggcccagtgtgagtacgtgtgactgtgactgagacagtgtgactgctgaaggcagggacacagcagctccctgactgggggcagaaggcgttaactgtgtgaaggctggttgtgggtgggtgggctctgggcctcgaacccgggggctgagggagatagtaaacagcagggtgactgacgggaagatcatgttggtagccctgcgaagatgctgcagggctgtgggggtttgtgtgactttgcagttcaacaaattcaaattcagccaacgctggcagggcctgttgtgccaggcaaccagctaggaggaggagactcggacccagcttgcagctgaagggcgctggctgccgggttctgtgggttcaccttgcggtgtcttcccttgctaacactgagtccttacaatagccccatctccaggttgaggctagatggaggggacagagggaagtgacttgcccaaggtgacccaagctcccgagtgccagggcaggatctgaattcaggctctcagactgcagagcctgagtccctccctgccatgcctgtgccagggtggaaatgtctggtcctggaggggagcgtggactcctggccttggctctggagacatccccctagaccacgtgggctcctaacctgtccatggtcactgtgctgaggggcgggacggtgggtcacccctagttcttttttccccagggccagattcatggactgaagggttgctcggctctcagagtccccctaagcgccccgccctggccccaagccctcccccagctcccgcgtcccccccctcctggcgctgactccgggccagaagaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctccctaggggatccaaaacaaaacaaaagccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattattcctgttaatcaacctctggattacaaaatttgtgaaagattgactggtattcttaactatgttgctccttttacgctatgtggatacgctgctttaatgcctttgtatcatgctattgcttcccgtatggctttcattttctcctcctagtataaatcctggttgctgtctctttatgaggagttgtggcccgtagtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggactagctggagcctcggtagccgttcctcctgcccgctgggcctcccaacgggccctcctcccctccttgcaccggcccttcctggtctttgaataaattcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 112 - DNA, 4503 bp, synthetic construct
  source 1..4503: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagcttgcttgttctttttgcagaagctcagaataaacgctcaactttggccgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgatggataattaattaattaggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttagatctttttccctctgccaaaaattatggggacatcatgaagccccttgagcatctgacttctggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgtaacccaggctggagtgcagtggcacacactgctcactgaaacctcctcctccctgggttcaagcacttctcctgcctaggcctcccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcaagtcagtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 113 - DNA, 3623 bp, synthetic construct
  source 1..3623: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttactagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaactagacagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 114 - DNA, 3330 bp, synthetic construct
  source 1..3330: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttactagtttaattaaactagacagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 115 - DNA, 3356 bp, synthetic construct
  source 1..3356: mol_type = other DNA
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgcaaagccatttcagcaccacggagagtgcctctgcagctgcaaagccatttcagcaccacggagagtgcctctgctttttcttgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 116 - DNA, 3272 bp, synthetic construct
  source 1..3272: mol_type = other DNA
ctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccaggtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 117 - DNA, 4186 bp, synthetic construct
  source 1..4186: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttactagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 118 - DNA, 3422 bp, synthetic construct
  source 1..3422: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttactagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaaacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagggaggctccggtggggactacaaagacgatgacgacaagtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 119 - DNA, 119 bp, synthetic construct
  source 1..119: mol_type = other DNA
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 120 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 121 - DNA, 3286 bp, synthetic construct
  source 1..3286: mol_type = other DNA
gcggccgcacgcgtgttactagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaactagacagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtgaagcggccgcgacgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 122 - DNA, 3849 bp, synthetic construct
  source 1..3849: mol_type = other DNA
gcggccgcacgcgtgttactagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtgaagcggccgcgacgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 123 - DNA, 2983 bp, synthetic construct
  source 1..2983: mol_type = other DNA
gcggccgcacgcgtgttataaactagacagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtgaagcggccgcgacgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 124 - DNA, 3546 bp, synthetic construct
  source 1..3546: mol_type = other DNA
gcggccgcacgcgtgttataaagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtgaagcggccgcgacgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 125 - DNA, 4553 bp, synthetic construct
  source 1..4553: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttactagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaactagacagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtgaagcggccgcgacgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtagcaattgccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatgtgtggggccttgtagtctcagctacttgggagggtgaggcaggaggatttcttgaactgggaggcagagcttgcagtgagcccactgcactccagcctgggcaacagagtagaactcctctcgaatgaaagaaaaaaaagtggactgtgaaaactgaaaggactagaaaaactacactacaaagctacagaaaccaagaaagcaccatagcgtttgccttcaactgcttcccaacttgttttcctcttccaatttgattgtggtttcctctccagaaggaactccacagtacttagcgttggccagatgctagccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 126 - DNA, 4566 bp, synthetic construct
  source 1..4566: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttactagtacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccattaattaaagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtgaagcggccgcgacgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtagcaattgccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattgctagccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 127 - DNA, 4551 bp, synthetic construct
  source 1..4551: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttataaactagacagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtgaagcggccgcgacgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtagcaattgccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattatgttgaaaacatagggtctggggaaaaagggatttaaaataagaagaaaaagaagacttgggacttaaaaagtcttttagaggccagctcaccaacattcagaacacccagtctgtgttgcacaatatgttacttaggtataaatcaaggattcatgtaattttgtcattccttgcgtgatattttaaaaaacattctgtgtaaggtatttataaagctctcttctaaaaatacaaaaatgtgtggggccttgtagtctcagctacttgggagggtgaggcaggaggatttcttgaactgggaggcagagcttgcagtgagcccactgcactccagcctgggcaacagagtagaactcctctcgaatgaaagaaaaaaaagtggactgtgaaaactgaaaggactagaaaaactacactacaaagctacagaaaccaagaaagcaccatagcgtttgccttcaactgcttcccaacttgttttcctcttccaatttgattgtggtttcctctccagaaggaactccacagtacttagcgttggccagatggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattgctagccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 128 - DNA, 4549 bp, synthetic construct
  source 1..4549: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttataaagaagttggcagggaaggtgcccagaaaattttggattgaagatttcatggcaagtctctggccagtggcctagcccgggtaagccatgctatgctcacctccccacagccccctctcgcctttttttttttttttttttttaccttgactggaagcacaagcagaaactgggacatgagcaccaggagtccagatttccatggtcccgttgggggcatggggttggggagaggttgcagaggagggctctggaggggagcaactgtcacagctgtgagaggtgggggtgagcaggcagtcagggctgttccctccagaatcctggggtgtcctctgcacttctgcgccaagctggagtgctagtgtgatggacaaggtggtaagagagctgaaagagcacgagcataacaagaaagacagaggcagaagcaaaaaaaaaaaaaaaaaaaaaacagagggcaacagagagacagttacagagactacagtgatccacagagggagagccatccctgtgaattagccatcatttccctgtaaaccttagaacccagctgttgccagggcaacggggcaatacctgtctctctagagatgaagttgccagggtaactgcatcctgtcattcgttcctggggaccatccggaatgcggcacccactggctgttaccatggcaactgcctttttgccccacttaatcccatcccgtctgctacaagggccccacagttggaggtgggggaggtgggaagagaaaagatcacttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgctcggatccccgggtaccggtcgccaccatgcgctgctcgccgggaggcgtctggctggcgctggccgcgtcgctcctgcacgtgtccctgcaaggcgagttccagaggaagctttacaaggagctggtcaagaactacaatcccttggagaggcccgtggccaatgactcgcaaccactcaccgtctacttctccctgagcctcctgcagatcatggacgtggatgagaagaaccaagttttaaccaccaacatttggctgcaaatgtcttggacagatcactatttacagtggaatgtgtcagaatatccaggggtgaagactgttcgtttcccagatggccagatttggaaaccagacattcttctctataacagtgctgatgagcgctttgacgccacattccacactaacgtgttggtgaactcttctgggcattgccagtacctgcctccaggcatattcaagagttcctgccccatggacttgaagaatttccccatggatgtccagacatgcaaactgaagtttgggtcctggtcttacggaggctggtccttggatctgcagatgcaggaggcagatatcagtggctatatccccaatggagaatgggacctagtgggaatccccggcaagaggagtgaaaggttctatgagtgctgcaaagagccctaccccgatgtcaccttcacagtgaccatgcgccgcaggatgggttactacctgattcagatgtatattcccagcctgctcattgtcatcctctcatggatctccttctggatcaacatggatgctgcacctgctcgtgtgggcctaggcatcaccactgtgctcaccatgaccacccagagctccggctctcgagcatctctgcccaaggtgtcctatgtgaaagccattgacatttggatggcagtttgcctgctctttgtgttctcagccctattagaatatgctgccgttaactttgtgtctcggcaacataaggagctgctccgattcaggaggaagcggagacatcacaagagccccatgttgaatctattccaggaggatgaagctggagaaggccgctttaacttctctgcctatgggatgggcccagcctgtctacaggccaaggatggcatctcagtcaagggcgccaacaacagtaacaccaccaacccccctcctgcaccatctaagtccccagaggagatgcgaaaactcttcatccagagggccaagaagatcgacaaaatatcccgcattggcttccccatggccttcctcattttcaacatgttctactggatcatctacaagattgtccgtagagaggacgtccacaaccagtgaagcggccgcgacgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatctctagaagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtagcaattgccaagtagctgtgattataggtgcacaccaccaagcctggctaatttttatatctttagtagagacgggagtctcaccatgttggccaggctagtctctggcaagccatggtaaaatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattgatgtaactatttaaggctgctttaaattagactaatagcagagtggtcagactatactgaaagcttggtgaatcacaattaagtacctcaaagaactattcttgtttgccttattcctatgtaaataactgaaatctttgtttttcttcctaaaaggggtcatgttgatttttacttacaatgtattttaagtttgtcactctaaatggttatgagcaagtttaagaaaaatcttcagcaaatactaccttagattatgaccccaaaacacatttacttatgattgctagccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

(19) United States
(12) Patent Application Publication
(10) Pub. No.: US 2025/0090689 A1
(43) Pub. Date: Mar. 20, 2025

(54) EXPRESSION CASSETTES FOR TREATING EPILEPSY AND NEUROPATHIC PAIN

(71) Applicant: Trames Bio, Inc., Oakland, CA (US)

(72) Inventor: Annahita Keravala, Palo Alto, CA (US)

(21) Appl. No.: 18/729,329

(22) PCT Filed: Jan. 31, 2023
(86) PCT No.: PCT/US2023/061702

§ 371 Date: Jul. 16, 2024

Related U.S. Application Data
(60) Provisional application No. 63/304,960, filed on Jan. 31, 2022.
(60) Provisional application No. 63/312,480, filed on Feb. 22, 2022.

(51) Int. Cl. A61K 48/00; C12N 15/86

(52) U.S. Cl. CPC A61K 48/0058; C12N 15/86; C12N 2750/14122; C12N 2750/14143; C12N 2750/14145; C12N 2750/14152

(57) ABSTRACT

The present disclosure provides expression cassettes, vectors and methods for expressing a transgene in a cell, e.g., a neuron. Further provided are methods of achieving desirable expression level of a transgene in a neuron cell, e.g., for use in combination with a small molecule ligand to treat focal epilepsy or neuropathic pain.

### CROSS-REFERENCE TO RELATED APPLICATIONS

This application claims the benefit of U.S. Provisional Application No. 63/304,960, filed Jan. 31, 2022 and U.S. Provisional Application No. 63/312,480, filed Feb. 22, 2022, the content of each of which is herein incorporated by reference in its entirety.

### REFERENCE TO AN ELECTRONIC SEQUENCE LISTING

The content of the electronic sequence listing (SWCH_038_02WO_SeqList_ST26.xml; Size: 383,720 bytes; and Date of Creation: Jan. 29, 2023) is herein incorporated by reference in its entirety.

### BACKGROUND

Intractable neurological disease is often associated with aberrantly acting neurons. Attempts to develop therapies to treat these conditions have been hampered by a lack of tractable target proteins associated with the disease.

Focal epilepsy is a chronic and debilitating neurological disorder that affects over 2 million people in the U.S. and is characterized by unpredictable seizures initiated from a specific location in the brain. Recurrent seizures result in cognitive and emotional deficits, with current interventions offering limited efficacy and multiple side effects. Focal seizures originate from aberrant firing in a subset of neurons, yet most anti-epileptic drugs rely on systemic compound administration and alter brain-wide activity. Thus, patients with epilepsy endure substantial side effects due to unintentional modulation of neurons involved in normal cognition and undesirable off-target changes in other biological systems. Mesial temporal lobe epilepsy (mTLE) is the most common form of focal epilepsy, and it is commonly associated with hippocampal sclerosis as the histopathological abnormality.

Neuropathic pain includes Peripheral Neuropathy and Trigeminal Neuralgia, among others. The former is a common neurological disorder resulting from the damage to, or dysfunction of, the peripheral nervous system. It is characterized by numbness, tingling and pain, often starting in the hands and feet but can also affect other areas of your body. Trigeminal Neuralgia, also known as Suicide Disease, is caused by damage to the myelin sheath protecting the trigeminal cranial nerve. It brings about extreme and sporadic shock-like facial pain that can last from a few seconds to a couple minutes. Current pharmacological and surgical approaches provide little relief and a potential for addiction while having significant side effects.

The most commonly used therapy for chronic pain is the application of opioid analgesics and nonsteroidal anti-inflammatory drugs, but these drugs can lead to addiction and may cause side effects, such as drug dependence, tolerance, respiratory depression, sedation, cognitive failure, hallucinations, and other systemic side effects. Despite the wide usage of pharmaceuticals, there is a strikingly low success rate for its effectiveness in pain relief. More invasive options for the treatment of pain include nerve blocks and electrical stimulation. The most invasive, and least preferred, method for managing pain is complete surgical removal of the nerve or section thereof that is causing the pain.

Ideal pain and focal epilepsy treatments would only alter activity of the specific neurons responsible for the pain and/or seizure generation. Thus, a new strategy for treating pain or focal epilepsy is to deliver heterologous proteins encoded by recombinant nucleic acid into a sub-population of neurons to control the aberrant activity.

One consideration of this strategy is the choice of heterologous proteins for controlling neuronal activity and its expression in the target cells. Recently, Designer Receptors Exclusively Activated by Designer Drugs (DREADDs) to alter seizure activity have produced promising results in animal epilepsy models. However, inhibitory DREADDs rely on G protein-coupled receptors indirectly activating ion channels through second messengers and can inconsistently change neuron electrical potentials.

An alternative treatment approach is to transduce the target neurons with a transgene encoding an engineered ligand-gated ion channel (LGIC) that can subsequently respond to an exogenous ligand. Oral administration of this small molecule ligand, which is designed to only interact with the engineered LGIC, can be finely tuned to control the aberrant activity of neurons and suppress seizures without adverse effects.

There is a need in the art for designing optimal expression cassettes that allow robust expression of the transgene in the target cells for controlling neuronal activity. Such expression cassettes can be used for treating neuropathic pain, such as Peripheral Neuropathy and Trigeminal Neuralgia, or focal epilepsy, such as mesial temporal lobe epilepsy. The present disclosure provides such expression cassettes, vectors, methods, and more.

### SUMMARY

In one aspect, the present disclosure provides recombinant nucleic acids comprising an expression cassette comprising, in 5′ to 3′ order, one or more of a 5′ enhancer, a promoter, a 5′ untranslated region (UTR), a transgene, a 3′ enhancer, and a polyadenylation sequence (polyA), wherein the transgene is operably linked to the promoter.

In some embodiments, the expression cassette comprises the 5′ enhancer comprising a polynucleotide sequence at least 90% identical to any one of SEQ ID NO: 37-39.

In some embodiments, the expression cassette does not comprise the 5′ enhancer.

In some embodiments, the promoter comprising a polynucleotide sequence at least 90% identical to any one of SEQ ID NO: 41-51. In some embodiments, the promoter is a neuron-specific promoter.

In some embodiments, the expression cassette comprises an intron between the promoter and the transgene. In some embodiments, the expression cassette comprises an intron between the 5′ UTR and the transgene. In some embodiments, the expression cassette comprises an intron between the promoter and the 5′ UTR. In some embodiments, the intron comprises a polynucleotide sequence at least 90% identical to any one of SEQ ID NO: 57-61.

In some embodiments, the expression cassette does not comprise an intron.

In some embodiments, the expression cassette comprises the 5′ UTR comprising a polynucleotide sequence at least 90% identical to any one of SEQ ID NO: 52-56.

In some embodiments, the expression cassette comprises the 3′ enhancer comprising a polynucleotide sequence at least 90% identical to any one of SEQ ID NO: 62-65.

In some embodiments, the expression cassette comprises the polyA comprising a polynucleotide sequence at least 90% identical to any one of SEQ ID NO: 67-70.

In some embodiments, the expression cassette comprises a non-neuron silencing element embedded in the promoter, and wherein the non-neuron silencing element comprises a polynucleotide sequence at least 90% identical to SEQ ID NO: 40. In some embodiments, the expression cassette comprises a non-neuron silencing element between the 5′ enhancer and the promoter, and wherein the non-neuron silencing element comprises a polynucleotide sequence at least 90% identical to SEQ ID NO: 40.

In some embodiments, the expression cassette comprises a 3′ UTR between the 3′ enhancer and the polyA, and wherein the 3′ UTR comprises a polynucleotide sequence at least 90% identical to SEQ ID NO: 66.

In some embodiments, the transgene encodes a ligand-gated ion channel (LGIC). In some embodiments, the ligand-gated ion channel comprises a ligand binding domain derived from human a7 nicotinic acetylcholine receptor (a7-nAChR). In some embodiments, the ligand binding domain comprises an amino acid sequence having at least 85% identity to amino acid residues 23-220 of SEQ ID NO: 25. In some embodiments, the ligand binding domain comprises one or more amino acid mutations listed in Table 5. In some embodiments, ligand-gated ion channel comprises an ion pore domain derived from a human Glycine receptor. In some embodiments, the ion pore domain comprises an amino acid sequence having at least 85% identity to amino acids 255-457 of SEQ ID NO: 26, 260-452 of SEQ ID NO: 27, amino acids 259-464 of SEQ ID NO: 28, or amino acids 259-449 of SEQ ID NO: 29. In some embodiments, the ligand binding domain of the engineered receptor comprises a Cys-loop domain derived from the human Glycine receptor. In some embodiments, the ligand-gated ion channel comprises an amino acid sequence having at least 95% sequence identity to any one of SEQ ID NO: 25-31 and 33. In some embodiments, the human Glycine receptor is human Glycine receptor α1, and wherein the ligand-gated ion channel comprises an amino acid sequence having at least 95% sequence identity to SEQ ID NO: 33. In some embodiments, the transgene comprises or consists of a polynucleotide sequence having at least 90%, at least 95%, at least 96%, at least 97%, at least 98%, at least 99%, at least 99.5%, or 100% identity to SEQ ID NO: 32. In some embodiments, the transgene is codon-optimized for expression in a human cell. In some embodiments, the human cell is a neuron.

In some embodiments, the expression cassette comprises, in 5′ to 3′ order: (i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37; (ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43; (iii) a hCaMKII 5′ UTR comprising a polynucleotide sequence having at least 90%, at least 95%, at least 96%, at least 97%, at least 98%, at least 99%, at least 99.5%, or 100% identity to SEQ ID NO: 53; (iv) a hCMV+rGlob Intron comprising a polynucleotide sequence having at least 90%, at least 95%, at least 96%, at least 97%, at least 98%, at least 99%, at least 99.5%, or 100% identity to SEQ ID NO: 58; (v) the transgene; (vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64; (vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and (viii) a hGH polyA comprising a polynucleotide sequence having at least 90%, at least 95%, at least 96%, at least 97%, at least 98%, at least 99%, at least 99.5%, or 100% identity to SEQ ID NO: 68.

In some embodiments, the expression cassette comprises, in 5′ to 3′ order, a sequence having at least 90%, at least 95%, at least 96%, at least 97%, at least 98%, at least 99%, at least 99.5%, or 100% identity to nucleotides 173-1877 of SEQ ID NO: 108, the transgene, and a sequence having at least 90%, at least 95%, at least 96%, at least 97%, at least 98%, at least 99%, at least 99.5%, or 100% identity to nucleotides 3237-4315 of SEQ ID NO: 108.

In some embodiments, the expression cassette comprises, in 5′ to 3′ order: (i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37; (ii) a hSyn promoter comprising a polynucleotide sequence having at least 90%, at least 95%, at least 96%, at least 97%, at least 98%, at least 99%, at least 99.5%, or 100% identity to SEQ ID NO: 48; (iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54; (iv) a hSyn intron comprising a polynucleotide sequence having at least 90%, at least 95%, at least 96%, at least 97%, at least 98%, at least 99%, at least 99.5%, or 100% identity to SEQ ID NO: 59; (v) the transgene; (vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64; (vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and (viii) a hGH polyA comprising a polynucleotide sequence having at least 90%, at least 95%, at least 96%, at least 97%, at least 98%, at least 99%, at least 99.5%, or 100% identity to SEQ ID NO: 68.

In some embodiments, the expression cassette comprises, in 5′ to 3′ order, a sequence having at least 90%, at least 95%, at least 96%, at least 97%, at least 98%, at least 99%, at least 99.5%, or 100% identity to nucleotides 173-1159 of SEQ ID NO: 106, the transgene, and a sequence having at least 90%, at least 95%, at least 96%, at least 97%, at least 98%, at least 99%, at least 99.5%, or 100% identity to nucleotides 2519-3597 of SEQ ID NO: 106.

In some embodiments, the expression cassette comprises, in 5′ to 3′ order: (i) a CMV-V2 5′ enhancer comprising a polynucleotide sequence having at least 90%, at least 95%, at least 96%, at least 97%, at least 98%, at least 99%, at least 99.5%, or 100% identity to SEQ ID NO: 38; (ii) a hSyn-V2 promoter comprising a polynucleotide sequence having at least 90%, at least 95%, at least 96%, at least 97%, at least 98%, at least 99%, at least 99.5%, or 100% identity to SEQ ID NO: 49; (iii) the transgene; (iv) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and (v) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

In some embodiments, the expression cassette comprises, in 5′ to 3′ order, a sequence having at least 90%, at least 95%, at least 96%, at least 97%, at least 98%, at least 99%, at least 99.5%, or 100% identity to nucleotides 149-946 of SEQ ID NO: 125, the transgene, and a sequence having at least 90%, at least 95%, at least 96%, at least 97%, at least 98%, at least 99%, at least 99.5%, or 100% identity to nucleotides 2285-3416 of SEQ ID NO: 125.

In some embodiments, the expression cassette comprises, in 5′ to 3′ order: (i) a CMV-V2 5′ enhancer comprising a polynucleotide sequence having at least 90%, at least 95%, at least 96%, at least 97%, at least 98%, at least 99%, at least 99.5%, or 100% identity to SEQ ID NO: 38; (ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43; (iii) the transgene; (iv) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and (v) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

In some embodiments, the expression cassette comprises, in 5′ to 3′ order, a sequence having at least 90%, at least 95%, at least 96%, at least 97%, at least 98%, at least 99%, at least 99.5%, or 100% identity to nucleotides 149-1527 of SEQ ID NO: 126, the transgene, and a sequence having at least 90%, at least 95%, at least 96%, at least 97%, at least 98%, at least 99%, at least 99.5%, or 100% identity to nucleotides 2848-3979 of SEQ ID NO: 126.

In some embodiments, the expression cassette comprises, in 5′ to 3′ order: (i) a hSyn-V2 promoter comprising a polynucleotide sequence having at least 90%, at least 95%, at least 96%, at least 97%, at least 98%, at least 99%, at least 99.5%, or 100% identity to SEQ ID NO: 49; (ii) the transgene; (iii) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and (iv) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

In some embodiments, the expression cassette comprises, in 5′ to 3′ order, a sequence having at least 90%, at least 95%, at least 96%, at least 97%, at least 98%, at least 99%, at least 99.5%, or 100% identity to nucleotides 153-661 of SEQ ID NO: 127, the transgene, and a sequence having at least 90%, at least 95%, at least 96%, at least 97%, at least 98%, at least 99%, at least 99.5%, or 100% identity to nucleotides 1982-3113 of SEQ ID NO: 127.

In some embodiments, the expression cassette comprises, in 5′ to 3′ order: (i) a hCaMKIIa promoter comprising a polynucleotide sequence having at least 90%, at least 95%, at least 96%, at least 97%, at least 98%, at least 99%, at least 99.5%, or 100% identity to SEQ ID NO: 43; (ii) the transgene; (iii) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and (iv) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

In some embodiments, the expression cassette comprises, in 5′ to 3′ order, a sequence having at least 90%, at least 95%, at least 96%, at least 97%, at least 98%, at least 99%, at least 99.5%, or 100% identity to nucleotides 153-1224 of SEQ ID NO: 128, the transgene, and a sequence having at least 90%, at least 95%, at least 96%, at least 97%, at least 98%, at least 99%, at least 99.5%, or 100% identity to nucleotides 2545-3676 of SEQ ID NO: 128.

In some embodiments, the expression cassette comprises, in 5′ to 3′ order: (i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37; (ii) a hSyn promoter comprising a polynucleotide sequence having at least 90%, at least 95%, at least 96%, at least 97%, at least 98%, at least 99%, at least 99.5%, or 100% identity to SEQ ID NO: 48; (iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54; (iv) a hTPI intron comprising a polynucleotide sequence having at least 90%, at least 95%, at least 96%, at least 97%, at least 98%, at least 99%, at least 99.5%, or 100% identity to SEQ ID NO: 60; (v) the transgene; (vi) a FullEES 3′ enhancer comprising a polynucleotide sequence having at least 90%, at least 95%, at least 96%, at least 97%, at least 98%, at least 99%, at least 99.5%, or 100% identity to SEQ ID NO: 63; and (vii) a rpGlobin polyA comprising a polynucleotide sequence having at least 90%, at least 95%, at least 96%, at least 97%, at least 98%, at least 99%, at least 99.5%, or 100% identity to SEQ ID NO: 70.

In some embodiments, the expression cassette comprises, in 5′ to 3′ order, a sequence having at least 90%, at least 95%, at least 96%, at least 97%, at least 98%, at least 99%, at least 99.5%, or 100% identity to nucleotides 173-1188 of SEQ ID NO: 98, the transgene, and a sequence having at least 90%, at least 95%, at least 96%, at least 97%, at least 98%, at least 99%, at least 99.5%, or 100% identity to nucleotides 2548-3824 of SEQ ID NO: 98.

In some embodiments, the expression cassette comprises a polynucleotide sequence having at least 90% identity to any one of SEQ ID NO: 71-93 excluding the sequence of the transgene (SEQ ID NO: 36). In some embodiments, the expression cassette comprises a polynucleotide sequence having at least 90% identity to any one of SEQ ID NO: 121-124 excluding the sequence of the transgene (SEQ ID NO: 32). In some embodiments, the expression cassette comprises a polynucleotide sequence having at least 90% identity to any one of SEQ ID NO: 71-93 and 121-124.

In some embodiments, the recombinant nucleic acid comprises an adeno-associated virus (AAV) inverted terminal repeat (ITR) flanking each end of the expression cassette. In some embodiments, the recombinant nucleic acid comprises a 5′ ITR sequence having at least 90% identity to SEQ ID NO: 94 or 119 and a 3′ ITR sequence having at least 90% identity to SEQ ID NO: 95 or 120.

In some embodiments, the recombinant nucleic acid comprises a polynucleotide sequence having at least 90% identity to any one of SEQ ID NO: 96-118 and 125-128. In some embodiments, the recombinant nucleic acid comprises a polynucleotide sequence having at least 90% identity to any one of SEQ ID NO: 96-118 excluding the sequence of the transgene (SEQ ID NO: 36). In some embodiments, the recombinant nucleic acid comprises a polynucleotide sequence having at least 90% identity to any one of SEQ ID NO: 125-128 excluding the sequence of the transgene (SEQ ID NO: 32).

In one aspect, the disclosure provides vectors comprising the recombinant nucleic acid of the disclosure. In some embodiments, the vector is a non-viral vector. In some embodiments, the vector is a viral vector. In some embodiments, the vector comprises or consists of an AAV vector genome.

In one aspect, the disclosure provides AAVs comprising the vector of the disclosure. In some embodiments, the AAV is AAV9 serotype. In some embodiments, the AAV is a self-complementary AAV or a single stranded AAV. In some embodiments, the AAV is a wild-type AAV or a modified AAV. In some embodiments, the AAV comprises a capsid protein having at least 95% identity to an AAV9 capsid protein (SEQ ID NO: 8) or AAV9-TV capsid protein (SEQ ID NO: 9).

In one aspect, the disclosure provides host cells comprising the nucleic acid of the disclosure or the vector of the disclosure.

In one aspect, the disclosure provides methods of producing the AAV of the disclosure.

In one aspect, the disclosure provides kits comprising the recombinant nucleic acid of the disclosure, the vector of the disclosure, or the AAV of the disclosure.

In one aspect, the disclosure provides methods of expressing a transgene in a cell, comprising delivering the recombinant nucleic acid of the disclosure or the vector of the disclosure to the cell.

In one aspect, the disclosure provides methods of transducing a cell, comprises contacting the cell with the AAV of the disclosure. In some embodiments, the cell is a neuron.

In some embodiments, the neuron is a hippocampal neuron. In some embodiments, the neuron is an excitatory neuron. In some embodiments, the neuron is a CAMK2 positive neuron. In some embodiments, the neuron is an inhibitory neuron. In some embodiments, the neuron is a GABAergic neuron. In some embodiments, the neuron is a dorsal root ganglion neuron or a trigeminal ganglion neuron. In some embodiments, the neuron comprises an isolectin B4 (IB4) positive nerve fiber. In some embodiments, the neuron comprises an NF200 positive nerve fiber. In some embodiments, the neuron comprises a CGRP positive nerve fiber. In some embodiments, the neuron comprises a C fiber. In some embodiments, the neuron comprises an Aδ fiber. In some embodiments, the cell is an ex vivo cell. In some embodiments, the cell is an in vivo cell of a subject, optionally wherein the subject is a human. In some embodiments, the cell comprising the expression cassette has a higher expression level of the transgene compared to a corresponding cell comprising a control expression cassette, optionally wherein said higher expression is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least ten-fold higher than the transgene expression level of the control expression cassette comprising the polynucleotide sequence of SEQ ID NO: 88 excluding the transgene sequence. In some embodiments, the cell comprising the expression cassette has a comparable or higher expression level of the transgene compared to a corresponding cell comprising a control expression cassette, optionally wherein said transgene expression level is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least ten-fold of the transgene expression level of the control expression cassette comprising the polynucleotide sequence of SEQ ID NO: 87 excluding the transgene sequence.

In one aspect, the disclosure provides methods of treating a disease or disorder in a subject in need thereof, comprises administering an effective amount of the recombinant nucleic acid of the disclosure, the vector of the disclosure, or the AAV of the disclosure to the subject, wherein the disease or disorder is epilepsy, schizophrenia, autism spectrum disorder, Alzheimer's disease, Rett syndrome, or fragile X syndrome. In some embodiments, the epilepsy is focal epilepsy. In some embodiments, the epilepsy is mesial temporal lobe epilepsy (mTLE). In some embodiments, the recombinant nucleic acid, the vector, or the AAV is administered by intracranial administration, intrathecal (spine) administration, intrathecal (Cisterna magna) administration, intracerebral administration, intraventricular administration, or direct injection into the epileptic focus in hippocampus. In some embodiments, the recombinant nucleic acid, the vector, or the AAV is administered by direct injection into the epileptic focus in hippocampus. In some embodiments, 1×10⁹-1×10¹⁴ copies of the recombinant nucleic acid or AAV vector genome are administered to the subject. In some embodiments, the AAV comprises a capsid protein of AAV9 (SEQ ID NO: 8). In some embodiments, the method decreases the duration, intensity, and/or frequency of the epilepsy by at least 10%.

In one aspect, the disclosure provides methods of treating a disease or disorder in a subject in need thereof, comprises administering an effective amount of the recombinant nucleic acid of the disclosure, the vector of the disclosure, or the AAV of the disclosure to the subject, wherein the disease or disorder is neuropathic pain, spasticity, spinal cord injury, or avulsion injury. In some embodiments, the disease or disorder is neuropathic pain. In some embodiments, the neuropathic pain is peripheral neuropathy. In some embodiments, the neuropathic pain is trigeminal neuralgia. In some embodiments, the recombinant nucleic acid, the vector, or the AAV is administered by intrathecal (IT) or intraganglionic (IG) administration. In some embodiments, wherein the recombinant nucleic acid, the vector, or the AAV is administered by intraganglionic (IG) administration directly into dorsal root ganglion or trigeminal ganglion. In some embodiments, 1×10⁹-1×10¹⁴ copies of the recombinant nucleic acid or AAV vector genome are administered to the subject. In some embodiments, the AAV comprises a capsid protein of AAV9-TV (SEQ ID NO: 9). In some embodiments, the method lowers the level of the pain by at least 10%.

In some embodiments, the recombinant nucleic acid, the vector, or the AAV is administered by systemic, parenteral, intravenous, cerebral, cerebrospinal, intrathecal, intracisternal, intraputaminal, intrahippocampal, intra-striatal, or intra-cerebroventricular injection. In some embodiments, 1×10⁹-1×10¹⁴ copies of the recombinant nucleic acid or AAV vector genome are administered to the subject.

In some embodiments, the method comprises administering a ligand of the ligand-gated ion channel encoded by the transgene. In some embodiments, the ligand is selected from the group consisting of AZD-0328, TC-6987, ABT-126, TC-5619, TC-6683, Varenicline, and Facinicline/RG3487. In some embodiments, the ligand is TC-5619.

### BRIEF DESCRIPTION OF DRAWINGS

The patent or application file contains at least one drawing executed in color.

FIG. 1 shows the design of 27 expression cassettes. For each cassette, an element to the left is located upstream (5′) of an element to the right. The “*” sign in cassette #20 indicates that the NRSE element is embedded in, instead of being placed upstream of, the CMV promoter in this cassette.

FIG. 2 shows ddPCR results of CODA receptor mRNA ratio compared to the 3-actin housekeeping gene in SK-N-AS and HeLa cells transfected with the indicated cassettes.

FIG. 3 shows FLAG ELISA results of CODA receptor protein in SK-N-AS and HeLa cells transfected with the indicated cassettes.

FIG. 4 shows ddPCR results for CODA receptor mRNA expression ratio compared to the 3-actin housekeeping gene of nine expression cassettes packaged as AAV9 vectors and transduced in neonatal rat hippocampal mixed cultures.

FIG. 5 shows ddPCR results of CODA receptor mRNA ratio compared to the 3-actin housekeeping gene in rat hippocampi directly injected in vivo with AAV9-carrying six of the expression cassettes.

FIG. 6 shows localization of FLAG-tagged CODA receptor by immunofluorescence from the six expression cassettes tested in vivo in rat hippocampi.

FIG. 7 shows seizure frequency in KA focal epilepsy mouse model injected with AAVs comprising the indicated expression cassettes followed by repeat dosing of TC-5619.

FIG. 8 shows the design of efficacy study using KA focal epilepsy mouse model injected with AAVs comprising the indicated expression cassettes.

FIG. 9 shows ddPCR results for CODA receptor mRNA expression ratio compared to the j-actin housekeeping gene of ten expression cassettes packaged as AAV6 vectors and transduced in human iPSC derived sensory neuron progenitors.

FIG. 10 shows ddPCR results of CODA receptor mRNA ratio compared to the j-actin housekeeping gene in rat L3 and L4 DRGs directly injected in vivo with AAV6-carrying four of the expression cassettes.

FIG. 11 shows localization of FLAG-tagged CODA receptor by immunofluorescence from the four expression cassettes tested in vivo in rat L5 DRGs.

### DETAILED DESCRIPTION

The details of the disclosure are set forth in the accompanying description below. Although methods and materials similar or equivalent to those described herein can be used in the practice or testing of the present disclosure, illustrative methods, and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. Unless explicitly indicated otherwise, all specified some embodiments, features, and terms intend to include both the recited embodiment, feature, or term and biological equivalents thereof. The terminology used in the description herein is for the purpose of describing particular embodiments only and is not intended to be limiting.

Before the present methods and compositions are described, it is to be understood that this disclosure is not limited to particular method or composition described, as such may, of course, vary. It is also to be understood that the terminology used herein is for the purpose of describing particular embodiments only, and is not intended to be limiting, since the scope of the present disclosure will be limited only by the appended claims. Unless defined otherwise, all technical and scientific terms used herein have the same meaning as commonly understood by one of ordinary skill in the art to which this disclosure belongs.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

### Definitions

Throughout this disclosure, the singular forms “a,” “an” and “the” include plural references unless the content clearly dictates otherwise. Thus, for example, reference to “a cell” includes a plurality of such cells and reference to “the peptide” includes reference to one or more peptides and equivalents thereof, e.g., polypeptides, known to those skilled in the art, and so forth.

Throughout this specification, the term “and/or” is used in this disclosure to either “and” or “or” unless indicated otherwise.

Throughout this disclosure, unless the context requires otherwise, the word “comprise”, or variations such as “comprises” or “comprising” refers to the inclusion of a stated element or integer or group of elements or integers but not the exclusion of any other element or integer or group of elements or integers. Further, the statement of numerical ranges throughout this specification specifically includes all integers and decimal points in between.

Throughout this disclosure, unless the context requires otherwise, the phrase “consisting essentially of” refers to a limitation of the scope of composition, method, or kit described to the specified materials or steps that do not materially affect the basic and novel characteristic(s) of the subject disclosure. For example, a polypeptide “consisting essentially of” a disclosed sequence has the amino acid sequence of the disclosed sequence plus or minus about 5 amino acid residues at the boundaries of the sequence, e.g., about 5 residues, 4 residues, 3 residues, 2 residues or about 1 residue less than the recited bounding amino acid residue, or about 1 residue, 2 residues, 3 residues, 4 residues, or 5 residues more than the recited bounding amino acid residue.

Throughout this disclosure, unless the context requires otherwise, the phrase “consisting of” refers to the exclusion from the composition, method, or kit of any element, step, or ingredient not specified in the claim. For example, a polypeptide “consisting of” a disclosed sequence consists only of the disclosed amino acid sequence.

The terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 10% in either direction (greater than or less than) of the stated reference value unless otherwise stated or otherwise evident from the context (except where such number would exceed 100% of a possible value).

The term “isolated” means material that is substantially or essentially free from components that normally accompany is as found in its native state. In some embodiments, the term “obtained” is used synonymously with isolated.

The terms “subject,” “individual,” and “patient” are used interchangeably to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older.

The term “sample” refers to a volume and/or mass of biological material that is subjected to analysis. In some embodiments, a sample comprises a tissue sample, cell sample, a fluid sample, and the like. In some embodiments, a sample is taken from or provided by a subject (e.g., a human subject). In some embodiments, a sample comprises a portion of tissue taken from any internal organ, a cancerous, pre-cancerous, or non-cancerous tumor, brain, skin, hair (including roots), eye, muscle, bone marrow, cartilage, white adipose tissue, and/or brown adipose tissue. In some embodiments, a fluid sample comprises buccal swabs, blood, cord blood, saliva, semen, urine, ascites fluid, pleural fluid, spinal fluid, pulmonary lavage, tears, sweat, and the like. Those of ordinary skill in the art will appreciate that, in some embodiments, a “sample” is a “primary sample” in that it is obtained directly from a source (e.g., a subject). In some embodiments, a “sample” is the result of processing of a primary sample, for example to remove certain potentially contaminating components, to isolate certain components, and/or to purify certain components of interest. In some embodiments, a sample is a cell or population of cells (e.g., a neuronal cell). A cell sample may be derived directly from a subject (e.g., a primary sample) or may be a cell line. Cell lines may include non-mammalian cells (e.g., insect cells, yeast cells, and/or bacterial cells) or mammalian cells (e.g., immortalized cell lines).

The terms “treating”, “treatment” and the grammatical equivalents used herein generally refer to the use of a composition or method to reduce, eliminate, or prevent symptoms of a disease and includes achieving a therapeutic benefit and/or a prophylactic benefit. By therapeutic benefit is meant slowing the progression of, halting the progression of, reversing the progression of, or eradication or amelioration of the symptoms of the disorder or condition being treated. A prophylactic benefit of treatment includes reducing the risk of a condition, retarding the progress of a condition, or decreasing the likelihood of occurrence of a condition.

In some embodiments, treat refers to delivering a composition to a subject and/or population of cells to affect a physiologic outcome. In some embodiments, treatment results in an improvement (e.g., reduction, amelioration, or remediation) of one or more disease symptoms.

The improvement may be an observable or measurable improvement, or may be an improvement in the general feeling of well-being of the subject. Treatment of a disease can refer to a reduction in the severity of disease symptoms. In some embodiments, treatment can refer to a reduction in the severity of disease symptoms to levels comparable to those prior to disease onset. In some embodiments, treatment may refer to a short-term (e.g., temporary or acute) and/or a long-term (e.g., sustained or chronic) reduction in disease symptoms. In some embodiments, treatment may refer to a remission of disease symptoms. In some embodiments, treatment may refer to the prophylactic treatment of a subject at risk of developing a particular disease in order to prevent disease development. Prevention of disease development can refer to complete prevention of the disease symptoms, a delay in disease onset, a lessening of the severity of the symptoms in a subsequently developed disease, or reducing the likelihood of disease development.

The terms “management” or “controlling” refers to the use of the compositions or methods contemplated in the disclosure, to improve the quality of life for an individual suffering from a particular disease.

The term “effective amount” refers to an amount that can achieve the indicated result (e.g., transducing a target neuron). In some embodiments, an effective amount for the in vivo administration refers to a dose that can achieve a desirable result, such as, for example, transducing a sufficient amount of target neurons for therapeutic purpose.

A “therapeutically effective amount” is an amount of a composition that can achieve a desired therapeutic outcome. The therapeutically effective amount may vary according to factors such as, but not limited to, disease state and age, sex, and weight of the subject.

Generally, a therapeutically effective amount is also one in which any toxic or detrimental effects of a composition are outweighed by the therapeutically beneficial effects. A “therapeutically effective amount” includes an amount of a composition that is effective to treat a subject.

An “increase” refers to an increase in a value (e.g., increased transduction efficiency) of at least 5% as compared to a reference or control level. For example, an increase may include a5, 6, 7, 8, 9, 10, 15, 20, 25, 30, 35, 40, 45, 50, 60, 70, 80, 90, 100, 150, 200, 250, 500, 1000% or more increase. Increase also means an increase that is 1.1, 1.2, 1.5, 2, 3, 4, 5, 6, 7, 8, 9, 10, 15, 20, 30 or more times (e.g., 500, 1000 times) higher than a reference or control level.

A “decrease”, “reduce”, “diminish” or synonyms thereof refers to a decrease in a value (e.g., decreased transduction efficiency) of at least 5% as compared to a reference or control level. For example, a decrease may include a 5, 6, 7, 8, 9, 10, 15, 20, 25, 30, 35, 40, 45, 50, 60, 70, 80, 90, 100, 150, 200, 250, 500, 1000% or more decrease. Decrease also means a decrease that is 1.1, 1.2, 1.5, 2, 3, 4, 5, 6, 7, 8, 9, 10, 15, 20, 30 or more times (e.g., 500, 1000 times) lower than a reference or control level.

The terms “reference” or “control” level are used interchangeably throughout the disclosure and refer the value of a particular physiologic and/or therapeutic effect in a subject or sample that has not been treated with a composition of the disclosure, or a subject or sample that has been treated with a control. In some embodiments, the control is a vehicle control. In some embodiments, a reference level refers to a value of a particular physiologic and/or therapeutic effect that is measure in a subject or sample prior to the administration of a composition of the disclosure (e.g., a baseline level).

The term “ligand” refers to a molecule that binds to another, larger molecule. In some embodiments, the ligand binds to a receptor. In some embodiments, the binding of the ligand to the receptor alters the function of the receptor—to activate or repress its function. In some embodiments, the binding of the ligand to a receptor such as a ligand gated ion channel (LGIC) leads to the opening or closing of the ion channel. The term “ligand” may refer to an endogenous or naturally occurring ligand. For example, in some embodiments, a ligand refers to a neurotransmitter (e.g., λ-aminobutyric acid (GABA), acetylcholine, serotonin, and others) and signaling intermediate (e.g., phosphatidylinositol 4,5-bisphosphate (PIP2)), amino acids (e.g., glycine), or nucleotides (e.g., ATP). In some embodiments, a ligand may refer to a non-native, i.e., synthetic or non-naturally occurring, ligand (e.g., a binding agent). For example, in some embodiments, a ligand refers to a small molecule. Ligand binding can be measured by a variety of methods known in the art (e.g., detection of association with a radioactively labeled ligand). “Receptor-ligand binding” and “ligand binding” are used interchangeably throughout the disclosure and refer to the physical interaction between a receptor (e.g., a LGIC) and a ligand.

“Binding affinity” generally refers to the strength of the sum total of noncovalent interactions between a single binding site of a receptor and a ligand. Unless indicated otherwise, as used throughout the disclosure, “binding affinity” refers to intrinsic binding affinity which reflects a 1:1 interaction between members of a binding pair (e.g., receptor and ligand). The affinity of a molecule X for its partner Y can generally be represented by the dissociation constant (Kd). Affinity can be measured by common methods known in the art, including those described in the disclosure.

The term “wild type” or “native” is a term of the art understood by skilled persons and means the typical form of an organism, strain, gene, protein, or characteristic as it occurs in nature as distinguished from mutant or variant forms. For example, a wild type protein is the typical form of that protein as it occurs in nature.

The terms “non-native”, “variant”, and “mutant” are used interchangeably throughout the specification and the claims to refer to a mutant of a native, or wild type, composition, for example a variant polypeptide having less than 100% sequence identity with the native, or wild type, sequence.

The terms “polypeptide” and “protein” are used interchangeably herein to refer to polymers of amino acids. The terms also encompass a modified amino acid polymer; for example, disulfide bond formation, glycosylation, lipidation, phosphorylation, methylation, carboxylation, deamidation, acetylation, or conjugation with a labeling component.

Amino acid modifications may be amino acid substitutions, amino acid deletions and/or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g., charge, hydrophobicity and size). As used throughout the disclosure, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to praline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

The term “engineered” is used throughout the specification and claims to refer to a non-naturally occurring composition, or protein having properties that are distinct from the parental composition, or protein from which it was derivatized.

In general, “sequence identity” or “sequence homology” refers to the nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blast program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and intervening integer values. Typically, the percent identities between a disclosed sequence and a claimed sequence are at least 80%, at least 85%, at least 90%, at least 95%, or at least 98%.

As used throughout the disclosure, “substantially identical” refers to having a sequence identity that is 85% or more, for example 90% or more, e.g., 95%, 96%, 97%, 98%, 99%, 99.5%, 99.9%, or 100%, wherein the activity of the composition is unaltered by the modifications in the sequence that result in the difference in sequence identity.

As used throughout the disclosure, an “amino acid mutation” refers to any difference in an amino acid sequence relative to a corresponding parental sequence, e.g., an amino acid substitution, deletion, and/or insertion.

As used throughout the disclosure, the terms “virus vector,” or “viral vector,” refer to a virus particle that functions as a nucleic acid delivery vehicle, and which comprises a nucleic acid (e.g., an expression cassette) packaged within a virion. Exemplary virus vectors of the disclosure include adenovirus vectors, adeno-associated virus vectors (AAVs), lentivirus vectors, and retrovirus vectors.

An “AAV virion” or “AAV virus” or “AAV viral particle” or “AAV vector particle” or “AAV vector” refers to a viral particle comprising at least one AAV capsid polypeptide and an encapsidated polynucleotide (e.g., an AAV vector genome comprising an expression cassette). The polynucleotide may comprise a heterologous nucleic acid (i.e. a polynucleotide other than a wild-type AAV genome, such as an expression cassette to be delivered to a mammalian cell). An AAV vector is often named based on the name (serotype) of the capsid polypeptide. For example, an AAV5 vector indicates that the AAV vector comprises an AAV5 capsid polypeptide.

The terms “vector genome units”, “genome units”, “genome particles (gp)” or “genome copies” (gc) as used in reference to a viral titer, refer to the number of vector genomes encapsidated in the virions, regardless of infectivity or functionality. The number of genome particles in a particular vector preparation can be measured by well understood methods in the art, for example, quantitative PCR of genomic DNA or for example, in Clark et al. (1999) Hum. Gene Ther., 10:1031-1039; Veldwijk et al. (2002) Mol. Ther., 6:272-278.

The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Virol., 62:1963-1973.

An “infectious” virion, virus or viral particle is one comprising a polynucleotide component deliverable into a cell tropic for the viral species. The term does not necessarily imply any replication capacity of the virus. As used herein, an “infectious” virus or viral particle is one that upon accessing a target cell, can infect a target cell, and can express a heterologous nucleic acid in a target cell. Thus, “infectivity” refers to the ability of a viral particle to access a target cell, enter a target cell, and express a heterologous nucleic acid in a target cell. Infectivity can refer to in vitro infectivity or in vivo infectivity. Viral infectivity can be expressed as the ratio of infectious viral particles to total viral particles.

The term “transducing unit” (tu)” as used in reference to a viral titer, refers to the number of infectious recombinant AAV vector particles that result in the production of a functional transgene product as measured in functional assays such as described in, for example, in Xiao et al. (1997) Exp. Neurobiol., 144:113-124; or in Fisher et al. (1996) J. Virol., 70:520-532 (LFU assay).

The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA) or fluorescence-activated cell sorting (FACS).

The term “tropism” refers to the ability of an AAV vector to infect one or more specified cell types, but can also encompass how the vector functions to transduce the cell in the one or more specified cell types; i.e., tropism refers to preferential entry of the AAV virion into certain cell or tissue type(s) and/or preferential interaction with the cell surface that facilitates entry into certain cell or tissue types, optionally and preferably followed by expression (e.g., transcription and, optionally, translation) of sequences carried by the AAV virion in the cell, e.g., for a recombinant virus, expression of the heterologous nucleotide sequence(s).

The term “tropism profile” refers to the pattern of transduction of one or more target cells, tissues and/or organs by an AAV vector described herein.

Unless indicated otherwise, “efficient transduction” or “efficient tropism,” or similar terms, can be determined by reference to a suitable control (e.g., at least 50%, 60%, 70%, 80%, 85%, 90%, 95%, 100%, 110%, 1²⁵%, 150%, 1⁷⁵%, or 200% or more of the transduction or tropism, respectively, of the control). Suitable controls will depend on a variety of factors including the desired tropism profile. Similarly, it can be determined if a capsid and/or virus “does not efficiently transduce” or “does not have efficient tropism” for a target tissue, or similar terms, by reference to a suitable control.

As used throughout the disclosure, “neuronal activity”, “activity of a neuron”, “neuronal firing” and variations and synonyms thereof, refer to the electrical activity resulting from the stimulation or excitation of a neuron. In some embodiments, neuronal activity is measured using automated or manual patch clamp techniques. In some embodiments, determining the activity of a neuron comprises determining the excitatory postsynaptic potential (EPSP), inhibitory postsynaptic potential (IPSP), and/or action potential of the neuron. In some embodiments, the level of activity of a neuron depends on, or is affected by, the excitatory postsynaptic potential (EPSP), inhibitory postsynaptic potential (IPSP), and/or action potential.

As used throughout the disclosure, a “neurological disease” or “neurological disorder” refers to a disease or disorder of the nervous system. In some embodiments, the neurological disease is associated with, caused by, or results from structural, biochemical, and/or electrical abnormalities in the brain, spinal cord, a nerve, or any component of the nervous system.

As used throughout the disclosure, a “symptom” of a disease refers to a physical or mental feature which is regarded as indicating a condition of disease, particularly such a feature that is apparent to the patient. In some embodiments, the symptom is subjectively evaluated by the patient. For example, in some embodiments, the symptom is pain; in some embodiments, the symptom is seizure.

As used throughout the disclosure in relation to the position of an amino acid, the terms “corresponding to” or “correspond to” refer to an amino acid in a first polypeptide sequence that aligns with a given amino acid in a reference polypeptide sequence when the first polypeptide and reference polypeptide sequences are aligned. Alignment is performed by one of skill in the art using software designed for this purpose, for example, Clustal Omega version 1.2.4 with the default parameters for that version.

As used throughout the disclosure, the term “expression cassette” refers to a DNA segment that contains one or more transgene(s) to be transcribed and one or more regulatory element(s) (including, but not limited to, a promoter, an enhancer, an intron, a UTR, and/or a polyA). When introduced into an appropriate host cell, an expression cassette inter alia is capable of directing the cell's machinery to transcribe the transgene into RNA.

As used throughout the disclosure, the term “promoter” refers to one or more nucleic acid control sequences that direct transcription of an operably linked nucleic acid. Promoters may include nucleic acid sequences near the start site of transcription, such as a TATA element. Promoters may also include cis-acting polynucleotide sequences that can be bound by transcription factors. A “constitutive” promoter is a promoter that is active under most environmental and developmental conditions. A promoter may be inducible or repressible. An “inducible” promoter initiates increased level of transcription from the operably linked nucleic acid in response to certain changes in external conditions (e.g., temperature, the concentration of a specific molecule, or oxygen or nutrient level). In some embodiments, the promoter is “tissue-specific”, which causes the operably linked nucleic acid to be expressed at a substantially higher level in a specific cell type(s) or tissues(s) compared to a control cell type or tissue. In some embodiments, the promoter may be neuron-specific and confers substantially greater (e.g., 2-fold, 5-fold, or 10-fold higher) expression of the operably linked nucleic acid in neurons than in non-neuron cells.

As used throughout the disclosure, the term “operably linked” refers to a functional linkage between a nucleic acid expression control sequence (such as a promoter or array of transcription factor binding sites) and a second nucleic acid sequence, wherein the expression control sequence directs transcription of the second nucleic acid sequence.

As used throughout the disclosure, the term “enhancer” refers to a polynucleotide sequence which typically acts on the activity of a promoter in the cis location and thus stimulates the transcription of a gene operably linked to the promoter. Usually, the effect of an enhancer is fairly independent of the position and orientation and therefore it may be positioned in front of or behind a promoter, within an intron or even within a gene coding region. The enhancer may be located in the immediate vicinity of the gene coding region or the promoter, or at a considerable distance from the promoter. It is also possible to have a physical and/or functional overlap with the promoter.

### Overview

The present disclosure provides expression cassettes for expressing of a transgene in target cells. In some embodiments, the target cells are hippocampal neurons. In some embodiments, the target cells are dorsal root ganglion neurons. In some embodiments, the target cells are trigeminal ganglion neurons.

In some embodiments, the expression cassette of the disclose is optimized for one or more of the following properties:
Preferential expression in neurons;
Promoting sustained gene expression;
Expression at sufficient levels to be efficacious without causing serious cell health issues;
Being packaged easily into an AAV vector.

In some embodiments, the expression cassette is further optimized to balance the expression level, efficacy, and toxicity. In some embodiments, the expression cassette of the disclosure allows medium to low level expression of the transgene in a target cell but enables a wider therapeutic window in vivo compared to a control expression cassette that allows higher expression of the transgene (e.g., the corresponding small molecule ligand of the transgene may be administered at a wider range without causing toxicity).

In some embodiments, the expression cassette of the disclosure mediates high level expression of the encoded transgene in dorsal root ganglion (DRG) and/or trigeminal ganglion (TGG) neurons. In some embodiments, the expression cassette of the disclosure enhances the expression of the encoded transgene as compared to a control expression cassette. In some embodiments, the expression cassette of the disclosure is delivered to DRG and/or TGG neurons for treating neuropathic pain. In some embodiments, the expression cassette comprises a polynucleotide encoding an engineered ligand gated ion channel (eLGIC) receptor or a chimeric version thereof.

In some embodiments, the expression cassette mediates higher level expression of the encoded transgene in human dorsal root ganglion and/or trigeminal ganglion neurons in vivo as compared to a control expression cassette. In some embodiments, the expression cassette mediates higher level expression of the encoded transgene in human dorsal root ganglion and/or trigeminal ganglion neurons in vitro as compared to a control expression cassette. In some embodiments, the expression cassette mediates higher level expression of the encoded transgene in human dorsal root ganglion and/or trigeminal ganglion neurons ex vivo as compared to a control expression cassette.

In some embodiments, the expression cassette of the disclosure mediates high level expression of the encoded transgene in hippocampal neurons. In some embodiments, the expression cassette mediates high level expression of the encoded transgene preferentially in the excitatory neurons of the hippocampus. In some embodiments, the expression cassette of the disclosure enhances the expression of the encoded transgene as compared to a control expression cassette. In some embodiments, the expression cassette of the disclosure is delivered to hippocampal neurons for treating focal epilepsy. In some embodiments, the expression cassette comprises a polynucleotide encoding a engineered ligand gated ion channel (eLGIC) receptor or a chimeric version thereof.

In some embodiments, the expression cassette mediates higher level expression of the encoded transgene in hippocampal neurons as compared to a control expression cassette. In some embodiments, the expression cassette mediates higher level expression of the encoded transgene in hippocampal neurons as compared to a control expression cassette.

In some embodiments, the expression cassette mediates higher level expression of the encoded transgene in human hippocampal neurons in vivo as compared to a control expression cassette. In some embodiments, the expression cassette mediates higher level expression of the encoded transgene in human hippocampal neurons in vitro as compared to a control expression cassette. In some embodiments, the expression cassette mediates higher level expression of the encoded transgene in human hippocampal neurons ex vivo as compared to a control expression cassette.

In some embodiments, the expression cassette is comprised in a vector (e.g., a viral vector or non-viral vector). In some embodiments, the expression cassette is comprised within an AAV vector. In some embodiments, the expression cassette is a CRISPR/CAS expression system. In some embodiments, the expression cassette encodes a therapeutic protein or antibody.

The present disclosure provides methods of using the expression cassette or a corresponding vector of the disclosure in a therapeutic treatment regimen, vaccine, or research tool development manner.

The present disclosure also provides methods of using the expression cassette of the disclosure to reduce the amount of total nucleic acid administered to a subject. The method comprises administering less total nucleic acid amount to said subject compared to the amount of nucleic acid administered to said subject when said nucleic acid comprises a control expression cassette.

The present disclosure also provides methods of treating neuropathic pain, such as Peripheral Neuropathy and Trigeminal Neuralgia, in a subject in need thereof, comprising administering an effective amount of a recombinant nucleic acid comprising the expression cassette, wherein the expression cassette comprises a transgene encoding a ligand-gated ion channel or chimeric version thereof.

The present disclosure also provides methods of treating focal epilepsy in a subject in need thereof, comprising administering an effective amount of a recombinant nucleic acid comprising the expression cassette, wherein the expression cassette comprises a transgene encoding a ligand-gated ion channel or chimeric version thereof.

### Expression Cassette

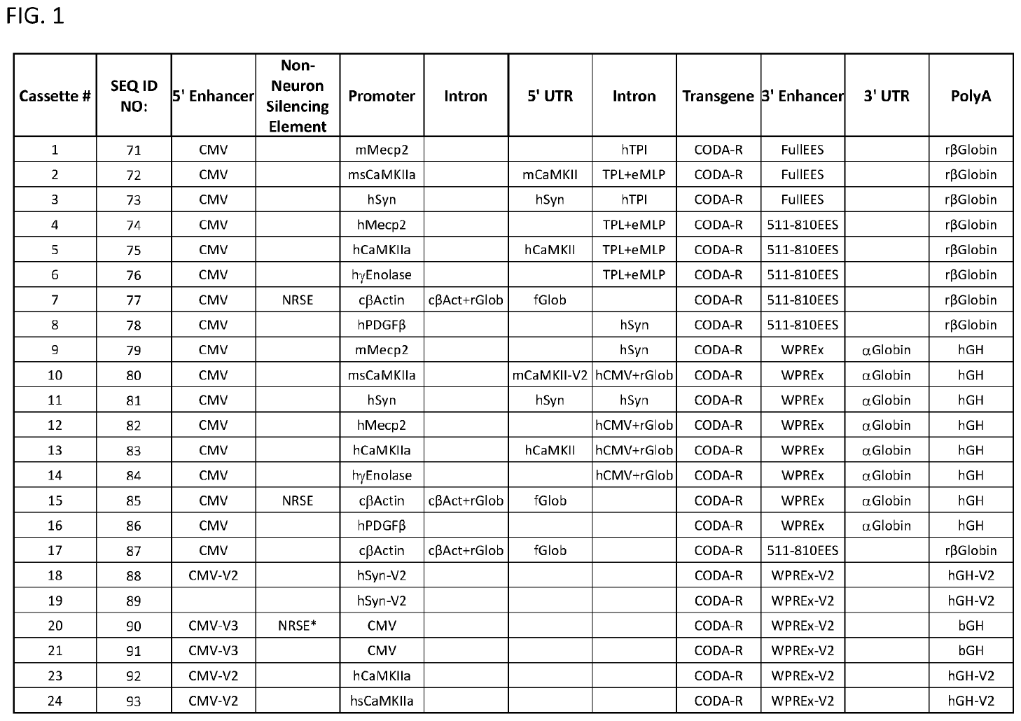

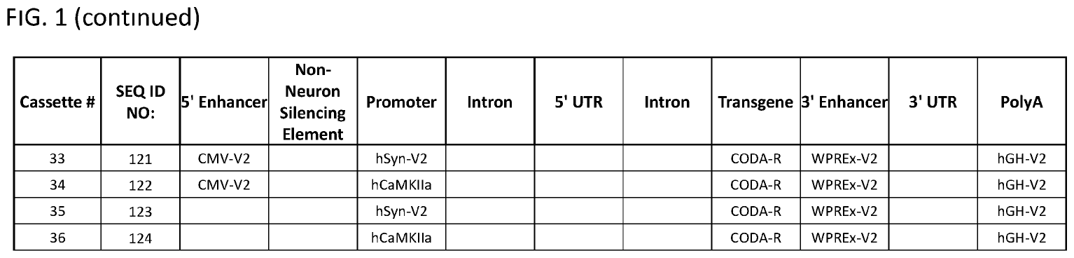

In some embodiments, the expression cassette comprises one or more of the regulatory elements provided in Table 1 below. Exemplary arrangements of the various elements are provided in FIG. 1.

TABLE 1
Non-limiting Examples of Regulatory Elements

| Type | Name of the Regulatory Element | SEQ ID NO: |
|---|---|---|
| 5′ Enhancer | CMV 5′ Enhancer | 37 |
| | CMV-V2 5′ Enhancer | 38 |
| | CMV-V3 5′ Enhancer | 39 |
| Non-Neuron | NRSE | 40 |
| Silencing | | |
| Element | | |
| Promoter | cβActin promoter | 41 |
| | CMV promoter | 42 |
| | hCaMKIIa promoter | 43 |
| | hsCaMKIIa promoter | 44 |
| | hγEnolase promoter | 45 |
| | hMecp2 promoter | 46 |
| | hPDGFβ promoter | 47 |
| | hSyn promoter | 48 |
| | hSyn-V2 promoter | 49 |
| | mMecp2 promoter | 50 |
| | msCaMKIIa promoter | 51 |
| 5′ UTR | fGlob 5′ UTR | 52 |
| | hCaMKII 5′ UTR | 53 |
| | hSyn 5′ UTR | 54 |
| | mCaMKII 5′ UTR | 55 |
| | mCaMKII-V2 5′ UTR | 56 |
| Intron | cβAct + rGlob intron | 57 |
| | hCMV + rGlob intron | 58 |
| | hSyn intron | 59 |
| | hTPI intron | 60 |
| | TPL + eMLP intron | 61 |
| 3′ Enhancer | 511-810EES 3′ Enhancer | 62 |
| | FullEES 3′ Enhancer | 63 |
| | WPREx 3′ Enhancer | 64 |
| | WPREx-V2 3′ Enhancer | 65 |
| 3′ UTR | aGlobin 3′ UTR | 66 |
| PolyA | bGH polyA | 67 |
| | hGH poly A | 68 |
| | hGH-V2 poly A | 69 |
| | rβGlobin poly A | 70 |

### 5′ Enhancer

In some embodiments, the expression cassette of the disclosure comprises a 5′ enhancer.

In some embodiments, the 5′ enhancer comprises or consists of a CMV enhancer. In some embodiments, the CMV enhancer comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 37. In some embodiments, the CMV enhancer comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 37.

In some embodiments, the 5′ enhancer comprises or consists of a CMV-V2 enhancer. In some embodiments, the CMV-V2 enhancer comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 38. In some embodiments, the CMV-V2 enhancer comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 38.

In some embodiments, the 5′ enhancer comprises or consists of a CMV-V3 enhancer. In some embodiments, the CMV-V3 enhancer comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 39. In some embodiments, the CMV-V3 enhancer comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 39.

In some embodiments, the expression cassette of the disclosure does not comprise a 5′ enhancer.

### Non-Neuron Silencing Element

In some embodiments, the expression cassette of the disclosure comprises a non-neuron silencing element, also known as a neuron-restrictive silencer element (NRSE).

NRSEs have been identified in several neuronal genes and confer neuron specificity by silencing transcription in nonneuronal cells. For example, a neuron-restrictive silencer element is present in the promoter of the neuronal nicotinic acetylcholine receptor 02-subunit gene that determines its neuron-specific expression in the nervous system. In some embodiments, such a NRSE may either silence or enhance transcription depending on the cellular context within the nervous system. Studies have shown that certain NRSEs may activate transcription of synthetic promoters when located downstream in the 5′ untranslated region, or at less than 50 bp upstream from the TATA box, but switches to a silencer when located further upstream. More discussion of NRSE may be found at Bessis et al., Proc Natl Acad Sci USA. 1997 May 27; 94(11): 5906-5911, the content of which is incorporated by reference in its entirety for all purposes.

In some embodiments, the NRSE of the disclosure comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 40. In some embodiments, the NRSE comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 40.

In some embodiments, the NRSE is embedded in a promoter of the disclosure. In some embodiments, the NRSE is placed between 30 and 50 base pairs after the TATA box in the expression cassette.

In some embodiments, the expression cassette of the disclosure does not comprise a neuron-restrictive silencer element (NRSE).

### Promoter

In some embodiments, the expression cassette of the disclosure comprises a promoter. In some embodiments, the promoter is operably linked to the transgene of the expression cassette. In some embodiments, the promoter is a tissue-specific promoter. In some embodiments, the promoter is a neuron-specific promoter.

In some embodiments, the promoter comprises or consists of a coActin promoter. In some embodiments, the coActin promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 41. In some embodiments, the coActin promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 41.

In some embodiments, the promoter comprises or consists of a CMV promoter. In some embodiments, the CMV promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 42. In some embodiments, the CMV promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 42.

In some embodiments, the promoter comprises or consists of a hCaMKIIa promoter. In some embodiments, the hCaMKIIa promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 43. In some embodiments, the hCaMKIIa promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 43.

In some embodiments, the promoter comprises or consists of a hsCaMKIIa promoter. In some embodiments, the hsCaMKIIa promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 44. In some embodiments, the hsCaMKIIa promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 44.

In some embodiments, the promoter comprises or consists of a hyEnolase promoter. In some embodiments, the hyEnolase promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 45. In some embodiments, the hyEnolase promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 45.

In some embodiments, the promoter comprises or consists of a hMecp2 promoter. In some embodiments, the hMecp2 promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 46. In some embodiments, the hMecp2 promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 46.

In some embodiments, the promoter comprises or consists of a hPDGFP promoter. In some embodiments, the hPDGFP promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 47. In some embodiments, the hPDGFP promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 47.

In some embodiments, the promoter comprises or consists of a hSyn promoter. In some embodiments, the hSyn promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 48. In some embodiments, the hSyn promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 48.

In some embodiments, the promoter comprises or consists of a hSyn-V2 promoter. In some embodiments, the hSyn-V2 promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 49. In some embodiments, the hSyn-V2 promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 49.

In some embodiments, the promoter comprises or consists of a mMecp2 promoter. In some embodiments, the mMecp2 promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 50. In some embodiments, the mMecp2 promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 50.

In some embodiments, the promoter comprises or consists of a msCaMKIIa promoter. In some embodiments, the msCaMKIIa promoter comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 51. In some embodiments, the msCaMKIIa promoter comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 51.

In some embodiments, the expression cassette of the disclosure does not comprise a promoter.

### 5′ Untranslated Region (5′ UTR)

In some embodiments, the expression cassette of the disclosure comprises a 5′ untranslated region (5′ UTR).

In some embodiments, the 5′ UTR comprises or consists of a fGlob 5′ UTR. In some embodiments, the fGlob 5′ UTR comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 52. In some embodiments, the fGlob 5′ UTR comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 52.

In some embodiments, the 5′ UTR comprises or consists of a hCaMKII 5′ UTR. In some embodiments, the hCaMKII 5′ UTR comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 53. In some embodiments, the hCaMKII 5′ UTR comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 53.

In some embodiments, the 5′ UTR comprises or consists of a hSyn 5′ UTR. In some embodiments, the hSyn 5′ UTR comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 54. In some embodiments, the hSyn 5′ UTR comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 54.

In some embodiments, the 5′ UTR comprises or consists of a mCaMKII 5′ UTR. In some embodiments, the mCaMKII 5′ UTR comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 55. In some embodiments, the mCaMKII 5′ UTR comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 55.

In some embodiments, the 5′ UTR comprises or consists of a mCaMKII-V2 5′ UTR. In some embodiments, the mCaMKII-V2 5′ UTR comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 56. In some embodiments, the mCaMKII-V2 5′ UTR comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 56.

In some embodiments, the expression cassette of the disclosure does not comprise a 5′ UTR.

### Intron

In some embodiments, the expression cassette of the disclosure comprises an intron. In some embodiments, the intron is located between the promoter and the transgene. In some embodiments, the intron is located between the 5′ UTR and the transgene. In some embodiments, the intron is located between the promoter and the 5′ UTR.

In some embodiments, the intron comprises or consists of a cβAct+rGlob intron. In some embodiments, the cβAct+rGlob intron comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 57. In some embodiments, the cβAct+rGlob intron comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 57.

In some embodiments, the intron comprises or consists of a hCMV+rGlob intron. In some embodiments, the hCMV+rGlob intron comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 58. In some embodiments, the hCMV+rGlob intron comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 58.

In some embodiments, the intron comprises or consists of a hSyn intron. In some embodiments, the hSyn intron comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 59. In some embodiments, the hSyn intron comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 59.

In some embodiments, the intron comprises or consists of a hTPI intron. In some embodiments, the hTPI intron comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 60. In some embodiments, the hTPI intron comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 60.

In some embodiments, the intron comprises or consists of a TPL+eMLP intron. In some embodiments, the TPL+eMLP intron comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 61. In some embodiments, the TPL+eMLP intron comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 61.

In some embodiments, the expression cassette of the disclosure does not comprise an intron.

### 3′ Enhancer

In some embodiments, the expression cassette of the disclosure comprises a 3′ enhancer.

In some embodiments, the 3′ enhancer comprises or consists of a 511-810EES 3′ Enhancer. In some embodiments, the 511-810EES 3′ Enhancer comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 62. In some embodiments, the 511-810EES 3′ Enhancer comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 62.

In some embodiments, the 3′ enhancer comprises or consists of a FullEES 3′ Enhancer. In some embodiments, the FullEES 3′ Enhancer comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 63. In some embodiments, the FullEES 3′ Enhancer comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 63.

In some embodiments, the 3′ enhancer comprises or consists of a WPREx 3′ Enhancer. In some embodiments, the WPREx 3′ Enhancer comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 64. In some embodiments, the WPREx 3′ Enhancer comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 64.

In some embodiments, the 3′ enhancer comprises or consists of a WPREx-V2 3′ Enhancer. In some embodiments, the WPREx-V2 3′ Enhancer comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 65. In some embodiments, the WPREx-V2 3′ Enhancer comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 65.

In some embodiments, the expression cassette of the disclosure does not comprise a 3′ enhancer.

### 3′ Untranslated Region (3′ UTR)

In some embodiments, the expression cassette of the disclosure comprises a 3′ untranslated region (3′ UTR).

In some embodiments, the 3′ UTR comprises or consists of an αGlobin 3′ UTR. In some embodiments, the αGlobin 3′ UTR comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 66. In some embodiments, the αGlobin 3′ UTR comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 66.

In some embodiments, the expression cassette of the disclosure does not comprise a 3′ UTR.

### Polyadenylation Sequence (PolyA)

In some embodiments, the expression cassette of the disclosure comprises a polyadenylation sequence (“polyA”, also known as “polyA tail”, “poly-A”, or “poly-A tail”).

In some embodiments, the polyA comprises or consists of a bGH polyA. In some embodiments, the bGH polyA comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 67. In some embodiments, the bGH polyA comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 67.

In some embodiments, the polyA comprises or consists of a hGH polyA. In some embodiments, the hGH polyA comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 68. In some embodiments, the hGH polyA comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 68.

In some embodiments, the polyA comprises or consists of a hGH-V2 polyA. In some embodiments, the hGH-V2 polyA comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 69. In some embodiments, the hGH-V2 polyA comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 69.

In some embodiments, the polyA comprises or consists of a rβGlobin polyA. In some embodiments, the rβGlobin polyA comprises or consists of a polynucleotide sequence having at least 70%, at least 75%, at least 80%, at least 85%, at least 90%, at least 95%, at least 96%, at least 97%, at least 98%, at least 99%, at least 99.5%, or 100% identity to SEQ ID NO: 70. In some embodiments, the rβGlobin polyA comprises or consists of a polynucleotide sequence having up to 1, 2, 3, 4, 5, 6, 7, 8, 9, or 10 nucleotides mutated (substituted, deleted and/or added) as compared to the sequence of SEQ ID NO: 70.

In some embodiments, the expression cassette of the disclosure does not comprise a polyA.

Cassette with Integrated Elements

Exemplary expression cassettes are illustrated in FIG. 1. In some embodiments, the order of the regulatory elements, in 5′ to 3′ order, follows one of the examples provided in Table 2 below.

TABLE 2
Non-limiting Examples of Expression Cassettes

| | 5′ | | | | | | | | | |
|---|---|---|---|---|---|---|---|---|---|---|
| No. | Enhancer | NRSE | Promoter | Intron | 5′ UTR | Intron | Transgene | 3′ Enhancer | 3′ UTR | PolyA |
| 1 | CMV | | mMecp2 | | | hTPI | CODA-R | FullEES | | rβGlobin |
| 2 | CMV | | msCaMKIIa | | mCaMKII | TPL + eMLP | CODA-R | FullEES | | rβGlobin |
| 3 | CMV | | hSyn | | hSyn | hTPI | CODA-R | FullEES | | rβGlobin |
| 4 | CMV | | hMecp2 | | | TPL + eMLP | CODA-R | 511-810EES | | rβGlobin |
| 5 | CMV | | hCaMKIIa | | hCaMKII | TPL + eMLP | CODA-R | 511-810EES | | rβGlobin |
| 6 | CMV | | hγEnolase | | | TPL + eMLP | CODA-R | 511-810EES | | rβGlobin |
| 7 | CMV | NRSE | cβActin | cβAct + rGlob | fGlob | | CODA-R | 511-810EES | | rβGlobin |
| 8 | CMV | | hPDGFβ | | | hSyn | CODA-R | 511-810EES | | rβGlobin |
| 9 | CMV | | mMecp2 | | | hSyn | CODA-R | WPREx | αGlobin | hGH |
| 10 | CMV | | msCaMKIIa | | mCaMKII- | hCMV + rGlob | CODA-R | WPREx | αGlobin | hGH |
| | | | | | V2 | | | | | |
| 11 | CMV | | hSyn | | hSyn | hSyn | CODA-R | WPREx | αGlobin | hGH |
| 12 | CMV | | hMecp2 | | | hCMV + rGlob | CODA-R | WPREx | αGlobin | hGH |
| 13 | CMV | | hCaMKIIa | | hCaMKII | hCMV + rGlob | CODA-R | WPREx | αGlobin | hGH |
| 14 | CMV | | hγEnolase | | | hCMV + rGlob | CODA-R | WPREx | αGlobin | hGH |
| 15 | CMV | NRSE | cβActin | cβAct + rGlob | fGlob | | CODA-R | WPREx | αGlobin | hGH |
| 16 | CMV | | hPDGFβ | | | | CODA-R | WPREx | αGlobin | hGH |
| 17 | CMV | | cβActin | cβAct + rGlob | fGlob | | CODA-R | 511-810EES | | rβGlobin |
| 18 | CMV-V2 | | hSyn-V2 | | | | CODA-R | WPREx-V2 | | hGH-V2 |
| 19 | | | hSyn-V2 | | | | CODA-R | WPREx-V2 | | hGH-V2 |
| 20 | CMV-V3 | NRSE* | CMV | | | | CODA-R | WPREx-V2 | | bGH |
| 21 | CMV-V3 | | CMV | | | | CODA-R | WPREx-V2 | | bGH |
| 23 | CMV-V2 | | hCaMKIIa | | | | CODA-R | WPREx-V2 | | hGH-V2 |
| 24 | CMV-V2 | | hsCaMKIIa | | | | CODA-R | WPREx-V2 | | hGH-V2 |
| 33 | CMV-V2 | | hSyn-V2 | | | | CODA-R | WPREx-V2 | | hGH-V2 |
| 34 | CMV-V2 | | hCaMKIIa | | | | CODA-R | WPREx-V2 | | hGH-V2 |
| 35 | | | hSyn-V2 | | | | CODA-R | WPREx-V2 | | hGH-V2 |
| 36 | | | hCaMKIIa | | | | CODA-R | WPREx-V2 | | hGH-V2 |

“*”: the NRSE element in Example #20 is embedded in, instead of being placed upper stream of, the CMV promoter in this cassette.

In some embodiments, the expression cassette comprises a polynucleotide sequence having at least 70%, at least 75%, at least 80%, at least 85%, at least 90%, at least 95%, at least 96%, at least 97%, at least 98%, at least 99%, at least 99.5%, or 100% identity to any one of SEQ ID NO: 71-93 excluding the region of the transgene (SEQ ID NO: 36). In some embodiments, the expression cassette comprises a polynucleotide sequence having up to 1, 2, 3, 4, 5, 6, 7, 8, 9, or 10 nucleotides mutated (substituted, deleted and/or added) as compared to any one of SEQ ID NO: 71-93 excluding the region of the transgene (SEQ ID NO: 36).

In some embodiments, the expression cassette comprises a polynucleotide sequence having at least 70%, at least 75%, at least 80%, at least 85%, at least 90%, at least 95%, at least 96%, at least 97%, at least 98%, at least 99%, at least 99.5%, or 100% identity to any one of SEQ ID NO: 121-124 excluding the region of the transgene (SEQ ID NO: 32). In some embodiments, the expression cassette comprises a polynucleotide sequence having up to 1, 2, 3, 4, 5, 6, 7, 8, 9, or 10 nucleotides mutated (substituted, deleted and/or added) as compared to any one of SEQ ID NO: 121-124 excluding the region of the transgene (SEQ ID NO: 32).

In some embodiments, the expression cassette comprises a polynucleotide sequence having at least 70%, at least 75%, at least 80%, at least 85%, at least 90%, at least 95%, at least 96%, at least 97%, at least 98%, at least 99%, at least 99.5%, or 100% identity to any one of SEQ ID NO: 71-93 and 121-124. In some embodiments, the expression cassette comprises a polynucleotide sequence having up to 1, 2, 3, 4, 5, 6, 7, 8, 9, or 10 nucleotides mutated (substituted, deleted and/or added) as compared to any one of SEQ ID NO: 71-93 and 121-124.

In some embodiments, the disclosure provides a recombinant nucleic acid comprising the express cassette of the disclosure and an adeno-associated virus (AAV) inverted terminal repeat (ITR) flanking each end of the expression cassette. In some embodiments, the recombinant nucleic acid comprises a 5′ ITR sequence having at least 70%, at least 75%, at least 80%, at least 85%, at least 90%, at least 95%, at least 96%, at least 97%, at least 98%, at least 99%, at least 99.5%, or 100% identity to SEQ ID NO: 94 or 119. In some embodiments, the recombinant nucleic acid comprises a 3′ ITR sequence having at least 70%, at least 75%, at least 80%, at least 85%, at least 90%, at least 95%, at least 96%, at least 97%, at least 98%, at least 99%, at least 99.5%, or 100% identity to SEQ ID NO: 95 or 120.

In some embodiments, the recombinant nucleic acid comprises a polynucleotide sequence having at least 70%, at least 75%, at least 80%, at least 85%, at least 90%, at least 95%, at least 96%, at least 97%, at least 98%, at least 99%, at least 99.5%, or 100% identity to any one of SEQ ID NO: 96-118 and 125-128. In some embodiments, the recombinant nucleic acid comprises a polynucleotide sequence having up to 1, 2, 3, 4, 5, 6, 7, 8, 9, or 10 nucleotides mutated (substituted, deleted and/or added) as compared to any one of SEQ ID NO: 96-118 and 125-128.

In some embodiments, the recombinant nucleic acid comprises a polynucleotide sequence having at least 70%, at least 75%, at least 80%, at least 85%, at least 90%, at least 95%, at least 96%, at least 97%, at least 98%, at least 99%, at least 99.5%, or 100% identity to any one of SEQ ID NO: 96-118 excluding the region of the transgene (SEQ ID NO: 36). In some embodiments, the recombinant nucleic acid comprises a polynucleotide sequence having up to 1, 2, 3, 4, 5, 6, 7, 8, 9, or 10 nucleotides mutated (substituted, deleted and/or added) as compared to any one of SEQ ID NO: 96-118 excluding the region of the transgene (SEQ ID NO: 36).

In some embodiments, the recombinant nucleic acid comprises a polynucleotide sequence having at least 70%, at least 75%, at least 80%, at least 85%, at least 90%, at least 95%, at least 96%, at least 97%, at least 98%, at least 99%, at least 99.5%, or 100% identity to any one of SEQ ID NO: 125-128 excluding the region of the transgene (SEQ ID NO: 32). In some embodiments, the recombinant nucleic acid comprises a polynucleotide sequence having up to 1, 2, 3, 4, 5, 6, 7, 8, 9, or 10 nucleotides mutated (substituted, deleted and/or added) as compared to any one of SEQ ID NO: 125-128 excluding the region of the transgene (SEQ ID NO: 32).

In some embodiments, the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hTPI intron comprising a polynucleotide sequence having at least 90%, at least 95%, at least 96%, at least 97%, at least 98%, at least 99%, at least 99.5%, or 100% identity to SEQ ID NO: 60;
(v) a transgene;
(vi) a FullEES 3′ enhancer comprising a polynucleotide sequence having at least 90%, at least 95%, at least 96%, at least 97%, at least 98%, at least 99%, at least 99.5%, or 100% identity to SEQ ID NO: 63;
(vii) a rβGlobin polyA comprising a polynucleotide sequence having at least 90%, at least 95%, at least 96%, at least 97%, at least 98%, at least 99%, at least 99.5%, or 100% identity to SEQ ID NO: 70.

In some embodiments, the transgene encodes a ligand-gated ion channel comprising a ligand binding domain derived from human a7-nAChR and an ion pore domain derived from a human Glycine receptor. In some embodiments, the expression cassette comprises a polynucleotide sequence having at least 90%, at least 95%, at least 96%, at least 97%, at least 98%, at least 99%, at least 99.5%, or 100% identity to SEQ ID NO: 73.

In some embodiments, the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hSyn intron comprising a polynucleotide sequence having at least 90%, at least 95%, at least 96%, at least 97%, at least 98%, at least 99%, at least 99.5%, or 100% identity to SEQ ID NO: 59;
(v) a transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68.

In some embodiments, the transgene encodes a ligand-gated ion channel comprising a ligand binding domain derived from human a7-nAChR and an ion pore domain derived from a human Glycine receptor. In some embodiments, the expression cassette comprises a polynucleotide sequence having at least 90%, at least 95%, at least 96%, at least 97%, at least 98%, at least 99%, at least 99.5%, or 100% identity to SEQ ID NO: 81.

In some embodiments, the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(iii) a hCaMKII 5′ UTR comprising a polynucleotide sequence having at least 90%, at least 95%, at least 96%, at least 97%, at least 98%, at least 99%, at least 99.5%, or 100% identity to SEQ ID NO: 53;
(iv) a hCMV+rGlob intron comprising a polynucleotide sequence having at least 90%, at least 95%, at least 96%, at least 97%, at least 98%, at least 99%, at least 99.5%, or 100% identity to SEQ ID NO: 58;
(v) a transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68.

In some embodiments, the transgene encodes a ligand-gated ion channel comprising a ligand binding domain derived from human a7-nAChR and an ion pore domain derived from a human Glycine receptor. In some embodiments, the expression cassette comprises a polynucleotide sequence having at least 90%, at least 95%, at least 96%, at least 97%, at least 98%, at least 99%, at least 99.5%, or 100% identity to SEQ ID NO: 83.

In some embodiments, the expression cassette comprises, in 5′ to 3′ order,
(i) a CMV-V2 5′ enhancer comprising a polynucleotide sequence having at least 90%, at least 95%, at least 96%, at least 97%, at least 98%, at least 99%, at least 99.5%, or 100% identity to SEQ ID NO: 38;
(ii) a hSyn-V2 promoter comprising a polynucleotide sequence having at least 90%, at least 95%, at least 96%, at least 97%, at least 98%, at least 99%, at least 99.5%, or 100% identity to SEQ ID NO: 49;
(iii) the transgene;
(iv) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(v) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

In some embodiments, the expression cassette comprises, in 5′ to 3′ order,
(i) a CMV-V2 5′ enhancer comprising a polynucleotide sequence having at least 90%, at least 95%, at least 96%, at least 97%, at least 98%, at least 99%, at least 99.5%, or 100% identity to SEQ ID NO: 38;
(ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(iii) the transgene;
(iv) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(v) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

In some embodiments, the expression cassette comprises, in 5′ to 3′ order,
(i) a hSyn-V2 promoter comprising a polynucleotide sequence having at least 90%, at least 95%, at least 96%, at least 97%, at least 98%, at least 99%, at least 99.5%, or 100% identity to SEQ ID NO: 49;
(ii) the transgene;
(iii) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(iv) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

In some embodiments, the expression cassette comprises, in 5′ to 3′ order,
(i) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(ii) the transgene;
(iii) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(iv) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

### Vector

In some embodiments, the disclosure provides vectors comprising a recombinant nucleic acid, wherein the recombinant nucleic acid comprises the expression cassette of the disclosure.

In some embodiments, the vector is a non-viral vector. In some embodiments, the vector is a plasmid. In some embodiments, the vector is a bacmid.

In some embodiments, the vector is encapsulated in a particle, such as a lipid nanoparticle or an exosome.

In some embodiments, the vector is a viral vector. In some embodiments, the viral vector is derived from a replication-deficient virus. In some embodiments, the viral vector is an adenovirus, a retrovirus (e.g., lentivirus), an adeno-associated virus (AAV), a poxvirus, an alphavirus, a vaccinia virus, or a herpes simplex virus (e.g., HSV-1).

### Adeno-Associated Viruses (AAV) Vector

Adeno-associated virus (AAV), a member of the Parvovirus family, is a small nonenveloped, icosahedral virus with single-stranded linear DNA genomes of about 4.7 kilobases (kb). AAV is assigned to the genus, Dependovirus, because the virus was discovered as a contaminant in purified adenovirus stocks (D. M. Knipe, P. M. Howley, Field's Virology, Lippincott Williams & Wilkins, Philadelphia, ed. Sixth, 2013). In its wild-type state, AAV depends on a helper virus—typically adenovirus—to provide necessary protein factors for replication, as AAV is naturally replication-defective. The 4.7-kb genome of AAV is flanked by two inverted terminal repeats (ITRs) that fold into a hairpin shape important for replication. Being naturally replication-defective and capable of transducing nearly every cell type in the human body, AAV represents an ideal vector for therapeutic use in gene therapy or vaccine delivery. In it's wild-type state, AAV's life cycle includes a latent phase during which AAV genomes, after infection, are site-specifically integrated into host chromosomes and an infectious phase during which, following either adenovirus or herpes simplex virus infection, the integrated genomes are subsequently rescued, replicated, and packaged into infectious viruses. When vectorized, the viral Rep and Cap genes of AAV are removed and provided in trans during virus production, making the ITRs the only viral DNA that remains (A. Vasileva, R. Jessberger, Nature reviews. Microbiology, 3, 837-847 (2005)). Rep and Cap are then replaced with an array of possible transfer vector configurations to perform gene addition or gene targeting. These vectorized AAVs transduce both dividing and non-dividing cells and show robust stable expression in quiescent tissues. A variety of published US applications describe AAV vectors and virions, including U.S. Publication Nos. 2015/0176027, 2015/0023924, 2014/0348794, 2014/0242031, and 2012/0164106; all of which are incorporated by reference herein in their entireties.

Yet, despite the impressive abilities of AAV vectors to transduce a variety of tissue and cell types, there remains a need in the art for AAV vectors with improved transduction of particular neuron cells in terms of both transduction efficiency and desirable tropism. In addition, the method of administration may also affect the locations, amount and types of cells that are transduced. High levels of transduction and the right tropism are needed for delivery to neurons as there are physical limitations to how much AAV can be delivered in a single injection, and the desirable results (e.g., therapeutic results) are related to stimulating or suppressing the activity of the right groups of neurons in the subject.

The present disclosure provides AAV capsid polypeptides that demonstrate significantly improved transduction of a sub-population of neurons in a subject in vivo, in vitro and/or ex vivo.

The genomic organization of all known AAV serotypes is similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VPI, -2 and -3) form the capsid and contribute to the tropism of the virus. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type (wt) AAV infection in mammalian cells the Rep genes are expressed and function in the replication of the viral genome.

In some embodiments, the outer protein “capsid” of the viral vector occurs in nature, e.g., AAV1, AAV2, AAV3, AAV4, AAV5, AAV6, AAV7, AAV8, AAV9, or AAV10. In some embodiments, the capsid is synthetically engineered (e.g., through directed evolution or rational design) to possess certain unique characteristics not present in nature, such as altered tropism, increased transduction efficiency, and/or immune evasion. An example of a rationally designed capsid is the mutation of one or more surface-exposed tyrosine (Y), serine (S), threonine (T), and lysine (K) residues on the VP3 viral capsid polypeptide.

Non-limiting examples of AAV ITRs that may be used in the AAV vectors include ITRs from AAV1, AAV2, AAV3, AAV4, AAV5, AAV6, AAV7, AAV8, AAV9, AAV10, AAV11, AAV12, AAV13, AAV14, AAV15, and AAV16.

AAV vectors comprising two ITRs have a payload capacity of about 4.4 kB. Self-complementary AAV (scAAV) vectors contain a third ITR and package two strands of the recombinant portion of the vector leaving only about 2.1 kB for the polynucleotides contemplated herein. In one embodiment, the AAV vector is an scAAV vector.

Extended packaging capacities that are roughly double the packaging capacity of an AAV (about 9 kB) have been achieved using dual AAV vector strategies. Dual vector strategies useful in producing AAV contemplated herein include, but are not limited to, splicing (trans-splicing), homologous recombination (overlapping), or a combination of the two (hybrid). In the dual AAV trans-splicing strategy, a splice donor (SD) signal is placed at the 3′ end of the 5′-half vector and a splice acceptor (SA) signal is placed at the 5′ end of the 3′-half vector. Upon co-infection of the same cell by the dual AAV vectors and inverted terminal repeat (ITR)-mediated head-to-tail concatemerization of the two halves, trans-splicing results in the production of a mature mRNA and full-size protein (Yan et al., 2000). Trans-splicing has been successfully used to express large genes in muscle and retina (Reich et al., 2003; Lai et al., 2005). Alternatively, the two halves of a large expression cassette contained in dual AAV vectors may contain homologous overlapping sequences (at the 3′ end of the 5′-half vector and at the 5′ end of the 3′-half vector, dual AAV overlapping), which will mediate reconstitution of a single large genome by homologous recombination (Duan et al., 2001). This strategy depends on the recombinogenic properties of the transgene overlapping sequences (Ghosh et al., 2006). A third dual AAV strategy (hybrid) is based on adding a highly recombinogenic region from an exogenous gene (i.e., alkaline phosphatase; Ghosh et al., 2008, Ghosh et al., 2011)) to the trans-splicing vectors. The added region is placed downstream of the SD signal in the 5′-half vector and upstream of the SA signal in the 3′-half vector in order to increase recombination between the dual AAVs.

A “hybrid AAV” refers to an AAV genome packaged with a capsid of a different AAV serotype (and preferably, of a different serotype from the one or more AAV ITRs), and may otherwise be referred to as a pseudotyped AAV. For example, an AAV type 1, 2, 3, 4, 5, 6, 7, 8, 9, 10, 11, 12, 13, 14, 15, or 16 genome may be encapsidated within an AAV type 1, 2, 3, 4, 5, 6, 7, 8, 9, 10, 11, 12, 13, 14, 15, or 16 capsid or variants thereof, provided that the AAV capsid and genome (and preferably, the one or more AAV ITRs) are of different serotypes. In certain embodiments, a pseudotyped AAV particle may be referred to as being of the type “x/y”, where “x” indicates the source of ITRs and “y” indicates the serotype of capsid, for example a 2/5 AAV particle has ITRs from AAV2 and a capsid from AAV5.

A “host cell” includes cells transfected, infected, or transduced in vivo, ex vivo, or in vitro with a recombinant vector or a polynucleotide of the disclosure. Host cells may include virus producing cells and cells infected with viral vectors. In some embodiments, host cells in vivo are infected with viral vector contemplated herein.

High titer AAV preparations can be produced using techniques known in the art, e.g., as described in U.S. Pat. Nos. 5,658,776; 6,566,118; 6,989,264; and 6,995,006; U.S. 2006/0188484; WO98/22607; WO2005/072364; and WO/1999/011764; and Viral Vectors for Gene Therapy: Methods and Protocols, ed. Machida, Humana Press, 2003; Samulski et al., (1989) J. Virology 63, 3822; Xiao et al., (1998) J. Virology 72, 2224; lnoue et al., (1998) J. Virol. 72, 7024. Methods of producing pseudotyped AAV vectors have also been reported (e.g., WO 00/28004), as well as various modifications or formulations of AAV vectors, to reduce their immunogenicity upon in vivo administration (see e.g., WO 01/23001; WO 00/73316; WO 04/1 12727; WO 05/005610; WO 99/06562).

In some embodiments, the AAV vector comprising the AAV capsid polypeptide (or a mutant of the disclosure) contributes to targeted expression of an engineered receptor to a sub-population of cells or neurons in a subject. In some embodiments, the neurons are nociceptors.

In some embodiments, the AAV comprises a self-complementary genome. As defined herein, an AAV comprising a “self-complementary” or “double stranded” genome refers to an AAV which has been engineered such that the coding region of the AAV is configured to form an intra-molecular double-stranded DNA template, as described in McCarty et al. Self-complementary recombinant adeno-associated virus (scAAV) vectors promote efficient transduction independently of DNA synthesis. Gene Therapy. 8 (16): 1248-54 (2001), which is incorporated by reference in its entirety. The present disclosure contemplates the use, in some cases, of an AAV comprising a self-complementary genome because upon infection (such transduction), rather than waiting for cell mediated synthesis of the second strand of the AAV genome, the two complementary halves of scAAV will associate to form one double stranded DNA (dsDNA) unit that is ready for immediate replication and transcription. It will be understood that instead of the full coding capacity found in AAV (4.7-6 kb), AAV comprising a self-complementary genome can only hold about half of that amount (˜2.4 kb).

In some embodiments, the AAV comprises a single stranded genome. As defined herein, a “single standard” genome refers to a genome that is not self-complementary. In some embodiments, the present disclosure relates to single-stranded AAV vectors capable of achieving efficient gene transfer to anterior segment in the mouse eye. See Wang et al. Single stranded adeno-associated virus achieves efficient gene transfer to anterior segment in the mouse eye. PLoS ONE 12(8): e0182473 (2017), the content of which is incorporated by reference in its entirety.

### Capsid Polypeptides

In some embodiments, the present disclosure provides capsid polypeptides that enables the corresponding encapsidated AAV vectors to achieve enhanced transduction and/or tropism for at least a subpopulation of neuron cells. Illustrative capsid polypeptides of the disclosure are listed in Table 3 below:

TABLE 3
Illustrative Capsid Polypeptides

| | Amino Acid | Nucleotide |
|---|---|---|
| Name | Sequence | Sequence |
| AAV2 | SEQ ID NO: 1 | SEQ ID NO: 13 |
| AAV2.5 | SEQ ID NO: 2 | SEQ ID NO: 14 |
| AAV2.5-TV | SEQ ID NO: 3 | SEQ ID NO: 15 |
| AAV2.5-2YF | SEQ ID NO: 4 | SEQ ID NO: 16 |
| AAV2.5-TV2YF | SEQ ID NO: 5 | SEQ ID NO: 17 |
| AAV5 | SEQ ID NO: 6 | SEQ ID NO: 18 |
| AAV6 | SEQ ID NO: 7 | SEQ ID NO: 19 |
| AAV9 | SEQ ID NO: 8 | SEQ ID NO: 20 |
| AAV9-TV | SEQ ID NO: 9 | SEQ ID NO: 21 |
| AAV9-2YF | SEQ ID NO: 10 | SEQ ID NO: 22 |
| AAV9-TV2YF | SEQ ID NO: 11 | SEQ ID NO: 23 |

In some embodiments, the capsid polypeptide of the disclosure is AAV2. In some embodiments, the capsid polypeptide of the disclosure comprises, consists essentially of, or consists of a sequence according to SEQ ID NO: 1. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 1.

In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 1. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 13.

In some embodiments, the capsid polypeptide of the disclosure is AAV2.5. In some embodiments, the capsid polypeptide of the disclosure comprises, consists essentially of, or consists of a sequence according to SEQ ID NO: 2. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 2.

In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 2. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 14.

In some embodiments, the capsid polypeptide of the disclosure is AAV2.5-TV. In some embodiments, the capsid polypeptide of the disclosure comprises an amino acid substitution at the position corresponding to T492 of SEQ ID NO: 2. In some embodiments, the capsid polypeptide of the disclosure comprises an amino acid substitution corresponding to T492V of SEQ ID NO: 2. In some embodiments, the capsid polypeptide of the disclosure comprises, consists essentially of, or consists of a sequence according to SEQ ID NO: 3. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 3, wherein the capsid polypeptide comprise a valine at the position corresponding to V492 of SEQ ID NO: 3. In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 3, wherein the capsid polypeptide comprise a valine at the position corresponding to V492 of SEQ ID NO: 3. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 15.

In some embodiments, the capsid polypeptide of the disclosure is AAV2.5-2YF. In some embodiments, the capsid polypeptide of the disclosure comprises an amino acid substitution at the position corresponding to Y705 and/or Y731 of SEQ ID NO: 2. In some embodiments, the capsid polypeptide of the disclosure comprises an amino acid substitution corresponding to Y705F and/or Y731F of SEQ ID NO: 2. In some embodiments, the capsid polypeptide of the disclosure comprises, consists essentially of, or consists of a sequence according to SEQ ID NO: 4. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 4, wherein the capsid polypeptide comprise a phenylalanine at the position(s) corresponding to F705 and/or F731 of SEQ ID NO: 4. In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 4, wherein the capsid polypeptide comprise a phenylalanine at the position(s) corresponding to F705 and/or F731 of SEQ ID NO: 4. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 16.

In some embodiments, the capsid polypeptide of the disclosure is AAV2.5-TV2YF. In some embodiments, the capsid polypeptide of the disclosure comprises an amino acid substitution at the positions corresponding to T492, Y705 and/or Y731 of SEQ ID NO: 2. In some embodiments, the capsid polypeptide of the disclosure comprises amino acid substitutions corresponding to T492V, Y705F and/or Y731F of SEQ ID NO: 2. In some embodiments, the capsid polypeptide of the disclosure comprises, consists essentially of, or consists of a sequence according to SEQ ID NO: 5. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 5, wherein the capsid polypeptide comprise a valine at the position corresponding to V492 of SEQ ID NO: 5, and wherein the capsid polypeptide comprise a phenylalanine at the position(s) corresponding to F705 and/or F731 of SEQ ID NO: 5. In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 5, wherein the capsid polypeptide comprise a valine at the position corresponding to V492 of SEQ ID NO: 5, and wherein the capsid polypeptide comprise a phenylalanine at the position(s) corresponding to F705 and/or F731 of SEQ ID NO: 5. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 17.

In some embodiments, the capsid polypeptide of the disclosure is AAV5. In some embodiments, the capsid polypeptide of the disclosure comprises, consists essentially of, or consists of a sequence according to SEQ ID NO: 6. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 6. In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 6. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 18.

In some embodiments, the capsid polypeptide of the disclosure is AAV6. In some embodiments, the capsid polypeptide of the disclosure comprises, consists essentially of, or consists of a sequence according to SEQ ID NO: 7. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 7. In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 7. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 19.

In some embodiments, the capsid polypeptide of the disclosure is AAV9. In some embodiments, the capsid polypeptide of the disclosure comprises, consists essentially of, or consists of a sequence according to SEQ ID NO: 8. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 8.

In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 8. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 20.

In some embodiments, the capsid polypeptide of the disclosure is AAV9-TV. In some embodiments, the capsid polypeptide of the disclosure comprises an amino acid substitution at the position corresponding to T492 of SEQ ID NO: 8. In some embodiments, the capsid polypeptide of the disclosure comprises an amino acid substitution corresponding to T492V of SEQ ID NO: 8. In some embodiments, the capsid polypeptide of the disclosure comprises, consists essentially of, or consists of a sequence according to SEQ ID NO: 9. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 9, wherein the capsid polypeptide comprise a valine at the position corresponding to V492 of SEQ ID NO: 9. In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 9, wherein the capsid polypeptide comprise a valine at the position corresponding to V492 of SEQ ID NO: 9. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 21.

In some embodiments, the capsid polypeptide of the disclosure is AAV9-2YF. In some embodiments, the capsid polypeptide of the disclosure comprises an amino acid substitution at the position corresponding to Y705 and/or Y731 of SEQ ID NO: 8. In some embodiments, the capsid polypeptide of the disclosure comprises an amino acid substitution corresponding to Y705F and/or Y731F of SEQ ID NO: 8. In some embodiments, the capsid polypeptide of the disclosure comprises, consists essentially of, or consists of a sequence according to SEQ ID NO: 10. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 10, wherein the capsid polypeptide comprise a phenylalanine at the position(s) corresponding to F705 and/or F731 of SEQ ID NO: 10. In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 10, wherein the capsid polypeptide comprise a phenylalanine at the position(s) corresponding to F705 and/or F731 of SEQ ID NO: 10. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 22.

In some embodiments, the capsid polypeptide of the disclosure is AAV9-TV2YF. In some embodiments, the capsid polypeptide of the disclosure comprises an amino acid substitution at the positions corresponding to T492, Y705 and/or Y731 of SEQ ID NO: 8. In some embodiments, the capsid polypeptide of the disclosure comprises amino acid substitutions corresponding to T492V, Y705F and/or Y731F of SEQ ID NO: 8. In some embodiments, the capsid polypeptide of the disclosure comprises, consists essentially of, or consists of a sequence according to SEQ ID NO: 11. In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or 100% sequence identity to SEQ ID NO: 11, wherein the capsid polypeptide comprise a valine at the position corresponding to V492 of SEQ ID NO: 11, and wherein the capsid polypeptide comprise a phenylalanine at the position(s) corresponding to F705 and/or F731 of SEQ ID NO: 11. In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 11, wherein the capsid polypeptide comprise a valine at the position corresponding to V492 of SEQ ID NO: 11, and wherein the capsid polypeptide comprise a phenylalanine at the position(s) corresponding to F705 and/or F731 of SEQ ID NO: 11. In some embodiments, the capsid polypeptide of the disclosure comprises, consists essentially of, or consists of an amino acid sequence encoded by a nucleotide sequence having at least 80%, at least 85%, at least 90%, at least 95%, at least 96%, at least 97%, at least 98%, at least 99%, at least 99.5%, or 100% sequence identity to SEQ ID NO: 23.

In some embodiments, the AAV vector comprises an AAV capsid polypeptide comprising amino acid mutation at one or more positions corresponding to T492, Y705 and/or, Y731, of AAV9 capsid polypeptide (SEQ ID NO: 8), wherein the AAV capsid polypeptide is of serotype AAV2, AAV2.5, AVV5, AAV6, AAV9, or another AAV serotype. In some embodiments, the one or more positions are two or more positions, two positions, or three positions. In some embodiments, the viral vector comprises an AAV capsid polypeptide comprising one or more amino acid substitutions corresponding to T492V, Y705F and/or Y731F, or any combination thereof, of AAV9 capsid polypeptide (SEQ ID NO: 8), wherein the AAV capsid polypeptide is of serotype AAV2, AAV2.5, AVV5, AAV6, AAV9, or another AAV serotype. In some embodiments, the one or more substitutions are two or more substitutions, two substitutions, or three substitutions.

As used herein, “T492, Y705 and/or Y731” refers to, for example, T492+Y705, T492+Y731, T492+Y705+Y731, or any other possible combinations thereof. Similarly, “T492V, Y705F and/or Y731F” refers to, for example, T492V+Y705F, T492V+Y731F, T492V+Y705F+Y731F, or any other possible combinations thereof.

In some embodiments, the capsid polypeptide of the disclosure comprises, consists essentially of, or consists of an amino acid sequence having at least 80%, at least 85%, at least 90%, at least 95%, at least 96%, at least 97%, at least 98%, at least 99%, at least 99.5%, or at least 99.7% sequence identity to SEQ ID NO: 8, wherein the capsid polypeptide comprises an amino acid substitution at the position corresponding to T492 of SEQ ID NO: 8. In some embodiments, the capsid polypeptide of the disclosure comprises, consists essentially of, or consists of an amino acid sequence having up to 1, 2, 3, 4, 5, 6, 7, 8, 9, or 10 amino acids mutated (substituted, deleted and/or added) as compared to the sequence of SEQ ID NO: 8, wherein the capsid polypeptide comprises an amino acid substitution at the position corresponding to T492 of SEQ ID NO: 8. In some embodiments, the substitution is T492V. In some embodiments, the substitution is T492I. In some embodiments, the substitution is T492L.

In some embodiments, the T492 residue is substituted by a hydrophobic amino acid selected from valine (Val), leucine (Leu), and isoleucine (Ile). In some embodiments, the T492 residue is substituted by a hydrophobic amino acid selected from glycine (Gly), alanine (Ala), valine (Val), leucine (Leu), isoleucine (Ile), proline (Pro), phenylalanine (Phe), methionine (Met), and tryptophan (Trp).

### Neurons and Nerve Fiber

In some embodiments, the disclosure provides methods of delivering a recombinant nucleic acid comprising an expression cassette into a target neuron located in the hippocampus, i.e., a hippocampal neuron.

In some embodiments, the hippocampal neuron is an excitatory neuron. In some embodiments, the expression cassette comprises a CAMKII family promoter (e.g., hCaMKIIa) and causes high and/or selective expression of the encoded transgene in an excitatory hippocampal neuron. In some embodiments, the hippocampal neuron is a Ca2+/calmodulin-dependent protein kinase II (CaMKII) positive excitatory neuron.

In some embodiments, the hippocampal neuron is an inhibitory neuron. In some embodiments, the hippocampal neuron is a GABAergic neuron.

In some embodiments, the disclosure provides methods of delivering a recombinant nucleic acid comprising an expression cassette into a target neuron located in the dorsal root ganglion (or spinal ganglion; also known as a posterior root ganglion). Dorsal root ganglion (DRG) is a cluster of neurons (a ganglion) in a dorsal root of a spinal nerve. The cell bodies of sensory neurons known as first-order neurons are located in the dorsal root ganglia. The axons of dorsal root ganglion neurons are known as afferents. In the peripheral nervous system, afferents refer to the axons that relay sensory information into the central nervous system (i.e. the brain and the spinal cord).

In some embodiments, the disclosure provides methods of delivering a recombinant nucleic acid comprising an expression cassette into a target neuron located in the trigeminal ganglion (or Gasserian ganglion, or semilunar ganglion, or Gasser's ganglion). The trigeminal ganglion (TGG) is a sensory ganglion of the trigeminal nerve (CN V) that occupies a cavity (Meckel's cave) in the dura mater, covering the trigeminal impression near the apex of the petrous part of the temporal bone.

An axon (also known as nerve fiber) is a long, slender projection of a neuron cell in vertebrates that typically conducts electrical impulses known as action potentials away from the nerve cell body. Each neuron has only one axon. Neuron cells can thus be classified by their axons (nerve fibers).

Three general classes (Groups A, B and C) of nerve fibers are classified by Erlanger and Gasser. Group A nerve fibers are heavily myelinated, group B nerve fibers are moderately myelinated, and group C nerve fibers are unmyelinated.

There are four subdivisions of group A nerve fibers (A fibers): alpha (α), beta (β), gamma (γ), and delta (δ). These subdivisions have different amounts of myelination and axon thickness and therefore transmit signals at different speeds. Type Aa fibers include the type Ia and type Ib sensory fibers of the alternative classification system, and are the fibers from muscle spindle endings and the Golgi tendon, respectively. Type A3 fibers, and type Ay, are the type II afferent fibers from stretch receptors. Type A3 fibers from the skin are mostly dedicated to touch. Type Aδ fibers are the afferent fibers of nociceptors. Aδ fibers carry information from peripheral mechanoreceptors and thermoreceptors to the dorsal horn of the spinal cord. Aδ fibers serve to receive and transmit information primarily relating to acute pain (sharp, immediate, and relatively short-lasting). This type of pain can result from several classifications of stimulants: temperature-induced, mechanical, and chemical. This can be part of a withdrawal reflex-initiated by the Aδ fibers in the reflex arc of activating withdrawal responses. Aδ fibers carry cold, pressure, and acute pain signals; because they are thin (2-5 m in diameter) and myelinated, they send impulses faster than unmyelinated C fibers, but more slowly than other, more thickly myelinated group A nerve fibers. Their conduction velocities are moderate.

The group B nerve fibers (B fibers) are axons, which are moderately myelinated, which means less myelinated than group A nerve fibers, and more myelinated than group C nerve fibers. They are usually general visceral afferent fibers and preganglionic nerve fibers of the autonomic nervous system.

The group C nerve fibers (C fibers) are unmyelinated and have a small diameter and low conduction velocity. Group C fibers include postganglionic fibers in the autonomic nervous system (ANS), and nerve fibers at the dorsal roots (IV fiber). These fibers carry sensory information. Damage or injury to these nerve fibers causes neuropathic pain.

The peripheral terminal of the mature nociceptor is where the stimuli are detected and transduced into electrical energy. When the electrical energy reaches a threshold value, an action potential is induced and driven towards the central nervous system (CNS). This leads to the train of events that allows for the conscious awareness of pain. The sensory specificity of nociceptors is established by the high threshold only to particular features of stimuli. Only when the high threshold has been reached by either chemical, thermal, or mechanical environments are the nociceptors triggered. The majority of nociceptors are classified by which of the environmental modalities they respond to. Some nociceptors respond to more than one of these modalities and are consequently designated polymodal. Other nociceptors respond to none of these modalities (although they may respond to stimulation under conditions of inflammation) and are referred to as sleeping or silent.

Nociceptors have two different types of axons. The first are the Aδ fiber axons. They are myelinated and can allow an action potential to travel at a rate of about 20 meters/second towards the CNS. The other type is the more slowly conducting C fiber axons. These only conduct at speeds of around 2 meters/second. This is due to the light or non-myelination of the axon. As a result, pain comes in two phases. The first phase is mediated by the fast-conducting Aδ fibers and the second part due to (Polymodal) C fibers. The pain associated with the Aδ fibers can be associated to an initial extremely sharp pain. The second phase is a more prolonged and slightly less intense feeling of pain as a result of the acute damage. If there is massive or prolonged input to a C fiber, there is a progressive build up in the spinal cord dorsal horn; this phenomenon is similar to tetanus in muscles but is called wind-up. If wind-up occurs there is a probability of increased sensitivity to pain.

Different classes of nerve fibers can be differentiated by biomarkers such as Neurofilament 200 (NF200 also known as neurofilament heavy polypeptide), Calcitonin Gene-Related Peptide (CGRP) and isolectin B4 (IB4). Aα and Aβ fibers are typically NF200 positive but IB4 negative. Aδ fibers are typically positive for both NF200 and IB4. C fibers are typically IB4 positive but NF200 negative. Accordingly, NF200+ neurons mostly comprise a heavily myelinated Aα or moderately myelinated Aβ fiber. In some cases, these neurons mediate signals for light touch & proprioception. But some NF200+ neurons may also mediate signals for nociception. IB4+ neurons mostly comprise an unmyelinated C fiber or an Aδ fiber for nociception. And CGRP+ neurons mostly comprise an unmyelinated C fiber or a lightly myelinated Aδ fiber for nociception.

### Properties of the Expression Cassettes

In some embodiments, the expression cassettes of the present disclosure mediate higher level expression of the encoded transgene in one or more neurons as compared to a control expression cassette (e.g., a control with tissue specific expression). In some embodiments, the control expression cassette comprises all the regulatory elements of expression cassette No. 18 in Table 2, which comprises a hSyn-V2 promoter for neuron-specific expression. In some embodiments, such a control expression cassette comprises the polynucleotide sequence of SEQ ID NO: 88 excluding the sequence of the transgene (SEQ ID NO: 36). In some embodiments, the expression cassettes of the present disclosure mediate comparable or higher level expression of the encoded transgene in one or more neurons as compared to a control expression cassette with ubiquitous, rather than tissue-specific, transgene expression. In some embodiments, the control expression cassette comprises all the regulatory elements of expression cassette No. 17 in Table 2, which comprises the CAG (CMV enhancer, coActin promoter, and rβActin-rGlob intron) regulatory element for strong and ubiquitous expression in eukaryote cells. In some embodiments, such a control expression cassette comprises the polynucleotide sequence of SEQ ID NO: 87 excluding the sequence of the transgene (SEQ ID NO: 36). A skilled person would readily recognize the proper control used in each case, and typically the control expression cassette would have the same transgene polynucleotide sequence as that of the expression cassette in comparison.

Expression level of transgene can be measured by techniques known in the art, including, for example, ELISA, ddPCR and/or immunofluorescence analysis, including those described in the Examples section of the disclosure, as well as other methods known in the art.

In some embodiments, the expression cassette mediates higher level expression of the encoded transgene in one or more target neurons as compared to the control expression cassette (e.g., a control with tissue specific expression). In some embodiments, the transgene expression level is increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 2-fold, about 3-fold, about 4-fold, about 5-fold, about 7-fold, about 10-fold, about 20-fold, about 30-fold, about 40-fold, about 50-fold, about 70-fold, or about 100-fold, including all ranges and subranges therebetween, as compared to that of the control expression cassette. In some embodiments, the transgene expression level is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2-fold, at least 3-fold, at least 4-fold, at least 5-fold, at least 7-fold, at least 10-fold, at least 20-fold, at least 30-fold, at least 40-fold, at least 50-fold, at least 70-fold, or at least 100-fold, including all ranges and subranges therebetween, as compared to that of the control expression cassette. In some embodiments, the control expression cassette comprises a polynucleotide sequence having at least 90%, at least 95%, at least 99%, or 100% identity to SEQ ID NO: 88 (Expression Cassette No. 18 in Table 2) excluding the sequence of the transgene.

In some embodiments, the expression cassette mediates comparable or higher level expression of the encoded transgene in one or more target neurons as compared to the control expression cassette with ubiquitous, rather than tissue-specific, transgene expression. In some embodiments, the transgene expression level is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 2-fold, at least 3-fold, at least 4-fold, at least 5-fold, at least 7-fold, at least 10-fold, at least 20-fold, at least 30-fold, at least 40-fold, at least 50-fold, at least 70-fold, or at least 100-fold, including all ranges and subranges therebetween, of the transgene expression level of the control expression cassette. In some embodiments, the control expression cassette comprises a polynucleotide sequence having at least 90%, at least 95%, at least 99%, or 100% identity to SEQ ID NO: 87 (Expression Cassette No. 17 in Table 2) excluding the sequence of the transgene.

In some embodiments, the transgene expression level refers to mRNA expression level. In some embodiments, the transgene expression level refers to protein expression level.

In some embodiments, the target neuron is a hippocampal neuron. In some embodiments, the target neuron is an excitatory neuron.

In some embodiments, the target neuron is a DRG or TGG neuron.

In some embodiments, the target neuron comprises an A fiber. In some embodiments, the target neuron comprises an Aα or Aβ fiber. In some embodiments, the target neuron comprises an Aδ fiber. In some embodiments, the target neuron comprises a C fiber.

In some embodiments, the target neuron comprises a nerve fiber that is NF200 positive and IB4 negative. In some embodiments, the target neuron comprises a nerve fiber that is NF200 positive and IB4 positive. In some embodiments, the target neuron comprises a nerve fiber that is NF200 negative and IB4 positive. In some embodiments, the target neuron comprises a nerve fiber that is CGRP positive.

### Transgene

In some embodiments, the expression cassette of the disclosure comprises a transgene. In some embodiments, the transgene encodes a protein, a peptide, a miRNA, a siRNA, or a gRNA. In some embodiments, the transgene encodes two or more biomolecules (e.g., proteins) separated by an internal ribosome entry site, allowing co-expression of the two or more biomolecules.

In some embodiments, the expression cassette of the disclosure comprises a transgene encoding a receptor. In some embodiments, the receptor is an engineered ligand-gated ion channel (eLGIC) or a chimeric version thereof. In some embodiments, the engineered LGIC can be activated by a small molecule ligand. In some embodiments, administration of the expression cassette encoding the eLGIC delivers it into a sub-population of neuron cells in a subject and causes expression of the eLGIC within the neurons. In some embodiments, expression of the eLGIC allows the activities of these neurons to be modulated by administration of the corresponding small molecule ligand.

In some embodiments, the transgene is operably linked to one or more control/regulatory elements directing its transcription or expression thereof. Control elements (i.e., regulatory elements) may include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Exemplary regulatory elements are described in the present disclosure.

Such control elements can comprise control sequences normally associated with the selected gene (e.g., endogenous cellular control elements). Alternatively, heterologous control sequences can be employed. Useful heterologous control sequences generally include those derived from sequences encoding mammalian or viral genes. Examples include, but are not limited to, the SV40 early promoter, mouse mammary tumor virus long terminal repeat (LTR) promoter; adenovirus major late promoter (Ad MLP); a herpes simplex virus (HSV) promoter, an endogenous cellular promoter heterologous to the gene of interest, a cytomegalovirus (CMV) promoter such as the CMV immediate early promoter region (CMV-IE), a rous sarcoma virus (RSV) promoter, synthetic promoters, hybrid promoters, and the like. In addition, sequences derived from nonviral genes, such as the murine metallothionein gene, can also be used.

In some embodiments, a cell type-specific or a tissue-specific promoter can be operably linked to the transgene, allowing for selectively or preferentially producing the corresponding gene product encoded by the transgene in a particular cell type(s) or tissue(s). In some embodiments, an inducible promoter is operably linked to the transgene.

In some embodiments, the expression cassette encoding the transgene may be delivered to a neuron using a non-viral method (for example, using a non-viral vector, a synthetic nanoparticle, etc.).

Engineered Ligand-Gated Ion Channel (eLGIC)

The present disclosure describes receptors, their mutants, and methods for their use in the treatment of focal epilepsy and/or neuropathic pain, such as Peripheral Neuropathy and Trigeminal Neuralgia. In some embodiments, the receptor is an engineered ligand-gated ion channel (LGIC). In some embodiments, the receptor is a chimeric eLGIC.

In some embodiments, the receptor is an engineered receptor (e.g., an eLGIC). The term “engineered receptor” is used herein to refer to a receptor that has been experimentally altered such that it is physically and/or functionally distinct from a corresponding parental receptor. In some embodiments, the parental receptor is a wild-type receptor. The term “wild-type receptor” is used herein to refer to a receptor having a polypeptide sequence that is identical to the polypeptide sequence of a protein found in nature. Wild-type receptors include receptors that naturally occur in humans as well as orthologs that naturally occur in other eukaryotes, e.g., protist, fungi, plants or animals, for example yeast, insects, nematodes, sponge, mammals, non-mammalian vertebrates. In some embodiments, the parental receptor is a non-native receptor; that is, it is a receptor that does not occur in nature, for example, a receptor that is engineered from a wild-type receptor. For example, a parental receptor may be an engineered receptor comprising one or more subunits from one wild-type receptor with one or more subunits from a second wild-type receptor. The resulting proteins are therefore comprised of subunits from two or more wild-type receptors. Therefore, in some embodiments, the parental receptor is a chimeric receptor. Engineered receptors of the present disclosure include, for example, parental receptor mutants, and switch receptors.

In some embodiments, an engineered receptor of the present disclosure comprises at least one amino acid mutation relative to the corresponding parental receptor, e.g., one or more mutations in one or more domains of a wild-type receptor. In some embodiments, the mutation is an amino acid substitution. In some embodiments, the engineered receptor shares a sequence identity of about 99%, about 98%, about 95%, about 90%, about 85%, about 80%, about 70%, about 60%, about 50%, or less with the corresponding parental receptor, inclusive of all values and subranges that lie therebetween. In some embodiments, the parental receptor mutant has a sequence identity of 85% or more with the corresponding parental receptor, e.g., 90% or more or 95% or more, for example, about 96%, about 97%, about 98% or about 99% identity with the corresponding parental receptor, inclusive of all values and subranges that lie therebetween. In some embodiments, an engineered receptor (e.g., a parental receptor mutant) is generated by error prone PCR.

In some embodiments, the ligand binding domain (LBD) of the engineered receptor of the disclosure comprises at least one amino acid mutation relative to the corresponding ligand binding domain of the parental receptor, e.g., one or more mutations in the ligand binding domain of a wild-type receptor. In some embodiments, the mutation is an amino acid substitution. In some embodiments, the ligand binding domain of the engineered receptor has a sequence identity of 85% or more with the corresponding ligand binding domain of the parental receptor, e.g., 90% or more or 95% or more, for example, about 85%, about 90%, about 95%, about 96%, about 97%, about 98%, about 99% identity, or 100% identity with the corresponding ligand binding domain of the parental receptor, inclusive of all values and subranges that lie therebetween. In some embodiments, the ligand binding domain of the engineered receptor shares a sequence identity of at least 85%, at least 90%, at least 95%, at least 96%, at least 97%, at least 98% or at least 99% identity with the corresponding ligand binding domain of the parental receptor, inclusive of all values and subranges that lie therebetween.

In some embodiments, the ion pore domain (IPD) of the engineered receptor of the disclosure comprises at least one amino acid mutation relative to the corresponding ion pore domain of the parental receptor, e.g., one or more mutations in the ion pore domain of a wild-type receptor. In some embodiments, the mutation is an amino acid substitution. In some embodiments, the ion pore domain of the engineered receptor has a sequence identity of 85% or more with the corresponding ion pore domain of the parental receptor, e.g., 90% or more or 95% or more, for example, about 85%, about 90%, about 95%, about 96%, about 97%, about 98%, about 99% identity, or 100% identity with the corresponding ion pore domain of the parental receptor, inclusive of all values and subranges that lie therebetween. In some embodiments, the ion pore domain of the engineered receptor shares a sequence identity of at least 85%, at least 90%, at least 95%, at least 96%, at least 97%, at least 98% or at least 99% identity with the corresponding ion pore domain of the parental receptor, inclusive of all values and subranges that lie therebetween.

In some embodiments, the amino acid mutation is a loss-of-function amino acid mutation relative to a corresponding parental receptor. “Loss-of-function” amino acid mutations refer to one or more mutations that reduce, substantially decrease, or abolish the function of the engineered receptor relative to the parental receptor, for example by reducing the binding of an endogenous ligand to an engineered receptor relative to the binding of endogenous ligand to the parental receptor, or by reducing the activity of signaling pathway(s) downstream of the engineered receptor that are typically activated in response to the binding of a ligand to the corresponding parental receptor. In some embodiments, the mutation is an amino acid substitution.

In some embodiments, the amino acid mutation is a gain-of-function amino acid mutation relative to a corresponding parental receptor. “Gain-of-function” amino acid mutations refer to one or more mutations that modify the function of the engineered receptor relative to the parental receptor, for example by altering or enhancing the affinity of an engineered receptor for a ligand relative to the binding of endogenous ligand to the parental receptor, or by altering or enhancing the activity of the signaling pathways that are activated in response to the binding of a ligand to an engineered receptor relative to the binding of the endogenous ligand to the corresponding parental receptor. In some embodiments, a gain-of-function mutation results in an increased affinity of the engineered receptor for a ligand. In particular embodiments, a gain-of-function mutation results in an increased affinity of the engineered receptor for an agonist ligand. In some embodiments, a gain-of-function mutation results in an antagonist ligand acting as an agonist ligand upon binding to the engineered receptor (e.g., results in the activation of agonist signaling pathways instead of antagonist signaling pathways). In some embodiments, a gain-of-function mutation results in a modulator ligand acting as an agonist ligand upon binding to the engineered receptor. In some embodiments, the mutation is an amino acid substitution.

In some embodiments, the subject engineered receptor of the present disclosure, or the ligand binding domain and/or the ion pore domain thereof, comprises one or more loss-of-function amino acid mutations and one or more gain-of-function amino acid mutations relative to a corresponding parental receptor. In some embodiments, the mutation is an amino acid substitution.

In some embodiments, the loss of function mutation and the gain of function mutation are at the same residue, i.e. they are the same mutation. In other embodiments, the loss of function mutation and the gain of function mutation are mutations at different amino acid residues. In some embodiments, the mutation is an amino acid substitution. In some embodiments, the subject engineered receptor (or the ligand binding domain and/or the ion pore domain thereof) comprising the loss of function mutation and/or gain of function mutation shares a sequence identity of about 99%, about 98%, about 95%, about 90%, about 85%, about 80%, about 70%, about 60%, about 50%, including all ranges and subranges therebetween, or less with the corresponding parental receptor, e.g., wild type receptor or non-native receptor (or the ligand binding domain and/or the ion pore domain thereof). In some embodiments, the subject engineered receptor (or the ligand binding domain and/or the ion pore domain thereof) shares a sequence identity of 85% or more with the corresponding parental receptor (or the ligand binding domain and/or the ion pore domain thereof), for example 85%, 90%, or 95% or more sequence identity, in some instances 96%, 97%, 98% or more sequence identity, e.g., 99% or 99.5% or more sequence identity, inclusive of all values and subranges that lie therebetween.

In some aspects, engineered receptors of the present disclosure include receptors produced by the combination of one or more amino acid sequences, e.g., subunits, derived from one wild-type receptor with one or more amino acid sequences, e.g., subunits, derived from a second wild-type receptor. In other words, the engineered receptor comprises amino acid sequences that are heterologous to one another, where by “heterologous”, it is meant not occurring together in nature. Such receptors are referred to herein as “chimeric receptors”. In some embodiments, chimeric receptors serve as parental receptors from which an engineered receptor of the present disclosure is generated. In some embodiments, the chimeric receptor comprises a ligand binding domain from a first LGIC and an ion pore domain from a second LGIC.

In some embodiments, a parental receptor mutant demonstrates increased affinity for an agonist ligand. In some embodiments, a ligand that functions as an antagonist or modulator when binding to a wild type receptor functions as an agonist when binding to a parental receptor mutant.

In some embodiments, the engineered receptor is a “ligand-gated ion channel” or LGIC. An LGIC refers to a large group of transmembrane proteins that allow passage of ions upon activation by a specific ligand. LGIC are composed of at least two domains: a ligand binding domain and a transmembrane ion pore domain. Ligand binding to an LGIC results in activation of the LGIC and opening of the ion pore. Ligand binding causes a drastic change in the permeability of the channel to a specific ion or ions; effectively no ions can pass through the channel when it is inactive or closed but up to 107 ions/second can pass through upon ligand binding. In some embodiments, LGICs respond to extracellular ligands (e.g., neurotransmitters) and facilitate an influx of ions into the cytosol. In some embodiments, LGICs respond to intracellular ligands (e.g., nucleotides such at ATP and signaling intermediates such as PIP2) and facilitate an efflux of ions from the cytosol into the extracellular environment. Importantly, activation of LGIC results in the transport of ions across the cellular membrane (e.g., Ca2+, Na+, K+, Cl−, etc.) and does not result in the transport of the ligand itself.

LGIC receptors are comprised of multiple subunits and can be either homomeric receptors or heteromeric receptors. A homomeric receptor is comprised of subunits that are all the same type. A heteromeric receptor is comprised of subunits wherein at least one subunit is different from at least one other subunit comprised within the receptor. For example, the glycine receptor is comprised of 5 subunits of which there are two types: α-subunits, of which there are four isoforms (α1-α4) and β-subunits, of which there is a single known isoform. An exemplary homomeric GlyR is a GlyR comprised of 5 α1-GlyR subunits. Similarly, a homomeric GABAA receptor may be comprised of 03-GABAA subunits, and an nAchR receptor may be comprised of α7-nAchR subunits. An exemplary heteromeric GlyR may be comprised of one or more α-subunits and one or more of β-subunits (e.g., an α1β-GlyR). Subunits of example LGIC receptors are shown in Table 4.

TABLE 4
LGIC Receptors and Subunits

| Receptor | Subunits | Subunit Isoforms |
|---|---|---|
| GlyR | GLRA1 | |
| | GLRA2 | |
| | GLRA3 | |
| | GLRA4 | |
| | GLRB | |
| 5HT₃ | 5-HT3A | |
| | 5-HT3B | |
| | 5-HT3C | |
| | 5-HT3D | |
| | 5-HT3E | |
| nAChR | α | α1 |
| | | α2 |
| | | α3 |
| | | α4 |
| | | α5 |
| | | α6 |
| | | α7 |
| | | α8 |
| | | α9 |
| | | α10 |
| | β | β1 |
| | | β2 |
| | | β3 |
| | | β4 |
| | γ | γ |
| | δ | δ |
| | ε | ε |
| GABA_{A} | α | α1 |
| | | α2 |
| | | α3 |
| | | α4 |
| | | α5 |
| GABA_{A} | α | α6 |
| | β | β1 |
| | | β2 |
| | | β3 |
| | γ | γ1 |
| | | γ2 |
| | | γ3 |
| | δ | δ |
| | ε | ε |
| | π | π |
| | ρ | ρ1 |
| | | ρ2 |
| | | ρ3 |
| P2X | P2X1 | |
| | P2X2 | |
| | P2X3 | |
| | P2X4 | |
| | P2X5 | |
| | P2X6 | |
| | P2X7 | |
| KCNQ | α | Kᵥα1 |
| | | Kᵥα2 |
| | | Kᵥv3 |
| | | Kᵥα4 |
| | | Kᵥα5 |
| | | Kᵥα6 |
| | | Kᵥα7 |
| | | Kᵥα8 |
| | | Kᵥα9 |
| | | Kᵥα10 |
| | | Kᵥα11 |
| | | Kᵥα12 |
| | β | Kᵥβ1 |
| | | Kᵥβ2 |
| | | Kᵥβ3 |
| | | minK |
| | | MiRP1 |
| | | MiRP2 |
| | | MiRP3 |
| | | KCNE1-like |
| | | KCNIP1 |
| | | KCNIP2 |
| | | KCNIP3 |
| | | KCNIP4 |

Illustrative examples of families of LGICs suitable for use in particular embodiments include, but are not limited to Cys-loop receptors such as Glycine receptors (GlyR), serotonin receptors (e.g., 5-HT3 receptors), λ-Aminobutyric Acid A (GABA-A) receptors, and Nicotinic acetylcholine receptors (nAchR); as well as Acid-sensing (proton-gated) ion channels (ASICs), Epithelial sodium channels (ENaC), Ionotropic glutamate receptors, IP3 receptor, P2X receptors, the Ryanodine receptor, and Zinc activated channels (ZAC).

Specific non-limiting examples of LGICs that are suitable for use with the methods described herein include: HTR3A; HTR3B; HTR3C; HTR3D; HTR3E; ASIC1; ASIC2; ASIC3; SCNN1A; SCNN1B; SCNN1D; SCNN1G; GABRA1; GABRA2; GABRA3; GABRA4; GABRA5; GABRA6; GABRB1; GABRB2; GABRB3; GABRG1; GABRG2; GABRG3; GABRD; GABRE; GABRQ; GABRP; GABRR1; GABRR2; GABRR3; GLRA1; GLRA2; GLRA3; GLRA4; GLRB; GRIA1; GRIA2; GRIA3; GRIA4; GRID1; GRID2; GRIK1; GRIK2; GRIK3; GRIK4; GRIK5; GRIN1; GRIN2A; GRIN2B; GRIN2C; GRIN2D; GRIN3A; GRIN3B; ITPR1; ITPR2; ITPR3; CHRNA1; CHRNA2; CHRNA3; CHRNA4; CHRNAS; CHRNA6; CHRNA7; CHRNA9; CHRNA10; CHRNB1; CHRNB2; CHRNB3; CHRNB4; CHRNG; CHRND; CHRNE; P2RX1; P2RX2; P2RX3; P2RX4; P2RX5; P2RX6; P2RX7; RYR1; RYR2; RYR3; and ZACN.

Non-limiting examples of sequences of wild-type LGIC receptor that find use in the generation of engineered receptors of the present disclosure include the following. In the sequences, the signal peptide is italicized, the ligand binding domain is bolded, and the ion pore domain is underlined:

In some embodiments, the wild-type LGIC receptor is a human nicotinic cholinergic receptor alpha 7 subunit (α7-nAchR) (GenBank Accession No. NP_000737.1, SEQ ID NO: 25), encoded by the CHRNA7 gene (GenBank Accession No. NM_000746.5):

```
(SEQ ID NO: 25)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
                130 140 150 160 170 180
RFDATFHTNV LVNSSGHCQY LPPGIFKSSC YIDVRWFPFD VQHCKLKFGS WSYGGWSLDL
                190 200 210 220 230 240
QMQEADISGY IPNGEWDLVG IPGKRSERFY ECCKEPYPDV TFTVTMRRRT LYYGLNLLIP
                250 260 270 280 290 300
CVLISALALL VELLPADSGE KISLGITVLL SLTVEMLLVA EIMPATSDSV PLIAQYFAST
                310 320 330 340 350 360
MIIVGLSVVV TVIVLQYHHH DPDGGKMPKW TRVILLNWCA WELRMKRPGE DKVRPACQHK
                370 380 390 400 410 420
QRRCSLASVE MSAVAPPPAS NGNLLYIGER GLDGVHCVPT PDSGVVCGRM ACSPTHDEHL
                430 440 450 460 470 480
LHGGQPPEGD PDLAKILEEV RYIANRERCQ DESEAVCSEW KFAACVVDRL CLMAFSVETI
                490 500 502
ICTIGILMSA PNFVEAVSKD FA.
```

In some embodiments, the wild-type LGIC receptor is a human alpha 1 glycine receptor (GlyRα1) (GenBank Accession No. NP_001139512.1, SEQ ID NO: 26), encoded by the GLRA1 gene (GenBank Accession No. NM_001146040.1):

```
(SEQ ID NO: 26)
                10 20 30 40 50 60
MYSENTLRLY LWETIVEFSL AASKEAEAAR SAPKPMSPSD FLDKLMGRTS GYDARIRPNF
                70 80 90 100 110 120
KGPPVNVSCN IFINSFGSIA ETTMDYRVNI FLRQQWNDPR LAYNEYPDDS LDLDPSMLDS
                130 140 150 160 170 180
IWKPDLFFAN EKGAHFHEIT TDNKLLRISR NGNVLYSIRI TLTLACPMDL KNFPMDVQTC
                190 200 210 220 230 240
IMQLESFGYT MNDLIFEWQE QGAVQVADGL TLPQFILKEE KDLRYCTKHY NTGKFTCIEA
                250 260 270 280 290 300
RFHLERQMGY YLIQMYIPSL LIVILSWISF WINMDAAPAR VGLGITTVLT MTTQSSGSRA
                310 320 330 340 350 360
SLPKVSYVKA IDIWMAVCLL FVFSALLEYA AVNEVSRQHK ELLRERRKRR HHKSPMLNLF
                370 380 390 400 410 420
QEDEAGEGRF NFSAYGMGPA CLQAKDGISV KGANNSNTTN PPPAPSKSPE EMRKLFIQRA
                430 440 450 457
KKIDKISRIG FPMAFLIENM FYWIIYKIVR REDVHNQ.
```

In some embodiments, the wild-type LGIC receptor is a human alpha 2 glycine receptor (GlyRα2) (GenBank Accession No. NP_001112357.1, SEQ ID NO: 27), encoded by the GLRA2 gene (GenBank Accession No. NM_001118885.1):

```
(SEQ ID NO: 27)
                10 20 30 40 50 60
MNRQLVNILT ALFAFFLETN HERTAFCKDH DSRSGKQPSQ TLSPSDFLDK LMGRTSGYDA
                70 80 90 100 110 120
RIRPNFKGPP VNVTCNIFIN SFGSVTETTM DYRVNIFLRQ QWNDSRLAYS EYPDDSLDLD
                130 140 150 160 170 180
PSMLDSIWKP DLFFANEKGA NFHDVTTDNK LLRISKNGKV LYSIRLTLTL SCPMDLKNFP
                190 200 210 220 230 240
MDVQTCTMQL ESFGYTMNDL IFEWLSDGPV QVAEGLTLPQ FILKEEKELG YCTKHYNTGK
                250 260 270 280 290 300
FTCIEVKFHL ERQMGYYLIQ MYIPSLLIVI LSWVSFWINM DAAPARVALG ITTVLTMTTQ
                310 320 330 340 350 360
SSGSRASLPK VSYVKAIDIW MAVCLLFVFA ALLEYAAVNE VSRQHKEFLR LRRRQKRQNK
                370 380 390 400 410 420
EEDVTRESRF NESGYGMGHC LQVKDGTAVK ATPANPLPQP PKDGDAIKKK FVDRAKRIDT
                430 440 450 452
ISRAAFPLAF LIFNIFYWIT YKIIRHEDVH KK.
```

In some embodiments, the wild-type LGIC receptor is a human alpha 3 glycine receptor (GlyRα3) isoform L (GenBank Accession No. NP_006520.2, SEQ ID NO: 28), encoded by the GLRAβ gene (GenBank Accession No. NM_006529.3):

```
(SEQ ID NO: 28)
                10 20 30 40 50 60
MAHVRHERTL VSGFYFWEAA LLLSLVATKE TDSARSRSAP MSPSDFLDKL MGRTSGYDAR
                70 80 90 100 110 120
IRPNFKGPPV NVTCNIFINS FGSIAETTMD YRVNIFLRQK WNDPRLAYSE YPDDSLDLDP
                130 140 150 160 170 180
SMLDSIWKPD LFFANEKGAN FHEVTTDNKL LRIFKNGNVL YSIRLTLTLS CPMDLKNFPM
                190 200 210 220 230 240
DVQTCIMQLE SFGYTMNDLI FEWQDEAPVQ VAEGLTLPQF LLKEEKDLRY CTKHYNTGKF
                250 260 270 280 290 300
TCIEVRFHLE RQMGYYLIQM YIPSLLIVIL SWVSEWINMD AAPARVALGI TTVLTMTTQS
                310 320 330 340 350 360
SGSRASLPKV SYVKAIDIWM AVCLLEVESA LLEYAAVNEV SRQHKELLRF RRKRKNKTEA
                370 380 390 400 410 420
FALEKFYRES DMDDEVRESR FSFTAYGMGP CLQAKDGMTP KGPNHPVQVM PKSPDEMRKV
                430 440 450 460 464
FIDRAKKIDT ISRACFPLAF LIFNIFYWVI YKILRHEDIH QQQD.
```

In some embodiments, the wild-type LGIC receptor is a human alpha 3 glycine receptor (GlyRα3) isoform K (GenBank Accession No. NP_001036008.1, SEQ ID NO: 29), encoded by the GLRAβ gene (GenBank Accession No. NM_001042543.3):

```
(SEQ ID NO: 29)
                10 20 30 40 50
MAHVRHERTL VSGFYFWEAA LLLSLVATKE TDSARSRSAP MSPSDELDKL
                60 70 80 90 100
MGRTSGYDAR IRPNFKGPPV NVTCNIFINS FGSIAETTMD YRVNIFLRQK
                110 120 130 140 150
WNDPRLAYSE YPDDSLDLDP SMLDSIWKPD LFFANEKGAN FHEVTTDNKL
                160 170 180 190 200
LRIFKNGNVL YSIRLTLTLS CPMDLKNFPM DVQTCIMQLE SFGYTMNDLI
                210 220 230 240 250
FEWQDEAPVQ VAEGLTLPQF LLKEEKDLRY CTKHYNTGKF TCIEVRFHLE
                260 270 280 290 300
RQMGYYLIQM YIPSLLIVIL SWVSFWINMD AAPARVALGI TTVLTMTTQS
                310 320 330 340 350
SGSRASLPKV SYVKAIDIWM AVCLLEVESA LLEYAAVNEV SRQHKELLRE
                360 370 380 390 400
RRKRKNKDDE VRESRESFTA YGMGPCLQAK DGMTPKGPNH PVQVMPKSPD
                410 420 430 440
EMRKVFIDRA KKIDTISRAC FPLAFLIFNI FYWVIYKILR HEDIHQQQD.
```

In some aspects, the engineered receptor is a chimeric LGIC receptor. In some embodiments, the chimeric receptor comprises a ligand binding domain sequence derived from at least a first LGIC and an ion pore conduction domain sequence, or more simply, “ion pore domain sequence” derived from at least a second LGIC. In some embodiments, the derived amino acid sequence is identical to the corresponding region of the LGIC from which it was derived. In some embodiments, the derived amino acid sequence may contain alterations in at least one amino acid position compared to the corresponding region of the LGIC from which it was derived. In some embodiments, an amino acid sequence derived from the LGIC sequence differs by up to 1, 2, 3, 4, 5, 6, 7, 8, 9, or 10 amino acid residues from the corresponding region of the original amino acid sequence. In some embodiments, a derived amino acid sequence has at least 85%, at least 90%, at least 95%, at least 96%, at least 97%, at least 98%, at least 99%, or at least 99.5% (including all ranges and subranges therebetween) sequence identity to the corresponding region of the LGIC amino acid sequence.

In some embodiments, the first and second LGIC are Cys-loop receptors. Ligand binding domain sequences and ion pore domain sequences of the Cys-loop receptors are well known in the art and can be readily identified from the literature by use of publicly available software, e.g., PubMed, Genbank, Uniprot, and the like. In some embodiments, the ligand binding domain of the chimeric receptor has at least 85%, at least 90%, at least 95%, at least 96%, at least 97%, at least 98%, at least 99%, at least 99.5%, or 100% sequence identity to the ligand binding domain of the first LGIC. In some embodiments, the ion pore domain of the chimeric receptor has at least 85%, at least 90%, at least 95%, at least 96%, at least 97%, at least 98%, at least 99%, at least 99.5%, or 100% sequence identity to the ion pore domain of the second LGIC. In the sequences described above, the ligand binding domain is bolded, and the ion pore domain is underlined.

In some embodiments, the ligand binding domain of the chimeric receptor is derived from the ligand binding domain sequence of a human glycine receptor. In some embodiments, the human glycine receptor is human GlyRα1 (SEQ ID NO: 26). In some embodiments, the ligand binding domain comprises an amino acid sequence having at least 85%, at least 90%, at least 95%, at least 96%, at least 97%, at least 98%, at least 99%, at least 99.5%, or 100% sequence identity to about amino acids 29-235 of GlyRα1, e.g., amino acids 29-235, amino acids 29-240, amino acids 29-246, amino acids 29-248, amino acids 29-250, or amino acids 29-252 of SEQ ID NO: 26. In certain such embodiments, the ligand binding domain consists essentially of amino acids 29-235 of SEQ ID NO: 26, consists essentially of amino acids 29-240 of SEQ ID NO: 26, consists essentially of amino acids 29-246 of SEQ ID NO: 26, consists essentially of amino acids 29-248 of SEQ ID NO: 26, consists essentially of amino acids 29-250 of SEQ ID NO: 26, consists essentially of amino acids 29-252 of SEQ ID NO: 26. In some embodiments, the ion pore domain sequence is derived from a Cys-loop receptor other than the human GlyRα1.

In some embodiments, the ligand binding domain of the chimeric receptor comprises the ligand binding domain sequence of a human nicotinic cholinergic receptor. In some embodiments, the human nicotinic cholinergic receptor is human α7-nAChR. In some embodiments, the ligand binding domain comprises about amino acids 23-220 of human α7-nAChR (SEQ ID NO: 25), e.g., amino acids 23-220, amino acids 23-221, amino acids 23-222, amino acids 23-223, amino acids 23-224, amino acids 23-225, amino acids 23-226, amino acids 23-227, amino acids 23-228, amino acids 23-229, amino acids 23-230, or amino acids 23-231 of SEQ ID NO: 25. In some embodiments, the ligand binding domain consists essentially of amino acids 23-220, amino acids 23-221, amino acids 23-222, amino acids 23-223, amino acids 23-224, amino acids 23-225, amino acids 23-226, amino acids 23-227, amino acids 23-228, amino acids 23-229, amino acids 23-230, or amino acids 23-231 of SEQ ID NO: 25. In some embodiments, the ion pore domain sequence is derived from a Cys-loop receptor other than the human α7-nAChR.

In some embodiments, the ligand binding domain of the chimeric receptor is derived from the ligand binding domain sequence of a human nicotinic cholinergic receptor. In some embodiments, the human nicotinic cholinergic receptor is human α7-nAChR. In some embodiments, the ligand binding domain comprises an amino acid sequence having at least 85%, at least 90%, at least 95%, at least 96%, at least 97%, at least 98%, at least 99%, at least 99.5%, or 100% sequence identity to about amino acids 23-220 of human α7-nAChR (SEQ ID NO: 25), e.g., amino acids 23-220, amino acids 23-221, amino acids 23-222, amino acids 23-223, amino acids 23-224, amino acids 23-225, amino acids 23-226, amino acids 23-227, amino acids 23-228, amino acids 23-229, amino acids 23-230, or amino acids 23-231 of SEQ ID NO: 25. In some embodiments, the ion pore domain sequence is derived from a Cys-loop receptor other than the human α7-nAChR.

In some embodiments, the ion pore domain of the engineered receptor is derived from the ion pore domain sequence of a human glycine receptor. In some embodiments, the human glycine receptor is human GlyRα1. In some embodiments, the ion pore domain comprises an amino acid sequence having at least 85%, at least 90%, at least 95%, at least 96%, at least 97%, at least 98%, at least 99%, at least 99.5%, or 100% sequence identity to about amino acids 245-457 of GlyRα1 (SEQ ID NO: 26), e.g., amino acids 240-457, amino acids 245-457, amino acids 248-457, amino acids 249-457, amino acids 250-457, amino acids 255-457, or amino acids 260-457 of SEQ ID NO: 26. In some embodiments, the ion pore domain consists essentially of amino acids 245-457 of SEQ ID NO: 26, consists essentially of amino acids 248-457 of SEQ ID NO: 26, consists essentially of amino acids 249-457 of SEQ ID NO: 26, or consists essentially of amino acids 250-457 of SEQ ID NO: 26.

In some embodiments, the ion pore domain of the chimeric receptor comprises the ion pore domain sequence of human GlyRα2 (SEQ ID NO: 27). In some embodiments, the ion pore domain of the chimeric receptor comprises, consists essentially of, or consists of an amino acid sequence derived from the ion pore domain sequence of human GlyRα2 (SEQ ID NO: 27). In some embodiments, the ion pore domain of the chimeric receptor comprises, consists essentially of, or consists of an amino acid sequence having at least 85%, at least 90%, at least 95%, at least 96%, at least 97%, at least 98%, at least 99%, or at least 99.5% sequence identity to the ion pore domain sequence of human GlyRα2 (SEQ ID NO: 27). In some embodiments, the ion pore domain of the chimeric receptor comprises, consists essentially of, or consists of an amino acid sequence identical to the ion pore domain sequence of human GlyRα2 (SEQ ID NO: 27). In some embodiments, the ion pore domain sequence of human GlyRα2 comprises, consists essentially of, or consists of amino acids 254-452 of SEQ ID NO: 27. In some embodiments, the ion pore domain sequence of human GlyRα2 comprises, consists essentially of, or consists of amino acids 254-452 of SEQ ID NO: 27. In some embodiments, the ion pore domain sequence of human GlyRα2 comprises, consists essentially of, or consists of amino acids 258-452 of SEQ ID NO: 27. In some embodiments, the ion pore domain sequence of human GlyRα2 comprises, consists essentially of, or consists of amino acids 260-452 of SEQ ID NO: 27.

In some embodiments, the ion pore domain of the chimeric receptor comprises the ion pore domain sequence of human GlyRα3 isoform L (SEQ ID NO: 28). In some embodiments, the ion pore domain of the chimeric receptor comprises, consists essentially of, or consists of an amino acid sequence derived from the ion pore domain sequence of human GlyRα3 isoform L (SEQ ID NO: 28). In some embodiments, the ion pore domain of the chimeric receptor comprises, consists essentially of, or consists of an amino acid sequence having at least 85%, at least 90%, at least 95%, at least 96%, at least 97%, at least 98%, at least 99%, or at least 99.5% sequence identity to the ion pore domain sequence of human GlyRα3 isoform L (SEQ ID NO: 28). In some embodiments, the ion pore domain of the chimeric receptor comprises, consists essentially of, or consists of an amino acid sequence identical to the ion pore domain sequence of human GlyRα3 isoform L (SEQ ID NO: 28). In some embodiments, the ion pore domain sequence of human GlyRα3 isoform L comprises, consists essentially of, or consists of amino acids 253-464 of SEQ ID NO: 28. In some embodiments, the ion pore domain sequence of human GlyRα3 isoform L comprises, consists essentially of, or consists of amino acids 257-464 of SEQ ID NO: 28. In some embodiments, the ion pore domain sequence of human GlyRα3 isoform L comprises, consists essentially of, or consists of amino acids 259-464 of SEQ ID NO: 28.

In some embodiments, the ion pore domain of the chimeric receptor comprises the ion pore domain sequence of human GlyRα3 isoform K (SEQ ID NO: 29). In some embodiments, the ion pore domain of the chimeric receptor comprises, consists essentially of, or consists of an amino acid sequence derived from the ion pore domain sequence of human GlyRα3 isoform K (SEQ ID NO: 29). In some embodiments, the ion pore domain of the chimeric receptor comprises, consists essentially of, or consists of an amino acid sequence having at least 85%, at least 90%, at least 95%, at least 96%, at least 97%, at least 98%, at least 99%, or at least 99.5% sequence identity to the ion pore domain sequence of human GlyRα3 isoform K (SEQ ID NO: 29). In some embodiments, the ion pore domain of the chimeric receptor comprises, consists essentially of, or consists of an amino acid sequence identical to the ion pore domain sequence of human GlyRα3 isoform K (SEQ ID NO: 29). In some embodiments, the ion pore domain sequence of human GlyRα3 isoform K comprises, consists essentially of, or consists of amino acids 253-449 of SEQ ID NO: 29. In some embodiments, the ion pore domain sequence of human GlyRα3 isoform K comprises, consists essentially of, or consists of amino acids 257-449 of SEQ ID NO: 29. In some embodiments, the ion pore domain sequence of human GlyRα3 isoform K comprises, consists essentially of, or consists of amino acids 259-449 of SEQ ID NO: 29.

In some embodiments, the ion pore domain is derived from the ion pore domain sequence of a human nicotinic cholinergic receptor. In some embodiments, the human nicotinic cholinergic receptor is human α7-nAChR. In some embodiments, the ion pore domain comprises an amino acid sequence having at least 85%, at least 90%, at least 95%, at least 96%, at least 97%, at least 98%, at least 99%, at least 99.5%, or 100% sequence identity to about amino acids 230-502 of α7-nAChR (SEQ ID NO: 25), e.g., amino acids 227-502, amino acids 230-502, amino acids 231-502, amino acids 232-502, or amino acids 235-502. In certain such embodiments, the ion pore domain consists essentially of amino acids 227-502 of SEQ ID NO: 25, consists essentially of amino acids 230-502 of SEQ ID NO: 25, consists essentially of amino acids 231-502 of SEQ ID NO: 25, consists essentially of amino acids 232-502 of SEQ ID NO: 25, or consists essentially of amino acids 235-502 of SEQ ID NO: 25.

In some embodiments, the ion pore domain of the subject chimeric ligand-gated ion channel comprises an M2-M3 linker domain that is heterologous to the M2-M3 linker domain of the ion pore domain. By an “M2-M3 linker domain”, or “M2-M3 linker”, it is meant the sequence within an ion pore domain of a LGIC that is flanked at its amino (N) terminus by the C-terminal end of transmembrane domain 2 (M2) of the receptor and at its carboxy (C) terminus by the N-terminal end of transmembrane domain 3 (M3) of the receptor. The M2-M3 linker of a LGIC may be readily determined from the art and/or by using any publicly available protein analysis tool, e.g., Expasy, uniProt, etc. In some embodiments, when the ion pore domain of a chimeric receptor comprises a heterologous M2-M3 linker, the M2-M3 linker is derived from the same receptor as the ligand binding domain of the chimeric receptor. For example, when the subject ligand-gated ion channel comprises a ligand binding domain from an AChR and an ion pore domain from a GlyR, its ion pore domain sequence may comprise a M2-M3 linker sequence derived from the AChR. In some embodiments, the ion pore domain is derived from GlyRα1 and the M2-M3 linker is derived from α7-nAChR. In some embodiments, the native M2-M3 linker sequence that is removed from the ion pore domain corresponds to about amino acids 293-313, of GlyRα1 (SEQ ID NO: 26), e.g., amino acids 304-310, 293-306, 298-310, 305-311, 302-313, etc. In some such embodiments, the M2-M3 linker that is inserted is derived from about amino acids 281-295 of α7-nAChR (SEQ ID NO: 25), e.g., amino acids 290-295, 281-290, 281-295, 283-295, 287-292, etc. or a sequence at least 50%, at least 55%, at least 60%, at least 65%, at least 70%, at least 75%, at least 80%, at least 85%, at least 90%, at least 95%, or 100% identical to amino acids 281-295 or 283-295 of α7-nAChR (SEQ ID NO: 25).

In some embodiments, the ligand binding domain of the subject chimeric ligand-gated ion channel comprises a Cys-loop domain sequence that is heterologous to the Cys-loop sequence of the ligand binding domain. By a “Cys-loop domain sequence”, or “Cys-loop sequence”, it is meant the domain within a ligand binding domain of a Cys-loop LGIC that forms a loop structure flanked by a cysteine at the N-terminus and the C-terminus. Without wishing to be bound by theory, it is believed that upon binding of the ligand to the ligand binding domain, the Cys-loop structurally moves to be in close proximity to the M2-M3 loop, this movement mediating the biophysical translation of ligand binding in the extracellular domain to signal transduction in the ion pore domain (as reviewed in Miller and Smart, Trends in Pharmacological Sci 2009:31(4)). The substitution of an endogenous Cys-loop sequence with a heterologous Cys-loop sequence may increase the conductivity of the LGIC by 1.5-fold or more, e.g., at least 2-fold, 3-fold or 4-fold, in some instances at least 5-fold or 6-fold, and at certain doses, at least 7-fold, 8-fold, 9-fold or 10-fold. The Cys-loop domain of a Cys-loop receptor may be readily determined from the art and/or by using any publicly available protein analysis tool, e.g., Expasy, uniProt, etc. Typically, when the ligand binding domain of a chimeric receptor comprises a heterologous Cys-loop sequence, the Cys-loop sequence is derived from the same receptor as the ion pore domain of the chimeric receptor. For example, when the subject chimeric ligand-gated ion channel comprises a ligand binding domain from an AChR and an ion pore domain from a GlyR, the subject ligand-gated ion channel may comprise ligand binding domain sequence from an AChR except for the sequence of the Cys-loop domain, which is instead derived from a GlyR. In some embodiments, the ligand binding domain is derived from α7-nAChR and the Cys-loop sequence is derived from a GLyR. In some embodiments, the Cys-loop sequence that is removed from the ligand binding domain corresponds to about amino acids 150-164 of α7-nAChR (SEQ ID NO: 25), e.g., amino acids 150-157 of α7-nAChR. In some embodiments, the Cys loop sequence that is inserted is derived from about amino acids 166-180 of GlyRα1 (SEQ ID NO: 26), e.g., amino acids 166-172 of GlyRα1, or a sequence at least 80%, at least 85%, at least 90%, or at least 95% identical to amino acids 166-180 of GlyRα1. In some embodiments, the Cys loop sequence that is inserted is derived from about amino acids 172-186 of GlyRα2 (SEQ ID NO: 27), e.g., amino acids 172-178 of GlyRα2, or a sequence at least 80%, at least 85%, at least 90%, or at least 95% identical to amino acids 172-186 of GlyRα2. In some embodiments, the Cys loop sequence that is inserted is derived from about amino acids 171-185 of GlyRα3 (SEQ ID NO: 28 or 29), e.g., amino acids 171-177 of GlyRα3, or a sequence at least 80%, at least 85%, at least 90%, or at least 95% identical to amino acids 171-185 of GlyRα3.

In some embodiments, the ligand binding domain of the subject chimeric ligand-gated ion channel comprises a β1-2 loop domain sequence that is heterologous to the β1-2 loop domain sequence of the ligand binding domain. By a “β1-2 loop domain sequence”, or “β1-2 loop, or β1-β2 loop”, it is meant the domain within a ligand binding domain of a Cys-loop LGIC that is flanked at its N-terminus by the C-terminus of the β1 sheet and, at its C-terminus, by the N-terminus of the β2 sheet. Without wishing to be bound by theory, it is believed that the β1-2 loop helps to mediate biophysical translation of ligand binding in the extracellular domain to the ion pore domain and subsequent signal transduction (i.e. chloride influx in case of GlyR). It is believed that upon binding of ligand, the β1-2 loop, together with the Cys-loop, come in close proximity to the M2-M3 loop to mediate the biophysical translation of ligand binding in the extracellular domain to signal transduction in the ion pore domain where the M2-M3 loop resides (as reviewed in Miller and Smart, supra). The substitution of an endogenous β1-2 loop sequence with a heterologous β1-2 loop sequence may increase the conductivity of the LGIC by 1.5-fold or more, e.g., at least 2-fold, 3-fold or 4-fold, in some instances at least 5-fold or 6-fold, and at certain doses, at least 7-fold, 8-fold, 9-fold or 10-fold. The β1-2 loop of a Cys-loop receptor may be readily determined from the art and/or by using any publicly available protein analysis tool, e.g., Expasy, uniProt, etc. Typically, when the ligand binding domain of a chimeric receptor comprises a heterologous β1-2 loop sequence, the β1-2 loop sequence is derived from the same receptor as the ion pore domain of the chimeric receptor. For example, when the subject chimeric ligand-gated ion channel comprises a ligand binding domain derived from an AChR and an ion pore domain derived from a GlyR, the sequence of the β1-2 loop domain of the ligand binding domain may be derived from a GlyR. In some embodiments, the ligand binding domain is derived from α7-nAChR. In some embodiments, the β1-2 loop sequence that is removed from the ligand binding domain corresponds to about amino acids 64-72 or 67-70 of α7-nAChR (SEQ ID NO: 25), e.g., amino acids 67-70, 66-71 or 64-72 of α7-nAChR. In some embodiments, the β1-2 loop sequence that is inserted is about amino acids 79-85 of GlyRα1 (SEQ ID NO: 26), e.g., amino acids 80-85, 81-84, 79-85, or 81-84 of GlyRα1, with at most 3, at most 2, at most 1, or no amino acid mutations. In some embodiments, the ion pore domain is derived from GlyRα2 and the β1-2 loop that is inserted corresponds to about amino acids 86-91 of GlyRα2 (SEQ ID NO: 27) with at most 3, at most 2, at most 1, or no amino acid mutations. In some embodiments, the ion pore domain is derived from GlyRα3 and the β1-2 loop that is inserted corresponds to about amino acids 85-90 of GlyRα3 (SEQ ID NO: 28 or 29) with at most 3, at most 2, at most 1, or no amino acid mutations. In some embodiments, the mutation is an amino acid substitution.

In some embodiments, the disclosure provides chimeric LGIC receptors comprising a ligand binding domain derived from human α7-nAChR, wherein the ligand binding domain comprises one or more amino acid substitutions of the disclosure, and an ion pore domain derived from a human Glycine receptor. In some embodiments, the human Glycine receptor is human Glycine receptor α1, human Glycine receptor α2, or human Glycine receptor α3. In some embodiments, the ligand binding domain comprises a Cys-loop domain derived from the human Glycine receptor. In some embodiments, the ligand binding domain comprises a β1-2 loop domain derived from the human Glycine receptor.

Non-limiting examples of sequences of chimeric LGIC receptors of the present disclosure include the sequences disclosed herein as SEQ ID NO: 30-31 and 33. In some embodiments, the chimeric LGIC receptor has a sequence identity of 85% or more to a sequence provided in SEQ ID NO: 30-31 and 33 herein, e.g., a sequence identity of 90% or more, 93% or more, 95% or more, 96% or more, 97% or more, 98% or more, 99% or more, or 100% to a sequence provided in SEQ ID NO: 30-31 and 33. In the sequences, the signal peptide is italicized, the ligand binding domain is bolded, and the ion pore domain is underlined.

In some embodiments, the chimeric LGIC receptor is a CHRNA7/GLRA1 chimera comprising the human α7-nAChR signal peptide (italics) and ligand binding domain (bold) comprising an GlyRα1 Cys-loop sequence (lowercase); fused to the human GlyRα1 ion pore domain (underlined). In some embodiments, the chimeric LGIC receptor comprises an amino acid sequence having a sequence identity of 80% or more, 85% or more, 90% or more, 95% or more, 96% or more, 97% or more, 98% or more, 99% or more, or 100%, to SEQ ID NO: 33:

```
(SEQ ID NO: 33, encoded by SEQ ID NO: 32)
                10 20 30 40 50 60
MRCSPGGVWL ALAASLLHVS LQGEFQRKLY KELVKNYNPL ERPVANDSQP LTVYFSLSLL
                70 80 90 100 110 120
QIMDVDEKNQ VLTTNIWLQM SWTDHYLQWN VSEYPGVKTV RFPDGQIWKP DILLYNSADE
                130 140 150 160 170 180
RFDATFHTNV LVNSSGHCQY LPPGIFKSSc pmdlknfpmd vqtcKLKFGS WSYGGWSLDL
                190 200 210 220 230 240
QMQEADISGY IPNGEWDLVG IPGKRSERFY ECCKEPYPDV TFTVTMRRRM GYYLIQMYIP
                250 260 270 280 290 300
SLLIVILSWI SEWINMDAAP ARVGLGITTV LTMTTQSSGS RASLPKVSYV KAIDIWMAVC
                310 320 330 340 350 360
LLEVESALLE YAAVNFVSRQ HKELLRERRK RRHHKSPMLN LFQEDEAGEG RENESAYGMG
                370 380 390 400 410 420
PACLQAKDGI SVKGANNSNT TNPPPAPSKS PEEMRKLFIQ RAKKIDKISR IGFPMAFLIF
                430 439
NMFYWIIYKI VRREDVHNQ.
```

In some embodiments, the chimeric LGIC receptor is a CHRNA7/GLRA1 chimera comprising the human α7-nAChR signal peptide (italics) and ligand binding domain (bold) comprising an GlyRα1 β1-2 loop sequence (lowercase) and Cys-loop sequence (lowercase); fused to the human GlyRα1 ion pore domain (underlined):

MRCSPGGVWLALAASLLHVSLQGEFQRKLYKELVKNYNPLERPVANDSQ
PLTVYFSLSLLQIMDVDettmVLTTNIWLQMSWTDHYLQWNVSEYPGVK
TVRFPDGQIWKPDILLYNSADERFDATFHTNVLVNSSGHCQYLPPGIFK
SScpmdlknfpmdvqtcKLKFGSWSYGGWSLDLQMQEADISGYIPNGEW
DLVGIPGKRSERFYECCKEPYPDVTFTVTMRRRMGYYLIQMYIPSLLIV
ILSWISFWINMDAAPARVGLGITTVLTMTTQSSGSRASLPKVSYVKAID
IWMAVCLLFVFSALLEYAAVNFVSRQHKELLRFRRKRRHHKSPMLNLFQ
EDEAGEGRFNFSAYGMGPACLQAKDGISVKGANNSNTTNPPPAPSKSPE
EMRKLFIQRAKKIDKISRIGFPMAFLIFNMFYWIIYKIVRREDVHNQ

### (SEQ ID NO: 30).

In some embodiments, the chimeric LGIC receptor is a CHRNA7/GLRA1 chimera (R229 junction), comprising the human α7-nAChR signal peptide (italics) and ligand binding domain (bold), fused to the human GlyRα1 ion pore domain (underlined):

(SEQ ID NO: 31)
MRCSPGGVWLALAASLLHVSLQGEFQRKLYKELVKNYNPLERPVANDSQ
PLTVYFSLSLLQIMDVDEKNQVLTTNIWLQMSWTDHYLQWNVSEYPGVK
TVRFPDGQIWKPDILLYNSADERFDATFHTNVLVNSSGHCQYLPPGIFK
SSCYIDVRWFPFDVQHCKLKFGSWSYGGWSLDLQMQEADISGYIPNGEW
DLVGIPGKRSERFYECCKEPYPDVTFTVTMRRRMGYYLIQMYIPSLLIV
ILSWISFWINMDAAPARVGLGITTVLTMTTQSSGSRASLPKVSYVKAID
IWMAVCLLFVFSALLEYAAVNEVSRQHKELLRFRRKRRHHKSPMLNLFQ
EDEAGEGRFNFSAYGMGPACLQAKDGISVKGANNSNTTNPPPAPSKSPE
EMRKLFIQRAKKIDKISRIGFPMAFLIENMFYWIIYKIVRREDVHNQ

### Amino Acid Mutations in the Ligand-Gated Ion Channel

As discussed above, in some aspects, the subject engineered receptor comprises at least one amino acid mutation that alters the potency of a ligand on the engineered receptor relative to its potency on the unmutated parental receptor. Put another way, the one or more amino acid mutations, e.g., a loss-of-function mutations or a gain-of-function mutations, shift the potency of the engineered receptor to the ligand relative to the potency of the unmutated parental receptor. In some embodiments, the mutation is an amino acid substitution. In some embodiments, the one or more mutations is in the ligand binding domain of the engineered receptor. In some embodiments, as when the ligand binding domain of the engineered receptor is a Cys-loop receptor protein, the one or more amino acid mutations is a substitution at a residue corresponding to a residue of α7-nAChR (SEQ ID NO: 25) selected from the group consisting of W77, Y94, R101, W108, Y115, T128, N129, V130, L131, Q139, L141, Y151, S170, W171, S172, S188, Y190, Y210, C212, C213 and Y217. In some embodiments, one residue is substituted. In some embodiments, 2, 3, 4, or 5 or more residues are substituted, e.g., 6, 7, 8, 9 or 10 residues are substituted. In certain embodiments, the residue corresponds to a residue of α7-nAChR (SEQ ID NO: 25) that is selected from the group consisting of W77, R101, Y115, N129, L131, S170, S172, and S188. In certain embodiments, the one or more substitutions is within an α7-nAChR sequence.

In some embodiments, the one or more substitutions decreases, e.g., 2-fold or more, 3-fold or more, 4-fold or more. 5-fold or more, 10-fold or more, 20-fold or more, 30-fold or more, 50-fold or more, or 100-fold, the potency of an engineered receptor to acetylcholine and a non-native ligand. In certain embodiments, the one or more substitutions is a substitution corresponding to R101I, R101S, R101D, Y115L, Y115M, Y115D, Y115T, T128M, T128R, T128I, N129I, N129V, N129P, N129W, N129T, N129D, N129E, L131E, L131P, L131T, L131D, L131S, L141S, L141R, W171F, W171H, S172F, S172Y, S172R, S172D, C212A, C212L, or C213P of α7-nAChR. In other instances, the one or more substitutions decreases the potency of acetylcholine on the engineered receptor selectively. In other words, the one or more substitutions decreases the potency of the engineered receptor to acetylcholine while essentially maintaining potency to non-native ligand or otherwise decreasing the potency of the engineered receptor to acetylcholine 2-fold or more, e.g., 3-fold, 4-fold, 5-fold or more, in some instances 10-fold, 20-fold, 50-fold, or 100-fold or more, than it decreases the potency of the engineered receptor to non-native ligand. In some embodiments, the substitution corresponds to L131E, L131S, L131T, L131D, or S172D of α7-nAChR. In yet other embodiments, the one or more substitutions decreases the potency of a non-native ligand on the engineered receptor selectively. In other words, the one or more substitutions decreases the potency of the engineered receptor to non-native ligand while essentially maintaining potency to acetylcholine or otherwise decreasing the potency of the engineered receptor to non-native ligand 2-fold or more, e.g., 3-fold, 5-fold or more, in some instances 10-fold, 20-fold or 50-fold or more, than it decreases the potency of the engineered receptor to acetylcholine. In some embodiments, the substitution corresponds to W77M, Y115W, S172T, or S172C of α7-nAChR. In certain embodiments, the one or more substitutions is within an α7-nAChR sequence. In certain embodiments, the non-native ligand is selected from AZD-0328, TC6987, ABT-126, and Facinicline/RG3487.

In other embodiments, the one or more substitutions increases, e.g., 2-fold or more, 3-fold or more, 4-fold or more. 5-fold or more, 10-fold or more, 20-fold or more, 30-fold or more, 50-fold or more, or 100-fold, the potency of the engineered receptor to acetylcholine and/or non-native ligand. In some embodiments, the substitution corresponds to L131N, L141W, S170G, S170A, S170L, S170I, S170V, S170P, S170F, S170M, S170T, S170C, S172T, S172C, S188I, S188V, S188F, S188M, S188Q, S188T, S188P or S188W. In some embodiments, the one or more substitutions increases potency of both acetylcholine and non-native ligand. In some embodiments, the substitution corresponds to L131N, S170G, S170A, S170L, S170I, S170V, S170P, S170F, S170M, S170T, S170C, S172T, S188I, S188V, S188F, S188M, S188Q or S188T of α7-nAChR. In other instances, the one or more substitutions increases the potency of acetylcholine on the engineered receptor selectively. In other words, the one or more substitutions increases the potency of the engineered receptor to acetylcholine 2-fold or more, e.g., 3-fold, 4-fold, or 5-fold or more, in some instances 10-fold, 20-fold, 50-fold, or 100-fold, than it increases the potency of the engineered receptor to non-native ligand. In some embodiments, the substitution corresponds to L141W, S172T, S172C, S188P or S188W, of α7-nAChR. In certain embodiments, the one or more substitutions is within an α7-nAChR sequence. In certain embodiments, the non-native ligand is selected from AZD-0328, TC-5619, TC6987, ABT-126 and Facinicline/RG3487. In yet other instances, the one or more substitutions increases the potency of the non-native ligand on the engineered receptor selectively. In other words, the one or more substitutions increases the potency of the engineered receptor to non-native ligand 2-fold or more, e.g., 3-fold, 5-fold or more, in some instances 10-fold, 20-fold or 50-fold or more, than it increases the potency of the engineered receptor to acetylcholine.

In some embodiments, the amino acid residue that is mutated in the subject engineered receptor is not an amino acid corresponding to R27, E41, Q79, Q139, L141, G175, Y210, P216, Y217, or D219 of wild type α7 nAChR (SEQ ID NO: 25). In some embodiments, the mutation is an amino acid substitution. In some embodiments, the amino acid residue that is mutated in the subject engineered receptor is an amino acid corresponding to R27, E41, Q79, Q139, L141, G175, Y210, P216, Y217, or D219 of wild type α7 nAChR (SEQ ID NO: 25). In some embodiments, the substitution is not a substitution corresponding to W77F, W77Y, W77M, Q79A, Q79Q, Q79S, Q79G, Y115F, L131A, L131G, L131M, L131N, L131Q, L131V, L131F, Q139G, Q139L, G175K, G175A, G175F, G175H, G175M, G175R, G175S, G175V, Y210F, P216I, Y217F, or D219A in wild type α7 nAChR. In some embodiments, the substitution is a substitution corresponding to W77F, W77Y, W77M, Q79A, Q79Q, Q79S, Q79G, Y115F, L131A, L131G, L131M, L131N, L131Q, L131V, L131F, Q139G, Q139L, G175K, G175A, G175F, G175H, G175M, G175R, G175S, G175V, Y210F, P216I, Y217F, or D219A in wild type α7 nAChR. In some embodiments, when such a substitution exists within the engineered receptor, it exists in combination with one or more of the amino acid mutations described herein.

In some embodiments, residues Y94, Y115, Y151, and Y190 of α7-nAChR (SEQ ID NO: 25) mediate binding of the native ligand acetylcholine. In some embodiments, mutations at these residues may reduce binding of acetylcholine and hence be considered loss of function mutations. In some embodiments, residues W77, Y115, N129, V130, L131, Q139, L141, S170, Y210, C212, C213 and Y217 of the α7-nAChR may mediate the binding of non-native ligand AZD0328 to this receptor, and mutation of these residues may increase the affinity of AZD0328 and/or other ligands for this receptor and hence be considered gain-of-function mutations. In some embodiments, the subject engineered receptor comprises a mutation in one or more amino acid residues of the ligand binding domain region of α7-nAChR (SEQ ID NO: 25) or the ligand binding domain of a chimeric receptor that comprises the ligand binding domain region of α7-nAChR, wherein the one or more amino acid residues is selected from the group consisting of W77, Y94, Y115, N129, V130, L131, Q139, L141, Y151, S170, Y190, Y210, C212, C213 and Y217. In some embodiments, the mutation is an amino acid substitution. In certain embodiments, the mutation in the one or more amino acid residues of the ligand binding domain region of α7-nAChR (SEQ ID NO: 25) or the ligand binding domain of a chimeric receptor that comprises the ligand binding domain region of α7-nAChR is a substitution at one or more amino acid residues selected from the group consisting of W77, Y94, Y115, N129, V130, L131, Q139, L141, Y151, S170, Y190, Y210, C212, C213 and Y217.

In some embodiments, residues Y115, L131, L141, S170, W171, S172, C212, and Y217 of α7-nAChR (SEQ ID NO: 25) may mediate binding of acetylcholine and/or nicotine, and mutations at one or more of these residues may reduce binding of acetylcholine and/or nicotine. In some embodiments, R101, Y115, L131, L141, W171, S172, S188, Y210, and Y217 of α7-nAChR may mediate binding of the non-native ligand ABT126, and mutation of one or more of these residues may increase the affinity of ABT126 and/or other ligands for α7-nAChR. In some embodiments, the mutation is an amino acid substitution. In some embodiments, R101, Y115, T128, N129, L131, L141, W171, S172, Y210, C212, C213 and Y217 of α7-nAChR may mediate binding of the non-native ligand TC6987, and mutation of one or more of these residues may increase the affinity of TC6987 and/or other ligands for α7-nAChR. In some embodiments, R101, N120, L131, L141, S170, W171, S172, Y210, and Y217 of α7-nAChR may mediate binding of the non-native ligand Facinicline/RG3487, and mutation of one or more of these residues may increase the affinity of Facinicline/RG3487 and/or other ligands for α7-nAChR. In some embodiments, the subject engineered receptor comprises a mutation in one or more amino acid residues of the ligand binding domain region of α7-nAChR or the ligand binding domain of a chimeric receptor that comprises the ligand binding domain region of α7-nAChR, where the one or more amino acid residues is selected from the group consisting of R101, Y115, T128, N120, N129, L131, L141, S170, W171, S172, S188, Y210, C212, C213 and Y217. In some embodiments, the one or more amino acid residues alters the binding of acetylcholine and/or nicotine to α7-nAChR, wherein the amino acid is selected from the group consisting of Y115, L131, L141, S170, W171, S172, C212 and Y217 of α7-nAChR. In certain such embodiments, the amino acid is selected from C212 and S170. In some embodiments, the mutation in the one or more amino acid residues alters the binding of ABT126 to α7-nAChR, wherein one or more amino acid residues is selected from the group consisting of R101, Y115, L131, L141, W171, S172, S188, Y210, and Y217 of α7-nAChR. In certain such embodiments, the amino acid is selected from R101, S188, and Y210. In some embodiments, the mutation in the one or more amino acid residues alters the binding of TC6987 to α7-nAChR, wherein one or more amino acid residues is selected from the group consisting of R101, Y115, T128, N129, L131, L141, W171, S172, Y210, C212, C213 and Y217 of α7-nAChR. In certain such embodiments, the amino acid is selected from R101, T128, N129, Y210 and C213. In some embodiments, the mutation in the one or more amino acid residues alters the binding of Facinicline/RG3487 to α7-nAChR, wherein one or more amino acid residues is selected from the group consisting R101, N120, L131, L141, S170, W171, S172, Y210, and Y217 of α7-nAChR. In certain such embodiments, the amino acid is selected from Y210, R101, and N129.

The disclosure provides engineered receptors having two or more mutations, such as amino acid substitutions, as compared to the parental receptor. In some embodiments, the parental receptor comprises a ligand binding domain derived from human α7 nicotinic acetylcholine receptor (α7-nAChR). In some embodiments, the parental receptor is a chimeric receptor. In some embodiments, the parental receptor comprises an ion pore domain derived from a human Glycine receptor. In some embodiments, the human Glycine receptor is human Glycine receptor α1, human Glycine receptor α2, or human Glycine receptor α3. In some embodiments, the ligand binding domain of the engineered receptor comprises a Cys-loop domain derived from the human Glycine receptor. In some embodiments, the parental receptor comprises an amino acid sequence of SEQ ID NO: 33. In some embodiments, the engineered receptors comprise two amino acid substitutions as compared to the parental receptor comprising an amino acid sequence of SEQ ID NO: 33. In some embodiments, the ligand binding domain of the engineered receptor comprises a β1-2 loop domain from the human Glycine receptor α1 subunit.

In some embodiments, the ligand binding domain of the engineered receptor comprises amino acid substitutions at two or more amino acid residues selected from those corresponding to W77, R101, Y115, L131, Q139, Y140, S170, S172 and Y210 of human α7-nAChR (SEQ ID NO: 25).

In some embodiments, the two amino acid substitutions are at a pair of amino acid residues selected from the group consisting of L131 and S172, Y115 and S170, and Y115 and L131. In some embodiments, the ligand binding domain comprises two amino acid substitutions at a pair of amino acids residues selected from the group consisting of L131 and S172, Y115 and S170, and Y115 and L131. In some embodiments, the ligand binding domain comprises an amino acid substitution at residue L131 and the amino acid substitution of S172D. In some embodiments, the ligand binding domain comprises an amino acid substitution at residue L131 and the amino acid substitution of Y115D. In some embodiments, the ligand binding domain comprises a pair of amino acid substitutions selected from the group consisting of L131S and S172D, L131T and S172D, L131D and S172D, Y115D and S170T, Y115D and L131Q, and Y115D and L131E. In some embodiments, the ligand binding domain comprises an amino acid substitution of L131E.

In some embodiments, the ligand binding domain comprises one or more amino acid substitutions at amino acids residues selected from the group consisting of Y140, R101, L131, Y115, and Y210, wherein the amino acid residues correspond to the amino acid residues of α7-nAChR. In some embodiments, the ligand binding domain comprises an amino acid substitution of R101W and/or Y210V. In some embodiments, the ligand binding domain comprises two or more amino acid substitutions at amino acid residues selected from the group consisting of R101, L131, Y115, Y210, and Y140. In some embodiments, the ligand binding domain comprises two amino acid substitutions at amino acid residues selected from the group consisting of R101, L131, Y115, Y210, and Y140. In some embodiments, the ligand binding domain comprises two amino acid substitutions at a pair of amino acid residues selected from the group consisting of: R101 and L131, Y115 and Y210, R101 and Y210. In some embodiments, the ligand binding domain comprises a pair of amino acid substitutions selected from the group consisting of R101F and L131G, R101F and L131D, Y115E and Y210W, R101W and Y210V, R101F and Y210V, R101F and Y210F, R101M and L131A, and R101M and L131F. In some embodiments, the ligand binding domain comprises three amino acid substitutions at the amino acid residues R101, Y115, and Y210. In some embodiments, the ligand binding domain comprises amino acid substitutions R101W, Y115E, and Y210W, or the amino acid substitutions R101F, Y115E, and Y210W.

In some embodiments, the ligand binding domain comprises an amino acid substitution at residue L131 and the amino acid substitution of R101F or R101M. In some embodiments, the amino acid substitution at residue L131 is L131G, L131D, L131A, L131F, or L131N.

In some embodiments, the ligand binding domain comprises a hydrophobic amino acid substitution at residue Y210 and the amino acid substitution of R101W or R101F. In some embodiments, the amino acid substitution at residue Y210 is Y210V, Y210F, or Y210W.

A person skilled in the art will readily recognize proper control receptor for comparison with the engineered receptor of the disclosure. In some embodiments, the control receptor is identical in sequence to the engineered receptor except for the one or more distinguishing amino acid mutations (e.g., substitutions). In all cases, references to a control receptor is meant to indicate that the recited change of property (e.g., potency to a ligand) is the result of the amino acid mutation(s) of the engineered receptor of the disclosure.

The disclosure provides engineered receptors, wherein the engineered receptor is a chimeric ligand gated ion channel (LGIC) receptor and comprises: (a) a ligand binding domain derived from the human α7 nicotinic acetylcholine receptor (α7-nAChR) and comprising a Cys-loop domain from the human Glycine receptor α1 subunit; and (b) an ion pore domain derived from the human Glycine receptor α1 subunit. In some embodiments, the engineered receptor is derived from a parental engineered receptor comprising or consisting of an amino acid sequence of SEQ ID NO: 33, and further comprises one or more amino acid substitutions based on the parental engineered receptor.

In some embodiments, the potency of the engineered receptor to acetylcholine is lower than the potency of the human α7 nicotinic acetylcholine receptor (α7-nAChR) to acetylcholine. In some embodiments, the potency of the engineered receptor to acetylcholine is at least 1.5-fold (for example, about 2-fold lower, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, about 60-fold, about 70-fold, about 80-fold, about 90-fold, or about 100-fold, including all subranges and values that lie therebetween) lower than the potency of the human α7 nicotinic acetylcholine receptor (α7-nAChR) to acetylcholine. In some embodiments, the potency of the engineered receptor to acetylcholine is evaluated by its EC50 for acetylcholine based on a cell reporter assay using YFP fluorescence quenching. In some embodiments, the EC50 of the engineered receptor to acetylcholine is at least 100 uM, at least 200 uM, at least 300 uM, at least 500 uM, at least 700 uM, at least 1 mM, at least 2 mM, at least 3 mM, at least 4 mM, at least 5 mM, at least 6 mM, at least 7 mM, at least 8 mM, at least 9 mM, or at least 10 mM. In some embodiments, the EC50 of the engineered receptor to acetylcholine is at least 1 mM. In some embodiments, the EC50 of the engineered receptor to acetylcholine is at least 3 mM. In some embodiments, having a higher EC50 for acetylcholine permits higher expression level of the engineered receptor in the cell or on the cell surface, without passing significant amount of current into the cell at the presence of physiological concentration of acetylcholine.

In some embodiments, the potency of the engineered receptor to a non-native ligand is about the same as the potency of the human α7 nicotinic acetylcholine receptor (α7-nAChR) to the non-native ligand. In some embodiments, the potency of the engineered receptor to a non-native ligand is higher than the potency of the human α7 nicotinic acetylcholine receptor (α7-nAChR) to the non-native ligand. In some embodiments, the potency of the engineered receptor to the non-native ligand is at least 1.5-fold (for example, about 2-fold lower, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, about 60-fold, about 70-fold, about 80-fold, about 90-fold, or about 100-fold, including all subranges and values that lie therebetween) higher than the potency of the human α7 nicotinic acetylcholine receptor (α7-nAChR) to the non-native ligand. In some embodiments, determining the potency comprises determining the EC50 based on a cell reporter assay using YFP fluorescence quenching as described in Example 2 of the disclosure. In some embodiments, the EC50 of the engineered receptor to a non-native ligand is less than 1 nM, less than 2 nM, less than 3 nM, less than 4 nM, less than 5 nM, less than 6 nM, less than 7 nM, less than 8 nM, less than 9 nM, less than 10 nM, less than 15 nM, less than 20 nM, less than 30 nM, less than 40 nM, less than 50 nM, less than 60 nM, less than 70 nM, less than 80 nM, less than 90 nM, less than 100 nM, less than 150 nM, less than 200 nM, less than 300 nM, less than 400 nM, less than 500 nM, less than 600 nM, less than 700 nM, less than 800 nM, less than 900 nM, less than 1 uM, less than 2 uM, less than 3 uM, less than 4 uM, less than 5 uM, less than 6 uM, less than 7 uM, less than 8 uM, less than 9 uM, or less than 10 uM. In some embodiments, the EC50 of the engineered receptor to a non-native ligand is less than 10 nM. In some embodiments, the EC50 of the engineered receptor to a non-native ligand is less than 100 nM. In some embodiments, the EC50 of the engineered receptor to a non-native ligand is less than 1 uM.

In some embodiments, the efficacy of the engineered receptor in the presence of a non-native ligand is higher than the efficacy the human α7 nicotinic acetylcholine receptor (α7-nAChR) in presence of the non-native ligand. In some embodiments, the efficacy of the engineered receptor in the presence of a non-native ligand is at least 1.5-fold (for example, about 2-fold lower, about 3-fold, about 4-fold, about 5-fold, about 6-fold, about 7-fold, about 8-fold, about 9-fold, about 10-fold, about 12-fold, about 15-fold, about 20-fold, about 30-fold, about 40-fold, about 50-fold, about 60-fold, about 70-fold, about 80-fold, about 90-fold, or about 100-fold, including all subranges and values that lie therebetween) higher than the efficacy the human α7 nicotinic acetylcholine receptor (α7-nAChR) in presence of the non-native ligand. In some embodiments, determining the efficacy comprises determining the amount of current passed through the engineered receptor in vitro in the presence of the non-native ligand.

In some aspects, the subject ligand-gated ion channel comprises one or more non-desensitizing mutations. In some embodiments, the mutation is an amino acid substitution. When used in the context of a ligand-gated ion channel, “desensitization” refers to the progressive reduction in ionic flux in the prolonged presence of agonist. This results in a progressive loss of potency of the neuron to the ligand. By a non-desensitizing mutation, it is meant an amino acid mutation that prevents the LGIC from becoming desensitized to ligand, thereby preventing the neuron from becoming less responsive or nonresponsive to ligand. Non-desensitizing mutations can be readily identified by introducing the LGIC carrying the mutation into a neuron and analyzing the current flux over time during prolonged exposure to ligand. If the LGIC does not comprise a non-desensitizing mutation, the current will restore from peak to steady state during prolonged exposure, whereas if the LGIC comprises a non-desensitizing mutation, the current will remain at peak flux for the duration of exposure to ligand. Exemplary amino acid mutations that result in desensitization include a V322L mutation in the human GlyRα1 (V294L post-processing of the pro-protein to remove the signal peptide). LGIC desensitization, methods for measuring desensitization of LGICs, and mutations that are non-desensitizing are well known in the art; see, e.g., Gielen et al. Nat Commun 2015 Apr. 20, 6:6829, and Keramidas et al. Cell Mol Life Sci. 2013 April; 70(7):1241-53, the full disclosures of which are incorporated herein by reference.

In some aspects, the subject ligand-gated ion channel comprises one or more conversion mutations. In some embodiments, the mutation is an amino acid substitution. By a conversion mutation, it is meant a mutation that changes the permeability of the ion pore domain of the LGIC such that it becomes permissive to the conductance of a non-native ion, i.e. an ion that does not naturally allow to pass through. In some cases, the mutation converts the permeability from cation to anion, for example the replacement of amino acid residues 260-281 in human α7-nAChR (CHRNA7) (EKISLGITVLLSLTVFMLLVAE, SEQ ID NO: 34) or the corresponding amino acids in another cation-permeable LGIC with the peptide sequence PAKIGLGITVLLSLTTFMSGVAN (SEQ ID NO: 35). In some cases, the mutation converts the permeability from anion to cation, for example, the substitution of amino acid residue 279 of GLRA1 or the corresponding amino acid in another anion-permeable LGIC to glutamic acid (E), (which, as an A293E substitution in GLRA1 converts the LGIC from being anion-permissive to calcium-permissive), or the deletion of amino acid residue 278 of GLRA1 or the corresponding amino acid in another anion-permeable LGIC, the substitution of amino acid residue 279 of GLRA1 or the corresponding amino acid in another anion-permeable LGIC to glutamic acid (E), and the substitution of amino acid residue 293 of GLRA1 or the corresponding amino acid in another anion-permeable LGIC to valine (V) (which, as a P278A, A279E, T293V in GLRA1 converts the LGIC from being anion-permissive to cation-permissive).

Additional engineered receptors beyond those described herein can be identified by in vitro screening and validation methods. In some embodiments, a library of parental receptor mutants is generated from a limited number of parental receptors. The parental receptors can be mutated using methods known in the art, including error prone PCR. In some embodiments, the library of parental receptor mutants is then transfected into yeast or mammalian cells and screened in high throughput to identify functional receptors (e.g., to identify parental receptor mutants that are capable of signaling in response to a ligand). In some embodiments, the functional parental receptor mutants identified in this primary screen is then expressed in mammalian cells and screened for potency to ligands, e.g., by the plate reader and/or electrophysiology assays described herein. The parental receptor mutants that demonstrate either increased binding affinity for agonist ligands, or that enable the use of antagonist or modulator ligands as agonists in the secondary screen can then be selected and carried though further in vitro and/or in vivo validation and characterization assays. Such screening assays are known in the art, for example Armbruster, B. N. et al. (2007) PNAS, 104, 5163-5168; Nichols, C. D. and Roth, B. L. (2009) Front. Mol. Neurosci. 2, 16; Dong, S. et al. (2010) Nat. Protoc. 5, 561-573; Alexander, G. M. et al. (2009) Neuron 63, 27-39; Guettier, J. M. et al. (2009) PNAS 106, 19197-19202; Ellefson J. W. et al. (2014) Nat Biotechnol. 32(1):97-101; Maranhao A C and Ellington A D. (2017) ACS Synth Biol. 20; 6(1):108-119; Talwar S et al. (2013) PLoS One; 8(3):e58479; Gilbert D. F. et al. (2009) Front Mol Neurosci. 30; 2:17; Lynagh and Lynch, (2010), Biol Chem. 14:285(20), 14890-14897; Islam R. et al. (2016) ACS Chem Neurosci. 21; 7(12):1647-1657; and Myers et al. (2008) Neuron. 8:58(3): 362-373.

A summary of these exemplary engineered receptors is provided below in Table 5. Each of these receptors comprises or consists of the amino acid sequence of SEQ ID NO: 33 except the indicated amino acid mutation.

TABLE 5
Exemplary Engineered Receptors

| Name | Sequence |
|---|---|
| CODA71 | SEQ ID NO: 33 |
| CODA536 | L131D, S172D in SEQ ID NO: 33 |
| CODA534 | L131S, S172D in SEQ ID NO: 33 |
| CODA535 | L131T, S172D in SEQ ID NO: 33 |
| CODA807 | Y115D, L131E in SEQ ID NO: 33 |
| CODA806 | Y115D, L131Q in SEQ ID NO: 33 |
| CODA805 | Y115D, S170T in SEQ ID NO: 33 |
| CODA952 | Y140I in SEQ ID NO: 33 |
| CODA1025 | R101F, L131G in SEQ ID NO: 33 |
| CODA1027 | R101F, L131D in SEQ ID NO: 33 |
| CODA1039 | Y115E, Y210W in SEQ ID NO: 33 |
| CODA1045 | R101W, Y210V in SEQ ID NO: 33 |
| CODA1047 | R101F, Y210V in SEQ ID NO: 33 |
| CODA1048 | R101F, Y210F in SEQ ID NO: 33 |
| CODA1053 | R101M, L131A in SEQ ID NO: 33 |
| CODA1054 | R101M, L131F in SEQ ID NO: 33 |
| CODA1055 | R101W, Y115E, Y210W in SEQ ID NO: 33 |
| CODA1056 | R101F, Y115E, Y210W in SEQ ID NO: 33 |
| CODA1138 | W77F, R101F, L131D in SEQ ID NO: 33 |
| CODA1140 | R101F, L13IN, S172D in SEQ ID NO: 33 |
| CODA1157 | Q139E, S172D in SEQ ID NO: 33 |
| CODA1173 | S172D, Y210W in SEQ ID NO: 33 |
| CODA965 | Y140C in SEQ ID NO: 33 |

### Ligands

In some embodiments, the ligands of the disclosure refer to exogenous drugs or compounds with a known mechanism of action on a mammalian cell (e.g., are known to act as an agonist, antagonist, or modulator of a receptor). Such ligands may also be referred to as “binding agents”. Ligands of the disclosure can include proteins, lipids, nucleic acids, and/or small molecules. In some embodiments, ligands include drugs or compounds that have been approved by the US Food and Drug Administration (FDA) for clinical use in the treatment of a particular disease (e.g., a neurological disease). In some embodiments, ligands include drugs or compounds that have not been approved by the FDA for clinical use, but have been tested in one or more clinical trials, are currently being tested in one or more clinical trials, and/or are anticipated to be tested in one or more clinical trials. In some embodiments, ligands include drugs or compounds that have not been approved by the FDA for clinical use, but are routinely used in laboratory research. In some embodiments, the ligand is an analog of one of the aforementioned ligands. In some embodiments, the ligand is selected from the group consisting of AZD0328, ABT-126, AQW-051, Cannabidiol, Cilansetron, PH-399733, FACINICLINE/RG3487/MEM-3454, TC-6987, and TC-5619/AT-101. In some embodiments, the ligand is selected from the group consisting of ABT-126, AZD-0328, RG3487, TC-6987, TC-6683, Varenicline, and TC-5619. In some embodiments, the ligand is TC-5619.

In particular embodiments, the ligand is an analog of Cilansetron, e.g., as described by one of the compound formulas 2-7 below in either its R or S enantiomer:

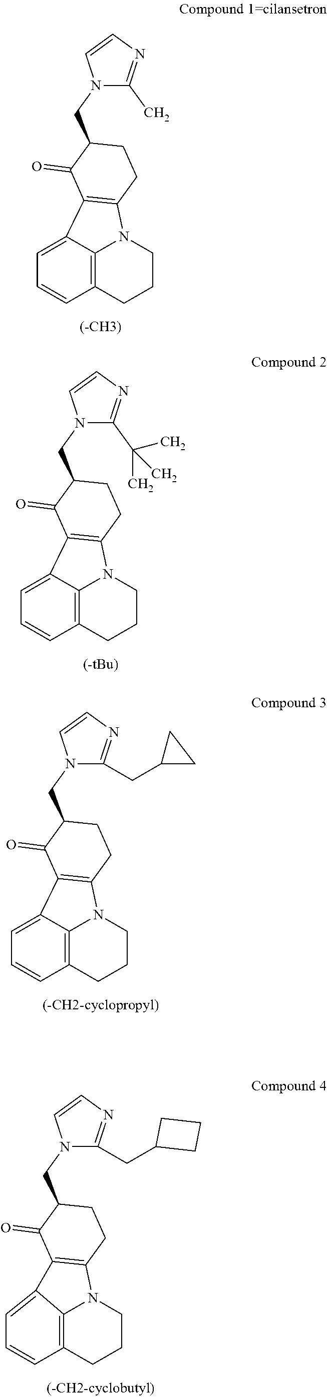 [SMILES: Cc1nccn1C[C@H]1CCc2c(c3cccc4c3n2CCC4)C1=O ; CC(C)(C)c1nccn1C[C@H]1CCc2c(c3cccc4c3n2CCC4)C1=O ; O=C1c2c(n3c4c(cccc24)CCC3)CC[C@@H]1Cn1ccnc1CC1CC1 ; O=C1c2c(n3c4c(cccc24)CCC3)CC[C@@H]1Cn1ccnc1CC1CCC1]

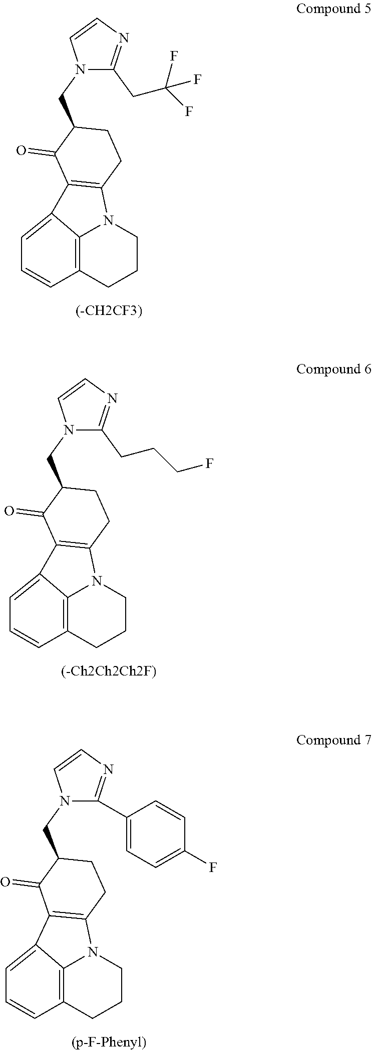 [SMILES: O=C1c2c(n3c4c(cccc24)CCC3)CC[C@@H]1Cn1ccnc1CC(F)(F)F ; O=C1c2c(n3c4c(cccc24)CCC3)CC[C@@H]1Cn1ccnc1CCCF ; O=C1c2c(n3c4c(cccc24)CCC3)CC[C@@H]1Cn1ccnc1-c1ccc(F)cc1]

In some embodiments, the ligand acts as an agonist. The term “agonist” as used herein refers to a ligand that induces a signaling response. In some embodiments, the ligand acts as an antagonist. The term antagonist is used herein to refer to a ligand that inhibits a signaling response.

In some embodiments, the ligand is AZD-0328 according to the formula below:

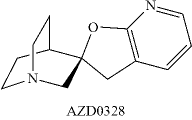 [SMILES: c1cnc2c(c1)C[C@@]1(CN3CCC1CC3)O2]

In some embodiments, the ligand is TC-6987 according to the formula below:

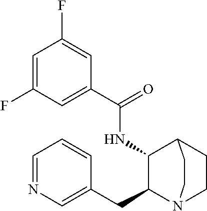 [SMILES: O=C(N[C@@H]1C2CCN(CC2)[C@H]1Cc1cccnc1)c1cc(F)cc(F)c1]

In some embodiments, the ligand is ABT-126 according to the formula below:

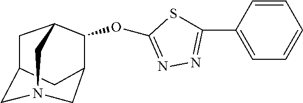 [SMILES: c1ccc(-c2nnc(O[C@@H]3C4CC5C[C@H]3C[N@](C5)C4)s2)cc1]

In some embodiments, the ligand is TC-5619/Bradanicline according to the formula below:

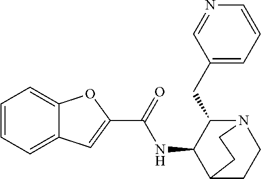 [SMILES: O=C(N[C@@H]1C2CCN(CC2)[C@H]1Cc1cccnc1)c1cc2ccccc2o1]

In some embodiments, the ligand is TC-6683 according to the formula below:

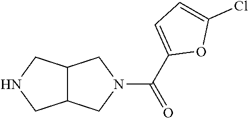 [SMILES: O=C(c1ccc(Cl)o1)N1CC2CNCC2C1]

In some embodiments, the ligand is Varenicline according to the formula below:

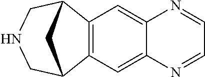 [SMILES: c1cnc2cc3c(cc2n1)[C@@H]1CNC[C@H]3C1]

In some embodiments, the ligand is Facinicline/RG3487 according to the formula below:

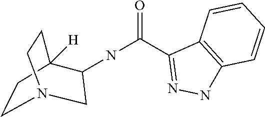 [SMILES: O=C(NC1CN2CCC1CC2)c1n[nH]c2ccccc12]

Exemplary combinations of engineered receptors described herein and non-native ligands are provided in Table 6 below. Each of the engineered receptors in Table 6 may be present as a protein, a polynucleotide encoding the protein, or a vector comprising the polynucleotide encoding the protein. In some embodiments, the engineered receptor comprises a ligand binding domain derived from human α7-nAChR. In some embodiments, the engineered receptor comprises an ion pore domain derived from human Glycine receptor. In some embodiments, the human Glycine receptor is human Glycine receptor cal. In some embodiments, the engineered receptor comprises a polypeptide sequence having at least 85%, at least 90%, at least 95%, at least 96%, at least 97%, at least 98%, at least 99%, at least 99.5%, or 100% identical to SEQ ID NO: 33 except for the indicated mutation in Table 6.

TABLE 6
Non-Limiting Examples of Engineered Receptor
and Non-Native Ligand Combination

| Engineered Receptor | | |
|---|---|---|
| with Amino Acid | | |
| Substitution(s) | | Non-limiting |
| Corresponding to the | | Example of the |
| Indicated Position(s) | Non-native | Engineered |
| in SEQ ID NO: 25 | ligand | Receptor |
| L131D_S172D | AZD-0328 | CODA536 |
| L131D_S172D | Facinicline | CODA536 |
| Y115D_S170T | Facinicline | CODA805 |
| Y115D_S170T | TC-6987 | CODA805 |
| Y115D_L131Q | AZD-0328 | CODA806 |
| Y115D_L131Q | Facinicline | CODA806 |
| Y115D_L131Q | TC-6987 | CODA806 |
| Y115D_L131E | TC-5619 | CODA807 |
| Y115D_L131E | AZD-0328 | CODA807 |
| Y115D_L131E | Facinicline | CODA807 |
| Y115D_L131E | TC-6987 | CODA807 |
| Y140I | TC-5619 | CODA952 |
| Y140I | Facinicline | CODA952 |
| Y140C | TC-5619 | CODA965 |
| Y140C | ABT-126 | CODA965 |
| Y140C | TC-6987 | CODA965 |
| R101F_L131G | TC-5619 | CODA1025 |
| R101F_L131G | AZD-0328 | CODA1025 |
| R101F_L131G | TC-6987 | CODA1025 |
| R101F_L131G | Varenicline | CODA1025 |
| R101F_L131D | TC-5619 | CODA1027 |
| R101F_L131D | TC-6987 | CODA1027 |
| Y115E_Y210W | TC-5619 | CODA1039 |
| Y115E_Y210W | ABT-126 | CODA1039 |
| R101W_Y210V | TC-5619 | CODA1045 |
| R101F_Y210V | TC-5619 | CODA1047 |
| R101F_Y210F | TC-5619 | CODA1048 |
| R101M_L131A | TC-5619 | CODA1053 |
| R101M_L131A | Varenicline | CODA1053 |
| R101M_L131F | TC-5619 | CODA1054 |
| R101M_L131F | Varenicline | CODA1054 |
| R101W_Y115E_Y210W | TC-5619 | CODA1055 |
| R101F_Y115E_Y210W | TC-5619 | CODA1056 |
| W77F_R101F_L131D | ABT-126 | CODA1138 |
| S172D_Y210W | ABT-126 | CODA1173 |

### Compositions

The disclosure also provides compositions (e.g., pharmaceutical compositions). In some embodiments, the composition or pharmaceutical composition comprise a recombinant nucleic acid comprising the expression cassette of the disclosure. In some embodiments, the expression cassette comprises a transgene encoding the engineered receptor (e.g., ligand-gated ion channel) of the disclosure. In some embodiments, the transgene encodes SEQ ID NO: 33 or a mutant thereof. In some embodiments, the composition or pharmaceutical composition comprise a pharmaceutically acceptable carrier, diluent, excipient, and/or buffer. In some embodiments, the pharmaceutically acceptable carrier, diluent, excipient, and/or buffer is suitable for use in a human.

Such excipients, carriers, diluents, and/or buffers (together as “pharmaceutical vehicles”) include any pharmaceutical agent that can be administered without undue toxicity. They may be approved by a regulatory agency of the Federal or a state government or listed in the U.S. Pharmacopeia or other generally recognized pharmacopeia for use in mammals, such as humans. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compounds and compositions of the disclosure and pharmaceutically acceptable vehicles, excipients, or diluents may be sterile. In some instances, an aqueous medium is employed as a vehicle when the compound of the disclosure is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro, (2000) Remington: The Science and Practice of Pharmacy, 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

In some embodiments, the pharmaceutical composition comprises a liquid comprising the recombinant nucleic acid in solution, in suspension, or both. As used herein, liquid compositions include gels. In some embodiments, the liquid composition is aqueous. In some embodiments, the composition is an in situ gellable aqueous composition, e.g., an in situ gellable aqueous solution.

Non-limiting examples of compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

In some embodiments, the pharmaceutical compositions are formulated to be compatible with a particular route of administration or delivery. Thus, pharmaceutical compositions include carriers, diluents, and/or excipients suitable for administration by various routes.

The choice of excipient will be determined in part by the particular vector, as well as by the particular method used to administer the composition. Accordingly, there is a wide variety of suitable formulations of the pharmaceutical composition of the present disclosure.

For example, the vector may be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Compositions suitable for parenteral administration comprise aqueous and non-aqueous solutions, suspensions or emulsions of the active compound. Preparations are typically sterile and can be isotonic with the blood of the intended recipient. Non-limiting illustrative examples include water, saline, dextrose, fructose, ethanol, animal, vegetable or synthetic oils.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

In some embodiments, the pharmaceutical compositions of the disclosure are suitable for parenteral administration, for example as aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

As another example, a compound may be formulated into a preparation suitable for oral administration, including (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The term “unit dosage form,” as used herein, refers to physically discrete units suitable as unitary dosages for human and animal subjects, each unit containing a predetermined quantity of compounds of the present disclosure calculated in an amount sufficient to produce the desired effect in association with a pharmaceutically acceptable diluent, carrier or vehicle. The specifications for the novel unit dosage forms of the present disclosure depend on the particular compound employed and the effect to be achieved, and the pharmacodynamics associated with each compound in the host.

### Methods

In some aspects of the disclosure, the compositions and methods disclosed herein can be utilized to treat a neurological disease or disorder in a subject in need thereof. In some aspects, the compositions and methods disclosed herein can be utilized to express a transgene in a neuron. In some aspects, the compositions and methods disclosed herein can be utilized in assays. In some aspects, the compositions and methods disclosed herein can be utilized as a research tool. In some aspects, the compositions and methods disclosed herein can be utilized as nucleic acid delivery tools.

In some aspects of the disclosure, a method of treating a neurological disease or disorder in a subject is provided, the method comprises administering an effective amount of a recombinant nucleic acid comprising an expression cassette of the disclosure to a subject. In some embodiments, the recombinant nucleic acid is delivered to a hippocampal neuron. In some embodiments, the recombinant nucleic acid is delivered to a dorsal root ganglion neuron or a trigeminal ganglion neuron. In some embodiments, the expression cassette comprises a transgene. In some embodiments, the transgene encodes an engineered LGIC. In some embodiments, the method further comprises administering a small molecule ligand/drug/agonist that activates the engineered LGIC, which in turn modulates the activity of the target neuron, and thereby treating the neurological diseases or disorder (e.g., pain or epilepsy) in the subject.

In some aspects of the disclosure, a method of treating focal epilepsy in a subject is provided, the method comprises administering an effective amount of a recombinant nucleic acid comprising an expression cassette to the subject, wherein the expression cassette comprises a transgene encoding an engineered LGIC receptor of the disclosure. In some embodiments, the recombinant nucleic acid is delivered into a hippocampal neuron. In some embodiments, the method further comprises administering a small molecule ligand that activates the engineered LGIC, which in turn modulates the activity of the target neuron, and thereby treating the focal epilepsy in the subject.

In some aspects, vectors, recombinant nucleic acids, or compositions disclosed herein are used in the manufacture of a medicament for treating a neurological disease or disorder. In some aspects, vectors, recombinant nucleic acids, or compositions disclosed herein are used in the manufacture of a medicament for treating a neurological disease or disorder associated with hippocampal neuron dysregulation, such as focal epilepsy, schizophrenia, autism spectrum disorder, Alzheimer's disease, Rett syndrome, and fragile X syndrome. In some aspects, vectors, recombinant nucleic acids, or compositions disclosed herein are used for treating focal epilepsy. In some aspects, vectors, recombinant nucleic acids, or compositions disclosed herein are used for treating schizophrenia. In some aspects, vectors, recombinant nucleic acids, or compositions disclosed herein are used for treating autism spectrum disorder. In some aspects, vectors, recombinant nucleic acids, or compositions disclosed herein are used for treating Alzheimer's disease. In some aspects, vectors, recombinant nucleic acids, or compositions disclosed herein are used for treating Rett syndrome. In some aspects, vectors, recombinant nucleic acids, or compositions disclosed herein are used for treating fragile X syndrome.

The present disclosure contemplates, in part, compositions and methods for controlling, managing, preventing, or treating epilepsy in a subject. In some embodiments, the epilepsy is focal epilepsy. In some embodiments, the focal epilepsy is mesial temporal lobe epilepsy (mTLE).

In some embodiments, the compositions and methods herein may be utilized to ameliorate the level of epileptic seizure in a subject. In some embodiments, the compositions and methods herein may be utilized to prevent or control the level of epileptic seizure in a subject. Epileptic seizures may be classified as tonic-clonic, tonic, clonic, myoclonic, absence or atonic seizures.

In some embodiments, the compositions and methods herein may reduce the number or frequency of epileptic seizures experienced by a subject by about 5%, about 10%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or 100%, including all ranges and subranges therebetween. In some embodiments, the compositions and methods herein may reduce the number or frequency of epileptic seizures experienced by a subject by at least 5%, at least 10%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or 100%, including all ranges and subranges therebetween.

In some embodiments, the compositions and methods herein may reduce the level and/or duration of epileptic seizures experienced by a subject by about 5%, about 10%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or 100%, including all ranges and subranges therebetween. In some embodiments, the compositions and methods herein may reduce the level and/or duration of epileptic seizures experienced by a subject by at least 5%, at least 10%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or 100%, including all ranges and subranges therebetween.

In various embodiments, a method for controlling, managing, preventing, or treating epilepsy (e.g., focal epilepsy) in a subject comprises administering to the subject an effective amount of a recombinant nucleic acid comprising an expression cassette of the disclosure. Without wishing to be bound by any particular theory, the present disclosure contemplates using the expression cassette comprising a transgene encoding an engineered LGIC disclosed herein to modulate neuronal activity to alleviate epilepsy in the subject.

In various embodiments, a vector and/or recombinant nucleic acid comprising an expression cassette encoding an engineered receptor that activates or depolarizes neuronal cells is administered to (or introduced into) one or more neuronal cells that controls epilepsy. In the presence of ligand the neuronal cell expressing the engineered receptor, is activated and decreases the sensitivity to epilepsy.

In some embodiments, the epilepsy is focal epilepsy (focal epilepsy seizures). Focal epilepsy is a neurological condition in which the predominant symptom is recurring seizures that affect one hemisphere (half) of the brain.

Focal epilepsy come in four categories: (a) Focal aware seizures, in which the subject is aware of what's happening during the seizure; (b) Focal impaired awareness seizures; in which the subject is confused or don't know what's happening during the seizure or don't remember it; (c) Focal motor seizures, in which the subject moves to some extent—anything from twitching, to spasms, to rubbing hands, to walking around; and (d) Focal non-motor seizures, in which the subject does not twitch or make other movements during seizure. Instead, it causes changes in how the subject feels or thinks (e.g., feeling intense emotions, strange feelings, or symptoms like a racing heart, goose bumps, or waves of heat or cold).

Focal epilepsies are characterized by seizures arising from a specific part (lobe) of the brain. Focal epilepsies include idiopathic location-related epilepsies (ILRE), frontal lobe epilepsy, temporal lobe epilepsy, parietal lobe epilepsy and occipital lobe epilepsy.

Idiopathic Localization-Related Epilepsies (ILRE) is caused by unknown factors.

Frontal Lobe Epilepsy is the term for recurring seizures beginning in the frontal lobe—the area of the brain behind the forehead.

Temporal lobe epilepsy is the term for recurring seizures beginning in the temporal lobe—the section of the brain located on the sides of the head behind the temples and cheekbones. The temporal lobes are the areas of the brain that most commonly give rise to seizures. The mesial portion (middle) of both temporal lobes is very important in epilepsy it is frequently the source of seizures and can be prone to damage or scarring. Mesial temporal lobe epilepsy (mTLE) is the most common form of human epilepsy. Often times, its pathophysiological substrate is hippocampal sclerosis. Thus, one strategy of treating focal epilepsy such as mTLE is by targeting hippocampal neurons.

Parietal Lobe Epilepsy. The parietal lobe is the section of the brain on the top and sides of the head. Known as the “association cortex,” the parietal lobe is responsible for connecting meaning to the brain's functions. It is here that the brain creates a visual image, that sounds are recognized as words, and that the sense of touch is associated with a particular object. In some ways, the parietal lobe is where perception meshes with physical reality.

Occipital Lobe Epilepsy. Occipital lobe epilepsy is the term for recurring seizures beginning in the occipital lobe, the section of the brain in the back of the head that is primarily responsible for vision.

In some aspects of the disclosure, a method of treating neuropathic pain, such as Peripheral Neuropathy and Trigeminal Neuralgia, in a subject is provided, the method comprises administering an effective amount of a recombinant nucleic acid to the subject, wherein the recombinant nucleic acid comprises a expression cassette comprising a transgene encoding an engineered LGIC receptor of the disclosure. In some embodiments, the recombinant nucleic acid is delivered into a DRG or TGG neuron. In some embodiments, the method further comprises administering a small molecule ligand that activates the engineered LGIC, which in turn modulates the activity of the target neuron, and thereby treating neuropathic pain, such as Peripheral Neuropathy and Trigeminal Neuralgia, in the subject.

In some aspects, vectors, recombinant nucleic acids, or compositions disclosed herein are used in the manufacture of a medicament for treating a neurological disease or disorder. In some aspects, vectors, recombinant nucleic acids, or compositions disclosed herein are used in the manufacture of a medicament for treating a neurological disease or disorder associated with DRG/TGG neuron dysregulation, such as neuropathic pain. In some aspects, vectors, recombinant nucleic acids, or compositions disclosed herein are used in the manufacture of a medicament for treating a spinal cord related disease such as spasticity, spinal cord injury, and avulsion injury. In some aspects, vectors, recombinant nucleic acids, or compositions disclosed herein are used for treating spasticity. In some embodiments, treating spasticity comprises transducing neurons in the spinal column. In some aspects, vectors, recombinant nucleic acids, or compositions disclosed herein are used for treating spinal cord injury. In some aspects, vectors, recombinant nucleic acids, or compositions disclosed herein are used for treating avulsion injury. In some embodiments, treating spinal cord injury or avulsion injury comprises transducing a neuron in the dorsal horn.

The present disclosure contemplates, in part, compositions and methods for controlling, managing, preventing, or treating pain in a subject. In some embodiments, the pain is neuropathic pain. “Pain” refers to an uncomfortable feeling and/or an unpleasant sensation in the body of a subject. Feelings of pain can range from mild and occasional to severe and constant. Pain can be classified as acute pain or chronic pain. Pain can be nociceptive pain (i.e., pain caused by tissue damage), neuropathic pain or psychogenic pain. In some cases, the pain is caused by or associated with a disease (e.g., cancer, arthritis, diabetes). In other cases, the pain is caused by injury (e.g., sports injury, trauma). Non-limiting examples of pain that are amenable to treatment with the compositions and methods herein include: neuropathic pain including peripheral neuropathy, diabetic neuropathy, post herpetic neuralgia, trigeminal neuralgia, back pain, neuropathy associated with cancer, neuropathy associated with HIV/AIDS, phantom limb pain, carpal tunnel syndrome, central post-stroke pain, pain associated with chronic alcoholism, hypothyroidism, uremia, pain associated with multiple sclerosis, pain associated with spinal cord injury, pain associated with Parkinson's disease, epilepsy, osteoarthritic pain, rheumatoid arthritic pain, visceral pain, and pain associated with vitamin deficiency; and nociceptive pain including pain associated with central nervous system trauma, strains/sprains, and burns; myocardial infarction, acute pancreatitis, post-operative pain, posttraumatic pain, renal colic, pain associated with cancer, pain associated with fibromyalgia, pain associated with carpal tunnel syndrome, and back pain.

The compositions and methods herein may be utilized to ameliorate a level of pain in a subject. In some cases, a level of pain in a subject is ameliorated by at least at least 5%, at least at least 10%, at least at least 15%, at least at least 20%, at least at least 25%, at least at least 30%, at least at least 35%, at least 40%, at least 45%, at least 50%, at least 55%, at least 60%, at least 65%, at least 70%, at least 75%, at least 80%, at least 85%, at least 90%, at least 95%, at least 99% or about 100%, including all ranges and subranges therebetween. A level of pain in a subject can be assessed by a variety of methods. In some cases, a level of pain is assessed by self-reporting (i.e., a human subject expresses a verbal report of the level of pain he/she is experiencing). In some cases, a level of pain is assessed by behavioral indicators of pain, for example, facial expressions, limb movements, vocalization, restlessness and guarding. These types of assessments may be useful for example when a subject is unable to self-report (e.g., an infant, an unconscious subject, a non-human subject). A level of pain may be assessed after treatment with a composition of the disclosure as compared to the level of pain the subject was experiencing prior to treatment with the composition.

In various embodiments, a method for controlling, managing, preventing, or treating pain in a subject comprises administering to the subject an effective amount of a recombinant nucleic acid comprising an expression cassette encoding an engineered LGIC of the disclosure. Without wishing to be bound by any particular theory, the present disclosure contemplates using the vectors and/or recombinant nucleic acids encoding an engineered LGIC disclosed herein to modulate neuronal activity to alleviate pain in the subject.

In various embodiments, a recombinant nucleic acid comprising an expression cassette encoding an engineered receptor that activates or depolarizes neuronal cells is administered to (or introduced into) one or more neuronal cells that decrease pain sensation, e.g., inhibitory interneurons. In the presence of ligand the neuronal cell expressing the engineered receptor, is activated and decreases the sensitivity to pain potentiating the analgesic effect of stimulating these neuronal cells.

In various embodiments, a recombinant nucleic acid comprising an expression cassette encoding an engineered receptor that deactivates or hyperpolarizes neuronal cells is administered to (or introduced into) one or more neuronal cells that increase pain sensation or sensitivity to pain, e.g., nociceptor, peripheral sensory neurons, C-fibers, Aδ fibers, Aβ fibers, DRG neurons, TGG neurons, and the like. In the presence of ligand the neuronal cell expressing the engineered receptor, is deactivated and decreases the sensitivity to pain and potentiating an analgesic effect.

In some embodiments, the compositions and methods of the disclosure are effective in reducing pain. Illustrative examples of pain that are amenable to treatment with the compositions, and methods of the disclosure, include but are not limited to acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritic pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, and menstrual pain.

Pain can be classified as acute or chronic. “Acute pain” refers to pain that begins suddenly and is usually sharp in quality. Acute pain might be mild and last just a moment, or it might be severe and last for weeks or months. In most cases, acute pain does not last longer than three months, and it disappears when the underlying cause of pain has been treated or has healed. Unrelieved acute pain, however, may lead to chronic pain. “Chronic pain” refers to ongoing or recurrent pain, lasting beyond the usual course of acute illness or injury or lasting for more than three to six months, and which adversely affects the individual's well-being. In some embodiments, the term “chronic pain” refers to pain that continues when it should not. Chronic pain can be nociceptive pain or neuropathic pain.

In some embodiments, the pain is expected or anticipated to develop in association with or as a result of an injury, an infection, or a medical intervention. In some embodiments, the infection causes nerve damage. In some embodiments, the medical intervention is a surgery, such as surgery to the central core of the body. In some embodiments, the medical intervention is a surgery to remove parts or whole of one or more tissues, tumors or organs in the body. In some embodiments, the medical intervention is an amputation. In some embodiments, the compositions and methods of the disclosure are effective in reducing acute pain. In some embodiments, the compositions and methods of the disclosure are effective in reducing chronic pain.

Clinical pain is present when discomfort and abnormal sensitivity feature among the patient's symptoms. Individuals can present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia-Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive pain, inflammatory pain, and neuropathic pain.

In some embodiments, the compositions and methods of the disclosure are effective in reducing nociceptive pain. In some embodiments, the compositions and methods of the disclosure are effective in reducing inflammatory pain. In some embodiments, the compositions and methods of the disclosure are effective in reducing neuropathic pain.

Nociceptive pain is induced by tissue injury or by intense stimuli with the potential to cause injury. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g., bone pain, headache, facial pain or visceral pain) or pain associated with cancer therapy (e.g., post chemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain can be defined as pain initiated or caused by a primary lesion or dysfunction in the nervous system. Etiologies of neuropathic pain include, e.g., peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy, and vitamin deficiency.

Neuropathic pain can be related to a pain disorder, a term referring to a disease, disorder or condition associated with or caused by pain. Illustrative examples of pain disorders include arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

In some embodiments, the neuropathic pain is peripheral neuropathy. Peripheral neuropathy refers to the conditions that result when nerves that carry messages to and from the brain and spinal cord from and to the rest of the body are damaged or diseased. Various kinds of peripheral neuropathy range from carpal tunnel syndrome (a traumatic injury common after chronic repetitive use of the hands and wrists, such as with computer use) to nerve damage linked to diabetes. In general, peripheral neuropathy can be categorized into mononeuropathy and polyneuropathy. Mononeuropathy includes carpal tunnel syndrome, ulnar nerve palsy, radial nerve palsy, and peroneal nerve palsy. Polyneuropathy occurs when multiple peripheral nerves throughout the body malfunction at the same time. Polyneuropathy can have a wide variety of causes, including exposure to certain toxins such as with alcohol abuse, poor nutrition (particularly vitamin B deficiency), and complications from diseases such as cancer or kidney failure. One of the most common forms of chronic polyneuropathy is diabetic neuropathy, a condition that occurs in people with diabetes. It is more severe in people with poorly controlled blood sugar levels. Though less common, diabetes can also cause a mononeuropathy. One of the most serious polyneuropathies is Guillain-Barre syndrome, a rare disease that strikes suddenly when the body's immune system attacks nerves in the body just as they leave the spinal cord. Symptoms tend to appear quickly and worsen rapidly, sometimes leading to paralysis. Early symptoms include weakness and tingling that eventually may spread upward into the arms. Blood pressure problems, heart rhythm problems, and breathing difficulty may occur in the more severe cases. Chronic inflammatory demyelinating polyneuropathy (CIDP) is a chronic form of Guillain-Barre in which the symptoms continue for months and even years. Early diagnosis and treatment is crucial for CIDP patients, 30% of which risk eventually being confined to a wheelchair.

In some embodiments, the neuropathic pain is trigeminal neuralgia. Trigeminal neuralgia (TN), also called tic douloureux, is a chronic pain condition that affects the trigeminal or 5th cranial nerve, one of the most widely distributed nerves in the head. The trigeminal nerve is one set of the cranial nerves in the head. It is the nerve responsible for providing sensation to the face. One trigeminal nerve runs to the right side of the head, while the other runs to the left. Each of these nerves has three distinct branches. After the trigeminal nerve leaves the brain and travels inside the skull, it divides into three smaller branches, controlling sensations throughout the face: Ophthalmic Nerve (V1): The first branch controls sensation in a person's eye, upper eyelid and forehead. Maxillary Nerve (V2): The second branch controls sensation in the lower eyelid, cheek, nostril, upper lip and upper gum. Mandibular Nerve (V3): The third branch controls sensations in the jaw, lower lip, lower gum and some of the muscles used for chewing.

TN is a form of neuropathic pain. The typical or “classic” form of the disorder (called “Type 1” or TN1) causes extreme, sporadic, sudden burning or shock-like facial pain that lasts anywhere from a few seconds to as long as two minutes per episode. These attacks can occur in quick succession, in volleys lasting as long as two hours. The “atypical” form of the disorder (called “Type 2” or TN2), is characterized by constant aching, burning, stabbing pain of somewhat lower intensity than Type 1. Both forms of pain may occur in the same person, sometimes at the same time. The intensity of pain can be physically and mentally incapacitating. TN is associated with a variety of conditions. TN can be caused by a blood vessel pressing on the trigeminal nerve as it exits the brain stem. This compression causes the wearing away or damage to the protective coating around the nerve (the myelin sheath). TN symptoms can also occur in people with multiple sclerosis, a disease that causes deterioration of the trigeminal nerve's myelin sheath. Rarely, symptoms of TN may be caused by nerve compression from a tumor, or a tangle of arteries and veins called an arteriovenous malformation. Injury to the trigeminal nerve (perhaps the result of sinus surgery, oral surgery, stroke, or facial trauma) may also produce neuropathic facial pain.

The inflammatory process is a complex series of biochemical and cellular events, activated in response to tissue injury or the presence of foreign substances, which results in swelling and pain. Arthritic pain is a common inflammatory pain.

Other types of pain that are amenable to treatment with the compositions, and methods of the disclosure, include but are not limited to pain resulting from musculoskeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome, and temporomandibular myofascial pain.

The effective amount of the compositions and methods of the disclosure to reduce the amount of pain experienced by a human subject can be determined using a variety of pain scales. Patient self-reporting can be used to assess whether pain is reduced; see, e.g., Katz and Melzack (1999) Surg. Clin. North Am. 79:231. Alternatively, an observational pain scale can be used. The LANSS Pain Scale can be used to assess whether pain is reduced; see, e.g., Bennett (2001) Pain 92:147. A visual analog pain scale can be used; see, e.g., Schmader (2002) Clin. J. Pain 18:350. The Likert pain scale can be used; e.g., where 0 is no pain, 5 is moderate pain, and 10 is the worst pain possible. Self-report pain scales for children include, e.g., Faces Pain Scale; Wong-Baker FACES Pain Rating Scale; and Colored Analog Scale. Self-report pain scales for adults include, e.g., Visual Analog Scale; Verbal Numerical Rating Scale; Verbal Descriptor Scale; and Brief Pain Inventory. Pain measurement scales include, e.g., Alder Hey Triage Pain Score (Stewart et al. (2004) Arch. Dis. Child. 89:625); Behavioral Pain Scale (Payen et al. (2001) Critical Care Medicine 29:2258); Brief Pain Inventory (Cleeland and Ryan (1994) Ann. Acad. Med. Singapore 23: 129); Checklist of Nonverbal Pain Indicators (Feldt (2000) Pain Manag. Nurs. 1: 13); Critical-Care Pain Observation Tool (Gelinas et al. (2006) Am. J. Crit. Care 15:420); COMFORT scale (Ambuel et al. (1992) J. Pediatric Psychol. 17:95); Dallas Pain Questionnaire (Ozguler et al. (2002) Spine 27:1783); Dolorimeter Pain Index (Hardy et al. (1952) Pain Sensations and Reactions Baltimore: The Williams & Wilkins Co.); Faces Pain Scale—Revised (Hicks et al. (2001) Pain 93:173); Face Legs Activity Cry Consolability Scale; McGill Pain Questionnaire (Melzack (1975) Pain 1:277); Descriptor Differential Scale (Gracely and Kwilosz (1988) Pain 35:279); Numerical 1 1 point Box (Jensen et al. (1989) Clin. J. Pain 5: 153); Numeric Rating Scale (Hartrick et al. (2003) Pain Pract. 3:310); Wong-Baker FACES Pain Rating Scale; and Visual Analog Scale (Huskisson (1982) J. Rheumatol. 9:768).

In some embodiments, a method of relieving pain in a subject is provided, the method comprising administering a recombinant nucleic acid comprising an expression cassette of the disclosure, which introduce an engineered LGIC encoded by a transgene into a neuronal cell and controlling the activity of the cell by providing an effective amount of a ligand that activates the engineered receptor, thereby relieving pain in the subject. The method provides significant analgesia for pain without off-target effects, such as general central nervous system depression. In certain embodiments, the method provides at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more (including all ranges and subranges therebetween) reduction in the neuropathic pain in a subject compared to an untreated subject. In some embodiments, the method comprises the step of measuring pain in the subject before and after the administration of the ligand, wherein the pain in the subject is reduced by at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or more, including all ranges and subranges therebetween. In such instances, the measuring may occur 4 hours or more after administration of the ligand, e.g., 8 hours 12 hours, 16 hours, 24 hours, 36 hours, 48 hours, 3 days, or 4 days or more after administration of the ligand.

In some cases, the compositions and methods are utilized to treat post-traumatic stress disorder (PTSD), gastroesophageal reflex disease (GERD), addiction (e.g., alcohol, drugs), anxiety, depression, memory loss, dementia, sleep apnea, stroke, urinary incontinence, narcolepsy, essential tremor, movement disorder, atrial fibrillation, cancer (e.g., brain tumors), Parkinson's disease, or Alzheimer's disease. Other non-limiting examples of neurological diseases or disorders that can be treated by the compositions and methods herein include: Abulia, Agraphia, Alcoholism, Alexia, Aneurysm, Amaurosis fugax, Amnesia, Amyotrophic lateral sclerosis (ALS), Angelman syndrome, Aphasia, Apraxia, Arachnoiditis, Arnold-Chiari malformation, Asperger syndrome, Ataxia, Ataxia-telangiectasia, Attention deficit hyperactivity disorder, Auditory processing disorder, Autism spectrum, Bipolar disorder, Bell's palsy, Brachial plexus injury, Brain damage, Brain injury, Brain tumor, Canavan disease, Capgras delusion, Carpal tunnel syndrome, Causalgia, Central pain syndrome, Central pontine myelinolysis, Centronuclear myopathy, Cephalic disorder, Cerebral aneurysm, Cerebral arteriosclerosis, Cerebral atrophy, Cerebral autosomal dominant arteriopathy with subcortical infarcts and leukoencephalopathy (CADASIL), Cerebral gigantism, Cerebral palsy, Cerebral vasculitis, Cervical spinal stenosis, Charcot-Marie-Tooth disease, Chiari malformation, Chorea, Chronic fatigue syndrome, Chronic inflammatory demyelinating polyneuropathy (CIDP), Chronic pain, Coffin-Lowry syndrome, Coma, Complex regional pain syndrome, Compression neuropathy, Congenital facial diplegia, Corticobasal degeneration, Cranial arteritis, Craniosynostosis, Creutzfeldt-Jakob disease, Cumulative trauma disorders, Cushing's syndrome, Cyclothymic disorder, Cytomegalic inclusion body disease (CIBD), Cytomegalovirus Infection, Dandy-Walker syndrome, Dawson disease, De Morsier's syndrome, Dejerine-Klumpke palsy, Dejerine-Sottas disease, Delayed sleep phase syndrome, Dementia, Dermatomyositis, Developmental coordination disorder, Diabetic neuropathy, Diffuse sclerosis, Diplopia, Down syndrome, Dravet syndrome, Duchenne muscular dystrophy, Dysarthria, Dysautonomia, Dyscalculia, Dysgraphia, Dyskinesia, Dyslexia, Dystonia, Empty sella syndrome, Encephalitis, Encephalocele, Encephalotrigeminal angiomatosis, Encopresis, Enuresis, Epilepsy, Epilepsy-intellectual disability in females, Erb's palsy, Erythromelalgia, Exploding head syndrome, Fabry's disease, Fahr's syndrome, Fainting, Familial spastic paralysis, Febrile seizures, Fisher syndrome, Friedreich's ataxia, Fibromyalgia, Foville's syndrome, Fetal alcohol syndrome, Fragile X syndrome, Fragile X-associated tremor/ataxia syndrome (FXTAS), Gaucher's disease, Generalized epilepsy with febrile seizures plus, Gerstmann's syndrome, Giant cell arteritis, Giant cell inclusion disease, Globoid Cell Leukodystrophy, Gray matter heterotopia, Guillain-Barre syndrome, Generalized anxiety disorder, HTLV-1 associated myelopathy, Hallervorden-Spatz disease, Head injury, Headache, Hemifacial Spasm, Hereditary Spastic Paraplegia, Heredopathia atactica polyneuritiformis, Herpes zoster oticus, Herpes zoster, Hirayama syndrome, Hirschsprung's disease, Holmes-Adie syndrome, Holoprosencephaly, Huntington's disease, Hydranencephaly, Hydrocephalus, Hypercortisolism, Hypoxia, Immune-Mediated encephalomyelitis, Inclusion body myositis, Incontinentia pigmenti, Infantile Refsum disease, Infantile spasms, Inflammatory myopathy, Intracranial cyst, Intracranial hypertension, Isodicentric 15, Joubert syndrome, Karak syndrome, Kearns-Sayre syndrome, Kinsbourne syndrome, Kleine-Levin Syndrome, Klippel Feil syndrome, Krabbe disease, Lafora disease, Lambert-Eaton myasthenic syndrome, Landau-Kleffner syndrome, Lateral medullary (Wallenberg) syndrome, Learning disabilities, Leigh's disease, Lennox-Gastaut syndrome, Lesch-Nyhan syndrome, Leukodystrophy, Leukoencephalopathy with vanishing white matter, Lewy body dementia, Lissencephaly, Locked-In syndrome, Lumbar disc disease, Lumbar spinal stenosis, Lyme disease—Neurological Sequelae, Machado-Joseph disease (Spinocerebellar ataxia type 3), Macrencephaly, Macropsia, Mal de debarquement, Megalencephalic leukoencephalopathy with subcortical cysts, Megalencephaly, Melkersson-Rosenthal syndrome, Menieres disease, Meningitis, Menkes disease, Metachromatic leukodystrophy, Microcephaly, Micropsia, Migraine, Miller Fisher syndrome, Mini-stroke (transient ischemic attack), Misophonia, Mitochondrial myopathy, Mobius syndrome, Monomelic amyotrophy, Motor skills disorder, Moyamoya disease, Mucopolysaccharidoses, Multi-infarct dementia, Multifocal motor neuropathy, Multiple sclerosis, Multiple system atrophy, Muscular dystrophy, Myalgic encephalomyelitis, Myasthenia gravis, Myelinoclastic diffuse sclerosis, Myoclonic Encephalopathy of infants, Myoclonus, Myopathy, Myotubular myopathy, Myotonia congenita, Narcolepsy, Neuro-Behget's disease, Neurofibromatosis, Neuroleptic malignant syndrome, Neurological manifestations of AIDS, Neurological sequelae of lupus, Neuromyotonia, Neuronal ceroid lipofuscinosis, Neuronal migration disorders, Neuropathy, Neurosis, Niemann-Pick disease, Non-24-hour sleep-wake disorder, Nonverbal learning disorder, O'Sullivan-McLeod syndrome, Occipital Neuralgia, Occult Spinal Dysraphism Sequence, Ohtahara syndrome, Olivopontocerebellar atrophy, Opsoclonus myoclonus syndrome, Optic neuritis, Orthostatic Hypotension, Otosclerosis, Overuse syndrome, Palinopsia, Paresthesia, Parkinson's disease, Paramyotonia Congenita, Paraneoplastic diseases, Paroxysmal attacks, Parry-Romberg syndrome, PANDAS, Pelizaeus-Merzbacher disease, Periodic Paralyses, Peripheral neuropathy, Pervasive developmental disorders, Photic sneeze reflex, Phytanic acid storage disease, Pick's disease, Pinched nerve, Pituitary tumors, PMG, Polyneuropathy, Polio, Polymicrogyria, Polymyositis, Porencephaly, Post-Polio syndrome, Postherpetic Neuralgia (PHN), Postural Hypotension, Prader-Willi syndrome, Primary Lateral Sclerosis, Prion diseases, Progressive hemifacial atrophy, Progressive multifocal leukoencephalopathy, Progressive Supranuclear Palsy, Prosopagnosia, Pseudotumor cerebri, Quadrantanopia, Quadriplegia, Rabies, Radiculopathy, Ramsay Hunt syndrome type I, Ramsay Hunt syndrome type II, Ramsay Hunt syndrome type III, Rasmussen encephalitis, Reflex neurovascular dystrophy, Refsum disease, REM sleep behavior disorder, Repetitive stress injury, Restless legs syndrome, Retrovirus-associated myelopathy, Rett syndrome, Reye's syndrome, Rhythmic Movement Disorder, Romberg syndrome, Saint Vitus dance, Sandhoff disease, Schilder's disease, Schizencephaly, Sensory processing disorder, Septo-optic dysplasia, Shaken baby syndrome, Shingles, Shy-Drager syndrome, Sjögren's syndrome, Sleep apnea, Sleeping sickness, Snatiation, Sotos syndrome, Spasticity, Spina bifida, Spinal cord injury, Spinal cord tumors, Spinal muscular atrophy, Spinal and bulbar muscular atrophy, Spinocerebellar ataxia, Split-brain, Steele-Richardson-Olszewski syndrome, Stiff-person syndrome, Stroke, Sturge-Weber syndrome, Stuttering, Subacute sclerosing panencephalitis, Subcortical arteriosclerotic encephalopathy, Superficial siderosis, Sydenham's chorea, Syncope, Synesthesia, Syringomyelia, Tarsal tunnel syndrome, Tardive dyskinesia, Tardive dysphrenia, Tarlov cyst, Tay-Sachs disease, Temporal arteritis, Temporal lobe epilepsy, Tetanus, Tethered spinal cord syndrome, Thomsen disease, Thoracic outlet syndrome, Tic Douloureux, Todd's paralysis, Tourette syndrome, Toxic encephalopathy, Transient ischemic attack, Transmissible spongiform encephalopathies, Transverse myelitis, Traumatic brain injury, Tremor, Trichotillomania, Trigeminal neuralgia, Tropical spastic paraparesis, Trypanosomiasis, Tuberous sclerosis, Unverricht-Lundborg disease, Von Hippel-Lindau disease (VHL), Viliuisk Encephalomyelitis (VE), Wallenberg's syndrome, West syndrome, Whiplash, Williams syndrome, Wilson's disease, or Zellweger syndrome.

A subject treated by methods and compositions disclosed herein can be a human, or a non-human animal. In some embodiments, the subject is a human. In some embodiments, the human is an adult (≥18-year-old). Non-limiting examples of non-human animals include a non-human primate, a livestock animal, a domestic pet, and a laboratory animal. For example, a non-human animal can be an ape (e.g., a chimpanzee, a baboon, a gorilla, or an orangutan), an old world monkey (e.g., a rhesus monkey), a new world monkey, a dog, a cat, a bison, a camel, a cow, a deer, a pig, a donkey, a horse, a mule, a lama, a sheep, a goat, a buffalo, a reindeer, a yak, a mouse, a rat, a rabbit, or any other non-human animal. The compositions and methods as described herein are amenable to the treatment of a veterinary animal. A veterinary animal can include, without limitation, a dog, a cat, a horse, a cow, a sheep, a mouse, a rat, a guinea pig, a hamster, a rabbit, a snake, a turtle, and a lizard. In some aspects, contacting the tissue or cell population with a composition comprises administering the composition to a cell population or subject. In some embodiments, administration occurs in vitro, for example by adding the composition to a cell culture system. In some aspects, administration occurs in vivo, for example by administration through a particular route. Wherein more than one composition is to be administered, the compositions may be administered via the same route at the same time (e.g., on the same day), or via the same route at different times. Alternatively, the compositions may be administered via different routes at the same time (e.g., on the same day) or via different routes at different times.

### Administration

The present disclosure provides dosing regimens for administering the recombinant nucleic acid comprising the expression cassette of the disclosure or the pharmaceutical compositions thereof. In some embodiments, the recombinant nucleic acid is incorporated in a vector (e.g., an AAV vector) for administration.

In some embodiments, the recombinant nucleic acid is administered or introduced into one or more neuronal cells. The neuronal cells may be the same type of neuronal cells, or a mixed population of different types of neuronal cells.

In some embodiments, the neuronal cell is a hippocampal neuron. In some embodiments, the neuronal cell is an excitatory neuron. In some embodiments, the recombinant nucleic acids are administered or introduced into one or more hippocampal neuronal cells.

In some embodiments, the neuronal cell is a nociceptor or peripheral sensory neuron. Illustrative examples of sensory neurons include, but are not limited to, dorsal root ganglion (DRG) neurons and trigeminal ganglion (TGG) neurons. In some embodiments, the neuronal cell is an inhibitory interneuron involved in the neuronal pain circuit. In some embodiments, the recombinant nucleic acid of the disclosure is administered or introduced into one or more DRG neuronal cells. In some embodiments, the recombinant nucleic acid of the disclosure is administered or introduced into one or more TGG neuronal cells.

Non-limiting examples of methods of administration include subcutaneous administration, intravenous administration, intramuscular administration, intradermal administration, intraperitoneal administration, oral administration, infusion, intracranial administration, intrathecal administration, intranasal administration, intraganglionic administration, intraspinal administration, Cisterna magna administration and intraneural administration. In some embodiments, administration can involve injection of a liquid formulation of the recombinant nucleic acid or a vector comprising the recombinant nucleic acid. In some embodiments, administration can involve oral delivery of a solid formulation of the vector or the recombinant nucleic acid. In some embodiments, the oral formulation can be administered with food. In some embodiments, the recombinant nucleic acid of the disclosure is parenterally, intravenously, intramuscularly, intraperitoneally, intrathecally, intraneurally, intraganglionicly, intraspinally, or intraventricularly administered to a subject in order to introduce the recombinant nucleic acid into one or more neuronal cells.

In some embodiments, the recombinant nucleic acid of the disclosure is administered to neurons intracranially, intrathecally (IT), intracerebrally, intraventricularly, or via direct injection into the epileptic focus in hippocampus.

In some embodiments, the recombinant nucleic acid of the disclosure is administered to sensory neuron or nociceptor, e.g., DRG neurons, TGG neurons, etc. by intrathecal (IT) or intraganglionic (IG) administration.

Intrathecal (IT) administration comprises delivery through the spine or through Cisterna magna. The IT route delivers the recombinant nucleic acid to the cerebrospinal fluid (CSF). This route of administration may be suitable for the treatment of e.g., chronic pain or other peripheral nervous system (PNS) or central nervous system (CNS) indications. In animals, IT administration has been achieved by inserting an IT catheter through the Cisterna magna and advancing it caudally to the lumbar level. In humans, IT delivery can be easily performed by lumbar puncture (LP), a routine bedside procedure with excellent safety profile.

In some embodiments, the recombinant nucleic acid of the disclosure is administered to a subject by intraganglionic administration. Intraganglionic administration may involve an injection directly into one or more ganglia. The IG route may deliver the recombinant nucleic acid directly into the DRG or TGG parenchyma. In some embodiments, IG administration to the DRG is performed by an open neurosurgical procedure. In some embodiments, the open neurosurgical procedure is invasive and not desirable in humans. In some embodiments, a minimally invasive, CT imaging-guided technique to safely target the DRG can be used, for example for human subjects. A customized needle assembly for convection enhanced delivery (CED) can be used to deliver the recombinant nucleic acid into the DRG parenchyma. In a non-limiting example, a recombinant nucleic acid of the disclosure may be delivered to one or more dorsal root ganglia and/or trigeminal ganglia for the treatment of chronic pain. In another non-limiting example, a recombinant nucleic acid of the disclosure may be delivered to the nodose ganglion (vagus nerve) to treat epilepsy.

In some embodiments, a recombinant nucleic acid of the disclosure is administered to the subject by intracranial administration (i.e., directly into the brain). In non-limiting examples of intracranial administration, a recombinant nucleic acid of the disclosure may be delivered into the cortex of the brain to treat e.g., an epileptic seizure focus, into the paraventricular hypothalamus to treat e.g., a satiety disorder, or into the amygdala central nucleus to treat e.g., a satiety disorder. In some embodiments, the recombinant nucleic acid may be administered to a subject by intraneural injection (i.e., directly into a nerve). The nerve may be selected based on the indication to be treated, for example, injection into the sciatic nerve to treat chronic pain or injection into the vagal nerve to treat epilepsy or a satiety disorder. In yet another particular case, a recombinant nucleic acid may be administered to a subject by injection, for example, into the sensory nerve terminals to treat chronic pain. In some embodiments, a recombinant nucleic acid of the disclosure is administered to the subject by direct injection into the epileptic focus in hippocampus.

Doses can vary and depend upon whether the treatment is prophylactic or therapeutic, the type, onset, progression, severity, frequency, duration, or probability of the disease treatment is directed to, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

In some embodiments, the recombinant nucleic acid is incorporated in a vector, such as a viral vector. In some embodiments, a vector dose may be expressed as the number of vector genome units delivered to a subject. The size of an individual vector genome will generally depend on the type of viral vector used. Vector genomes of the disclosure may be from about 1.0 kilobase, 1.5 kilobases, 2.0 kilobases, 2.5 kilobases, 3.0 kilobases, 3.5 kilobases, 4.0 kilobases, 4.5 kilobases, to 5.0 kilobases, or more than 5.0 kilobases, including all ranges and subranges therebetween.

In some embodiments, a vector dose of the administration has about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹, about 2×10⁹, about 3×10⁹, about 4×10⁹, about 5×10⁹, about 6×10⁹, about 7×10⁹, about 8×10⁹, about 9×10⁹, about 1×10¹⁰, about 2×10¹⁰, about 3×10¹⁰, about 4×10¹⁰, about 5×10¹⁰, about 6×10¹⁰, about 7×10¹⁰, about 8×10¹⁰, about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹, about 1×10¹², about 2×10¹², about 3×10¹², about 4×10¹², about 5×10¹², about 6×10¹², about 7×10¹², about 8×10¹², about 9×10¹², about 1×10¹³, about 2×10¹³, about 3×10¹³, about 4×10¹³, about 5×10¹³, about 6×10¹³, about 7×10¹³, about 8×10¹³, about 9×10¹³, about 1×10¹⁴, about 2×10¹⁴, about 3×10¹⁴, about 4×10¹⁴, about 5×10¹⁴, about 6×10¹⁴, about 7×10¹⁴, about 8×10¹⁴, about 9×10¹⁴, about 1×10¹⁵, about 2×10¹⁵, about 3×10¹⁵, about 4×10¹⁵, about 5×10¹⁵, about 6×10¹⁵, about 7×10¹⁵, about 8×10¹⁵, about 9×10¹⁵, about 1×10¹⁶, about 2×10¹⁶, about 3×10¹⁶, about 4×10¹⁶, about 5×10¹⁶, about 6×10¹⁶, about 7×10¹⁶, about 8×10¹⁶, about 9×10¹⁶, about 1×10¹⁷, or more vector genome units, including all ranges and subranges therebetween.

In some embodiments, a vector dose of the administration has at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 3×10⁸, at least 4×10⁸, at least 5×10⁸, at least 6×10⁸, at least 7×10⁸, at least 8×10⁸, at least 9×10⁸, at least 1×10⁹, at least 2×10⁹, at least 3×10⁹, at least 4×10⁹, at least 5×10⁹, at least 6×10⁹, at least 7×10⁹, at least 8×10⁹, at least 9×10⁹, at least 1×10¹⁰, at least 2×10¹⁰, at least 3×10¹⁰, at least 4×10¹⁰, at least 5×10¹⁰, at least 6×10¹⁰, at least 7×10¹⁰, at least 8×10¹⁰, at least 9×10¹⁰, at least 1×10¹¹, at least 2×10¹¹, at least 3×10¹¹, at least 4×10¹¹, at least 5×10¹¹, at least 6×10¹¹, at least 7×10¹¹, at least 8×10¹¹, at least 9×10¹¹, at least 1×10¹², at least 2×10¹², at least 3×10¹², at least 4×10¹², at least 5×10¹², at least 6×10¹², at least 7×10¹², at least 8×10¹², at least 9×10¹², at least 1×10¹³, at least 2×10¹³, at least 3×10¹³, at least 4×10¹³, at least 5×10¹³, at least 6×10¹³, at least 7×10¹³, at least 8×10¹³, at least 9×10¹³, at least 1×10¹⁴, at least 2×10¹⁴, at least 3×10¹⁴, at least 4×10¹⁴, at least 5×10¹⁴, at least 6×10¹⁴, at least 7×10¹⁴, at least 8×10¹⁴, at least 9×10¹⁴, at least 1×10¹⁵, at least 2×10¹⁵, at least 3×10¹⁵, at least 4×10¹⁵, at least 5×10¹⁵, at least 6×10¹⁵, at least 7×10¹⁵, at least 8×10¹⁵, at least 9×10¹⁵, at least 1×10¹⁶, at least 2×10¹⁶, at least 3×10¹⁶, at least 4×10¹⁶, at least 5×10¹⁶, at least 6×10¹⁶, at least 7×10¹⁶, at least 8×10¹⁶, at least 9×10¹⁶, or at least 1×10¹⁷ vector genome units, including all ranges and subranges therebetween.

In some embodiments, a vector dose of the administration has no more than 1×10⁶, no more than 2×10⁶, no more than 3×10⁶, no more than 4×10⁶, no more than 5×10⁶, no more than 6×10⁶, no more than 7×10⁶, no more than 8×10⁶, no more than 9×10⁶, no more than 1×10⁷, no more than 2×10⁷, no more than 3×10⁷, no more than 4×10⁷, no more than 5×10⁷, no more than 6×10⁷, no more than 7×10⁷, no more than 8×10⁷, no more than 9×10⁷, no more than 1×10⁸, no more than 2×10⁸, no more than 3×10⁸, no more than 4×10⁸, no more than 5×10⁸, no more than 6×10⁸, no more than 7×10⁸, no more than 8×10⁸, no more than 9×10⁸, no more than 1×10⁹, no more than 2×10⁹, no more than 3×10⁹, no more than 4×10⁹, no more than 5×10⁹, no more than 6×10⁹, no more than 7×10⁹, no more than 8×10⁹, no more than 9×10⁹, no more than 1×10¹⁰, no more than 2×10¹⁰, no more than 3×10¹⁰, no more than 4×10¹⁰, no more than 5×10¹⁰, no more than 6×10¹⁰, no more than 7×10¹⁰, no more than 8×10¹⁰, no more than 9×10¹⁰, no more than 1×10¹¹, no more than 2×10¹¹, no more than 3×10¹¹, no more than 4×10¹¹, no more than 5×10¹¹, no more than 6×10¹¹, no more than 7×10¹¹, no more than 8×10¹¹, no more than 9×10¹¹, no more than 1×10¹², no more than 2×10¹², no more than 3×10¹², no more than 4×10¹², no more than 5×10¹², no more than 6×10¹², no more than 7×10¹², no more than 8×10¹², no more than 9×10¹², no more than 1×10¹³, no more than 2×10¹³, no more than 3×10¹³, no more than 4×10¹³, no more than 5×10¹³, no more than 6×10¹³, no more than 7×10¹³, no more than 8×10¹³, no more than 9×10¹³, no more than 1×10¹⁴, no more than 2×10¹⁴, no more than 3×10¹⁴, no more than 4×10¹⁴, no more than 5×10¹⁴, no more than 6×10¹⁴, no more than 7×10¹⁴, no more than 8×10¹⁴, no more than 9×10¹⁴, no more than 1×10¹⁵, no more than 2×10¹⁵, no more than 3×10¹⁵, no more than 4×10¹⁵, no more than 5×10¹⁵, no more than 6×10¹⁵, no more than 7×10¹⁵, no more than 8×10¹⁵, no more than 9×10¹⁵, no more than 1×10¹⁶, no more than 2×10¹⁶, no more than 3×10¹⁶, no more than 4×10¹⁶, no more than 5×10¹⁶, no more than 6×10¹⁶, no more than 7×10¹⁶, no more than 8×10¹⁶, no more than 9×10¹⁶, or no more than 1×10¹⁷ vector genome units, including all ranges and subranges therebetween.

In some embodiments, a vector dose is expressed as vector genome units per kilogram of the weight of the subject (vg/kg). In some embodiments, the vector dose numbers described above are based on a subject of 50 kg in weight, and the dose can be translated to vg/kg accordingly and applied to another subject based on the weight of the subject. For example, a dose of about 5×10¹⁴ vector genome units for a subject of 50 kg in weight can be translated to about 1×10¹³ vector genome units per kilogram (vg/kg).

In some embodiments, a vector dose is expressed according to the concentration or titer of vector administered to a subject. In some embodiments, a vector dose may be expressed as the number of units per volume (e.g., genome units/volume) times the volume.

In some embodiments, a vector of the disclosure is administered in a volume of fluid. In some embodiments, the vector is administered in a volume of about 0.01 mL, about 0.02 mL, about 0.03 mL, about 0.04 mL, about 0.05 mL, about 0.06 mL, about 0.07 mL, about 0.08 mL, about 0.09 mL, about 0.1 mL, about 0.15 mL, about 0.2 mL, about 0.25 mL, about 0.3 mL, about 0.4 mL, about 0.5 mL, about 0.6 mL, about 0.7 mL, about 0.8 mL, about 0.9 mL, about 1.0 mL, about 2.0 mL, about 3.0 mL, about 4.0 mL, about 5.0 mL, about 6.0 mL, about 7.0 mL, about 8.0 mL, about 9.0 mL, about 10.0 mL, about 11.0 mL, about 12.0 mL, about 13.0 mL, about 14.0 mL, about 15.0 mL, about 16.0 mL, about 17.0 mL, about 18.0 mL, about 19.0 mL, about 20.0 mL, about 25.0 mL, or greater than 25.0 mL, including all ranges and subranges therebetween. In some embodiments, the vector is administered in a volume of at least 0.0 mL, at least 0.02 mL, at least 0.03 mL, at least 0.04 mL, at least 0.05 mL, at least 0.06 mL, at least 0.07 mL, at least 0.08 mL, at least 0.09 mL, at least 0.1 mL, at least 0.15 mL, at least 0.2 mL, at least 0.25 mL, at least 0.3 mL, at least 0.4 mL, at least 0.5 mL, at least 0.6 mL, at least 0.7 mL, at least 0.8 mL, at least 0.9 mL, at least 1.0 mL, at least 2.0 mL, at least 3.0 mL, at least 4.0 mL, at least 5.0 mL, at least 6.0 mL, at least 7.0 mL, at least 8.0 mL, at least 9.0 mL, at least 10.0 mL, at least 11.0 mL, at least 12.0 mL, at least 13.0 mL, at least 14.0 mL, at least 15.0 mL, at least 16.0 mL, at least 17.0 mL, at least 18.0 mL, at least 19.0 mL, at least 20.0 mL, or at least 25.0 mL, including all ranges and subranges therebetween. In some embodiments, the vector is administered in a volume of no more than 0.01 mL, no more than 0.02 mL, no more than 0.03 mL, no more than 0.04 mL, no more than 0.05 mL, no more than 0.06 mL, no more than 0.07 mL, no more than 0.08 mL, no more than 0.09 mL, no more than 0.1 mL, no more than 0.15 mL, no more than 0.2 mL, no more than 0.25 mL, no more than 0.3 mL, no more than 0.4 mL, no more than 0.5 mL, no more than 0.6 mL, no more than 0.7 mL, no more than 0.8 mL, no more than 0.9 mL, no more than 1.0 mL, no more than 2.0 mL, no more than 3.0 mL, no more than 4.0 mL, no more than 5.0 mL, no more than 6.0 mL, no more than 7.0 mL, no more than 8.0 mL, no more than 9.0 mL, no more than 10.0 mL, no more than 11.0 mL, no more than 12.0 mL, no more than 13.0 mL, no more than 14.0 mL, no more than 15.0 mL, no more than 16.0 mL, no more than 17.0 mL, no more than 18.0 mL, no more than 19.0 mL, no more than 20.0 mL, or no more than 25.0 mL including all ranges and subranges therebetween.

In some embodiments, a vector contemplated herein is administered to a subject at a titer of at least 1×10⁹ genome units/mL, at least 1×10¹⁰ genome units/mL, at least 5×10¹⁰ genome units/mL, at least 1×10¹¹ genome units/mL, at least 5×10¹¹ genome units/mL, at least 1×10¹² genome units/mL, at least 5×10¹² genome units/mL, at least 6×10¹² genome units/mL, at least 7×10¹² genome units/mL, at least 8×10¹² genome units/mL, at least 9×10¹² genome units/mL, at least 10×10¹² genome units/mL, at least 15×10¹² genome units/mL, at least 20×10¹² genome units/mL, at least 25×10¹² genome units/mL, at least 50×10¹² genome units/mL, or at least 100×10¹² genome units/mL, including all ranges and subranges therebetween. In some embodiments, a vector of the disclosure is administered to a subject at a titer of about 1×10⁹ genome units/mL, about 1×10¹⁰ genome units/mL, about 5×10¹⁰ genome units/mL, about 1×10¹¹ genome units/mL, about 5×10¹¹ genome units/mL, about 1×10¹² genome units/mL, about 5×10¹² genome units/mL, about 6×10¹² genome units/mL, about 7×10¹² genome units/mL, about 8×10¹² genome units/mL, about 9×10¹² genome units/mL, about 10×10¹² genome units/mL, about 15×10¹² genome units/mL, about 20×10¹² genome units/mL, about 25×10¹² genome units/mL, about 50×10¹² genome units/mL, or about 100×10¹² genome units/mL, including all ranges and subranges therebetween. In some embodiments, a vector of the disclosure is administered to a subject at a titer of no more than 1×10⁹ genome units/mL, no more than 1×10¹⁰ genome units/mL, no more than 5×10¹⁰ genome units/mL, no more than 1×10¹¹ genome units/mL, no more than 5×10¹¹ genome units/mL, no more than 1×10¹² genome units/mL, no more than 5×10¹² genome units/mL, no more than 6×10¹² genome units/mL, no more than 7×10¹² genome units/mL, no more than 8×10¹² genome units/mL, no more than 9×10¹² genome units/mL, no more than 10×10¹² genome units/mL, no more than 15×10¹² genome units/mL, no more than 20×10¹² genome units/mL, no more than 25×10¹² genome units/mL, no more than 50×10¹² genome units/mL, or no more than 100×10¹² genome units/mL, including all ranges and subranges therebetween.

In some embodiments, a vector contemplated herein is administered to a subject at a titer of about 5×10⁹ infectious units/mL, about 6×10⁹ infectious units/mL, about 7×10⁹ infectious units/mL, about 8×10⁹ infectious units/mL, about 9×10⁹ infectious units/mL, about 1×10¹⁰ infectious units/mL, about 1.5×10¹⁰ infectious units/mL, about 2×10¹⁰ infectious units/mL, about 2.5×10¹⁰ infectious units/mL, about 5×10¹⁰ infectious units/mL, about 1×10¹¹ infectious units/mL, about 2.5×10¹¹ infectious units/mL, about 5×10¹¹ infectious units/mL, about 1×10¹² infectious units/mL, about 2.5×10¹² infectious units/mL, about 5×10¹² infectious units/mL, about 1×10¹³ infectious units/mL, about 5×10¹³ infectious units/mL, or about 1×10¹⁴ infectious units/mL, including all ranges and subranges therebetween. In some embodiments, a vector contemplated herein is administered to a subject at a titer of at least 5×10⁹ infectious units/mL, at least 6×10⁹ infectious units/mL, at least 7×10⁹ infectious units/mL, at least 8×10⁹ infectious units/mL, at least 9×10⁹ infectious units/mL, at least 1×10¹⁰ infectious units/mL, at least 1.5×10¹⁰ infectious units/mL, at least 2×10¹⁰ infectious units/mL, at least 2.5×10¹⁰ infectious units/mL, at least 5×10¹⁰ infectious units/mL, at least 1×10¹¹ infectious units/mL, at least 2.5×10¹¹ infectious units/mL, at least 5×10¹¹ infectious units/mL, at least 1×10¹² infectious units/mL, at least 2.5×10¹² infectious units/mL, at least 5×10¹² infectious units/mL, at least 1×10¹³ infectious units/mL, at least 5×10¹³ infectious units/mL, or at least 1×10¹⁴ infectious units/mL, including all ranges and subranges therebetween. In some embodiments, a vector contemplated herein is administered to a subject at a titer of no more than 5×10⁹ infectious units/mL, no more than 6×10⁹ infectious units/mL, no more than 7×10⁹ infectious units/mL, no more than 8×10⁹ infectious units/mL, no more than 9×10⁹ infectious units/mL, no more than 1×10¹⁰ infectious units/mL, no more than 1.5×10¹⁰ infectious units/mL, no more than 2×10¹⁰ infectious units/mL, no more than 2.5×10¹⁰ infectious units/mL, no more than 5×10¹⁰ infectious units/mL, no more than 1×10¹¹ infectious units/mL, no more than 2.5×10¹¹ infectious units/mL, no more than 5×10¹¹ infectious units/mL, no more than 1×10¹² infectious units/mL, no more than 2.5×10¹² infectious units/mL, no more than 5×10¹² infectious units/mL, no more than 1×10¹³ infectious units/mL, no more than 5×10¹³ infectious units/mL, or no more than 1×10¹⁴ infectious units/mL, including all ranges and subranges therebetween.

In some embodiments, a vector of the disclosure is administered to a subject at a titer of about 5×10¹⁰ transducing units/mL, about 1×10¹¹ transducing units/mL, about 2.5×10¹¹ transducing units/mL, about 5×10¹¹ transducing units/mL, about 1×10¹² transducing units/mL, about 2.5×10¹² transducing units/mL, about 5×10¹² transducing units/mL, about 1×10¹³ transducing units/mL, about 5×10¹³ transducing units/mL, or about 1×10¹⁴ transducing units/mL, including all ranges and subranges therebetween. In some embodiments, a vector of the disclosure is administered to a subject at a titer of at least 5×10¹⁰ transducing units/mL, at least 1×10¹¹ transducing units/mL, at least 2.5×10¹¹ transducing units/mL, at least 5×10¹¹ transducing units/mL, at least 1×10¹² transducing units/mL, at least 2.5×10¹² transducing units/mL, at least 5×10¹² transducing units/mL, at least 1×10¹³ transducing units/mL, at least 5×10¹³ transducing units/mL, or at least 1×10¹⁴ transducing units/mL, including all ranges and subranges therebetween. In some embodiments, a vector of the disclosure is administered to a subject at a titer of no more than 5×10¹⁰ transducing units/mL, no more than 1×10¹¹ transducing units/mL, no more than 2.5×10¹¹ transducing units/mL, no more than 5×10¹¹ transducing units/mL, no more than 1×10¹² transducing units/mL, no more than 2.5×10¹² transducing units/mL, no more than 5×10¹² transducing units/mL, no more than 1×10¹³ transducing units/mL, no more than 5×10¹³ transducing units/mL, or no more than 1×10¹⁴ transducing units/mL, including all ranges and subranges therebetween.

In some embodiments, the vector dose is determined by the route of administration. In some embodiments, an intraganglionic injection may include from about 1×10⁹ to about 1×10¹³ vector genomes in a volume from about 0.1 mL to about 1.0 mL. In some embodiments, an intrathecal injection may include from about 1×10¹⁰ to about 1×10¹⁵ vector genomes in a volume from about 1.0 mL to about 12.0 mL. In some embodiments, an intracranial injection may include from about 1×10⁹ to about 1×10¹³ vector genomes in a volume from about 0.1 mL to about 1.0 mL. In some embodiments, an intraneural injection may include from about 1×10⁹ to about 1×10¹³ vector genomes in a volume from about 0.1 mL to about 1.0 mL. In some embodiments, an intraspinal injection may include from about 1×10⁹ to about 1×10¹³ vector genomes in a volume from about 0.1 mL to about 1.0 mL. In some embodiments, a Cisterna magna infusion may include from about 5×10⁹ to about 5×10¹³ vector genomes in a volume from about 0.5 mL to about 5.0 mL. In some embodiments, a subcutaneous injection may include from about 1×10⁹ to about 1×10¹³ vector genomes in a volume from about 0.1 mL to about 1.0 mL.

In some embodiments, the vector dose is the total dose of a single administration. In some embodiments, the vector dose is the total dose over a time period. In some embodiments, the time period is about 3 hours, about 6 hours, about 12 hours, about 1 day, about 2 days, about 3 days, about 4 days, about 5 days, about 6 days, about a week, about 2 weeks, about 3 weeks, about 4 weeks, about 1 months, about 2 months, about 3 months, about 4 months, about 5 months, about 6 months, about 12 months, or longer than 12 months. In some embodiments, the time period is within 3 hours, within 6 hours, within 12 hours, within 1 day, within 2 days, within 3 days, within 4 days, within 5 days, within 6 days, within a week, within 2 weeks, within 3 weeks, within 4 weeks, within 1 months, within 2 months, within 3 months, within 4 months, within 5 months, within 6 months, or within 12 months.

In some embodiments, the recombinant nucleic acid of the disclosure is delivered to a subject by infusion. A dose delivered to a subject by infusion can be measured as an infusion rate. Non-limiting examples of infusion rates include: 1-10 μL/min for intraganglionic, intraspinal, intracranial or intraneural administration; and 10-1000 μL/min for intrathecal or Cisterna magna administration. In some cases, the recombinant nucleic acid is delivered to a subject by MRI-guided Convection Enhanced Delivery (CED). This technique enables increased viral spread and transduction distributed throughout large volumes of the brain, as well as reduces reflux of the vector and/or nucleic acid along the needle path.

The present disclosure also provides dosing regiments for administering a ligand (e.g., small molecule LGIC agonist) in the cases which the administered recombinant nucleic acid encodes an engineered LGIC.

A therapeutically effective amount of a ligand can be administered once or more than once each day or over a longer period of time. In some cases, a therapeutically effective amount of a ligand is administered as needed (e.g., when pain relief or control of epilepsy is needed). The ligand may be administered serially (e.g., every day without a break for the duration of the treatment regimen). In some cases, the treatment regimen can be less than a week, a week, two weeks, three weeks, a month, or greater than a month. In some cases, a therapeutically effective amount of a ligand is administered for a day, at least two consecutive days, at least three consecutive days, at least four consecutive days, at least five consecutive days, at least six consecutive days, at least seven consecutive days, at least eight consecutive days, at least nine consecutive days, at least ten consecutive days, or at least greater than ten consecutive days. In a particular case, a therapeutically effective amount of a ligand is administered for three consecutive days. In some cases, a therapeutically effective amount of a ligand can be administered one time per week, two times per week, three times per week, four times per week, five times per week, six times per week, seven times per week, eight times per week, nine times per week, 10 times per week, 11 times per week, 12 times per week, 13 times per week, 14 times per week, 15 times per week, 16 times per week, 17 times per week, 18 times per week, 19 times per week, 20 times per week, 25 times per week, 30 times per week, 35 times per week, 40 times per week, or greater than 40 times per week. In some cases, a therapeutically effective amount of a ligand can be administered one time per day, two times per day, three times per day, four times per day, five times per day, six times per day, seven times per day, eight times per day, nine times per day, 10 times per day, or greater than 10 times per day. In some cases, a therapeutically effective amount of a ligand is administered at least every hour, at least every two hours, at least every three hours, at least every four hours, at least every five hours, at least every six hours, at least every seven hours, at least every eight hours, at least every nine hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, or at least every day. The dose of ligand may be administered to the subject continuously, or 1, 2, 3, 4, or 5 times a day; 1, 2, 3, 4, 5, 6, or 7 times a week, 1, 2, 3, or 4 times a month, once every 2, 3, 4, 5, or 6 months, or once a year, or at even longer intervals. The duration of treatment can last a day, 1, 2, or 3 weeks, 1, 2, 3, 4, 5, 7, 8, 9, 10, or 11 months, 1, 2, 3, 4, 5, or more years, or longer.

The number of times a composition is administered to an subject in need thereof depends on the discretion of a medical professional, the disorder, the severity of the disorder, and the subject's response to the formulation. In some embodiments, administration of a composition occurs at least once. In some embodiments, administration occurs more than once, for example 2, 3, 4, 5, 6, 7, 8, 9, 10 or more times in a given period. The dosage of each administration and/or frequency of administrations may be adjusted as necessary based on the patient's condition and physiologically responses.

### Methods for Generating an AAV Virion

In one aspect, the disclosure provides methods for generating an AAV virion of the disclosure. Generally, the methods involve inserting or transducing an AAV vector of the disclosure into a host cell capable of packaging the AAV vector into an AAV virion. Exemplary methods are described and referenced below; however, any method known to one of skill in the art can be employed to generate the AAV virions of the disclosure.

An AAV vector comprising a heterologous nucleic acid and used to generate an AAV virion can be constructed using methods that are well known in the art. See, e.g., Koerber et al. (2009) Mol. Ther., 17:2088; Koerber et al. (2008) Mol Ther., 16: 1703-1709; as well as U.S. Pat. Nos. 7,439,065, 6,951,758, and 6,491,907. For example, the heterologous sequence(s) can be directly inserted into an AAV genome with the major AAV open reading frames (“ORFs”) excised therefrom. Other portions of the AAV genome can also be deleted, so long as a sufficient portion of the ITRs remain to allow for replication and packaging functions.

In order to produce AAV virions, an AAV vector is introduced into a suitable host cell using known techniques, such as by transfection. Suitable host cells for producing AAV virions include any species and/or type of cell that can be, or have been, used as recipients of a heterologous AAV DNA molecule, and can support the expression of required AAV production cofactors from helper viruses. Such host cells can include but are not limited to microorganisms, yeast cells, insect cells, and mammalian cells, that can be, or have been, used as recipients of a heterologous DNA molecule. The term includes the progeny of the original cell transfected. Thus, a “host cell” as used herein generally refers to a cell transfected with an exogenous DNA sequence. Cells from the stable human cell line, HEK293 (readily available through, e.g., the American Type Culture Collection under Accession Number ATCC CRL1573) can be used.

Methods of producing an AAV virion in insect cells are known in the art, and can be used to produce a subject AAV virion. See, e.g., U.S. Patent Publication No. 2009/0203071; U.S. Pat. No. 7,271,002; and Chen (2008) Mol. Ther. 16:924.

### Kit

In one aspect, the present disclosure provides kits comprising a recombinant nucleic acid, wherein the recombinant nucleic acid comprises the expression cassette of the disclosure. Ins some embodiments, the expression cassette of the recombinant nucleic acid comprises a transgene encoding an engineered receptor of the disclosure. In some embodiments, the recombinant nucleic acid is incorporated in a vector. In some embodiments, the vector is a viral vector. In some embodiments, the vector is an AAV vector. some embodiments, the kit comprises a non-native ligand of the disclosure.

In some embodiments, the kit comprises (a) the recombinant nucleic acid or the vector comprising the recombinant nucleic acid; and (b) instructions for administering the recombinant nucleic acid or vector to a dorsal root ganglion neuron or a trigeminal ganglion neuron. In some embodiments, the kit comprises device adapted for intrathecal (IT) or intraganglionic (IG) administration.

In some embodiments, the kit comprises (a) the recombinant nucleic acid or the vector comprising the recombinant nucleic acid; and (b) instructions for administering the recombinant nucleic acid or vector to a hippocampal neuron. In some embodiments, the kit comprises a device adapted for intracranial administration.

In some embodiments, the kit further comprises packaging material and one or more components therein. A kit typically includes a label or packaging insert including a description of the components or instructions for use in vitro, in vivo, or ex vivo, of the components therein. A kit can contain a collection of such components, e.g., the recombinant nucleic acid or the corresponding vector, and optionally a second active component, such as another compound, agent, drug or composition. In some embodiments, the recombinant nucleic acid encodes an LGIC (e.g., engineered LGIC) and the second active agent is a ligand (e.g., small molecule drug) for the LGIC.

A kit refers to a physical structure housing one or more components of the kit. Packaging material can maintain the components sterilely and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying the manufacturer, lot numbers, manufacturer location and date, expiration dates. Labels or inserts can include information identifying manufacturer information, lot numbers, manufacturer location and date. Labels or inserts can include information on a disease a kit component may be used for. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another incompatible treatment protocol or therapeutic regimen and, therefore, instructions could include information regarding such incompatibilities.

### FURTHER NUMBERED EMBODIMENTS

Further numbered embodiments of the present disclosure are provided as follows:

Embodiment 1. A recombinant nucleic acid comprising an expression cassette comprising, in 5′ to 3′ order, one or more of a 5′ enhancer, a promoter, a 5′ untranslated region (UTR), a transgene, a 3′ enhancer, and a polyadenylation sequence (polyA), wherein the transgene is operably linked to the promoter.

Embodiment 2. The recombinant nucleic acid of Embodiment 1, wherein the expression cassette comprises the 5′ enhancer comprising a polynucleotide sequence at least 90% identical to any one of SEQ ID NO: 37-39.

Embodiment 3. The recombinant nucleic acid of Embodiment 1, wherein the expression cassette does not comprise the 5′ enhancer.

Embodiment 4. The recombinant nucleic acid of any one of Embodiments 1-3, wherein the expression cassette comprises the promoter comprising a polynucleotide sequence at least 90% identical to any one of SEQ ID NO: 41-51.

Embodiment 5. The recombinant nucleic acid of any one of Embodiments 1-4, wherein the promoter is a neuron-specific promoter.

Embodiment 6. The recombinant nucleic acid of any one of Embodiments 1-5, wherein the expression cassette comprises an intron between the promoter and the transgene.

Embodiment 7. The recombinant nucleic acid of any one of Embodiments 1-5, wherein the expression cassette comprises an intron between the 5′ UTR and the transgene.

Embodiment 8. The recombinant nucleic acid of any one of Embodiments 1-5, wherein the expression cassette comprises an intron between the promoter and the 5′ UTR.

Embodiment 9. The recombinant nucleic acid of any one of Embodiments 6-8, wherein the intron comprises a polynucleotide sequence at least 90% identical to any one of SEQ ID NO: 57-61.

Embodiment 10. The recombinant nucleic acid of any one of Embodiments 1-5, wherein the expression cassette does not comprise an intron.

Embodiment 11. The recombinant nucleic acid of any one of Embodiments 1-10, wherein the expression cassette comprises the 5′ UTR comprising a polynucleotide sequence at least 90% identical to any one of SEQ ID NO: 52-56.

Embodiment 12. The recombinant nucleic acid of any one of Embodiments 1-11, wherein the expression cassette comprises the 3′ enhancer comprising a polynucleotide sequence at least 90% identical to any one of SEQ ID NO: 62-65.

Embodiment 13. The recombinant nucleic acid of any one of Embodiments 1-12, wherein the expression cassette comprises the polyA comprising a polynucleotide sequence at least 90% identical to any one of SEQ ID NO: 67-70.

Embodiment 14. The recombinant nucleic acid of any one of Embodiments 1-13, wherein the expression cassette comprises a non-neuron silencing element embedded in the promoter, and wherein the non-neuron silencing element comprises a polynucleotide sequence at least 90% identical to SEQ ID NO: 40.

Embodiment 15. The recombinant nucleic acid of any one of Embodiments 1-13, wherein the expression cassette comprises a non-neuron silencing element between the 5′ enhancer and the promoter, and wherein the non-neuron silencing element comprises a polynucleotide sequence at least 90% identical to SEQ ID NO: 40.

Embodiment 16. The recombinant nucleic acid of any one of Embodiments 1-15, wherein the expression cassette comprises a 3′ UTR between the 3′ enhancer and the polyA, and wherein the 3′ UTR comprises a polynucleotide sequence at least 90% identical to SEQ ID NO: 66.

Embodiment 17. The recombinant nucleic acid of any one of Embodiments 1-16, wherein the transgene encodes a ligand-gated ion channel (LGIC).

Embodiment 18. The recombinant nucleic acid of Embodiments 17, wherein the ligand-gated ion channel comprises a ligand binding domain derived from human α7 nicotinic acetylcholine receptor (α7-nAChR).

Embodiment 19. The recombinant nucleic acid of Embodiment 18, wherein the ligand binding domain comprises an amino acid sequence having at least 85% identity to amino acid residues 23-220 of SEQ ID NO: 25.

Embodiment 20. The recombinant nucleic acid of Embodiment 18 or 19, wherein the ligand binding domain comprises one or more amino acid mutations listed in Table 5.

Embodiment 21. The recombinant nucleic acid of any one of Embodiments 18-20, wherein ligand-gated ion channel comprises an ion pore domain derived from a human Glycine receptor.

Embodiment 22. The recombinant nucleic acid of Embodiment 21, wherein the ion pore domain comprises an amino acid sequence having at least 85% identity to amino acids 255-457 of SEQ ID NO: 26, 260-452 of SEQ ID NO: 27, amino acids 259-464 of SEQ ID NO: 28, or amino acids 259-449 of SEQ ID NO: 29.

Embodiment 23. The recombinant nucleic acid of Embodiment 21 or 22, wherein the ligand binding domain of the engineered receptor comprises a Cys-loop domain derived from the human Glycine receptor.

Embodiment 24. The recombinant nucleic acid of any one of Embodiments 21-23, wherein the ligand-gated ion channel comprises an amino acid sequence having at least 95% sequence identity to any one of SEQ ID NO: 25-31 and 33.

Embodiment 25. The recombinant nucleic acid of any one of Embodiments 21-23, wherein the human Glycine receptor is human Glycine receptor α1, and wherein the ligand-gated ion channel comprises an amino acid sequence having at least 95% sequence identity to SEQ ID NO: 33.

Embodiment 26. The recombinant nucleic acid of any one of Embodiments 1-25, wherein the transgene comprises or consists of a polynucleotide sequence having at least 90%, at least 95%, at least 96%, at least 97%, at least 98%, at least 99%, at least 99.5%, or 100% identity to SEQ ID NO: 32.

Embodiment 27. The recombinant nucleic acid of any one of Embodiments 1-26, wherein the transgene is codon-optimized for expression in a human cell.

Embodiment 28. The recombinant nucleic acid of Embodiment 27, wherein the human cell is a neuron.

Embodiment 29. The recombinant nucleic acid of any one of Embodiments 1-28, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(iii) a hCaMKII 5′ UTR comprising a polynucleotide sequence having at least 90%, at least 95%, at least 96%, at least 97%, at least 98%, at least 99%, at least 99.5%, or 100% identity to SEQ ID NO: 53;
(iv) a hCMV+rGlob Intron comprising a polynucleotide sequence having at least 90%, at least 95%, at least 96%, at least 97%, at least 98%, at least 99%, at least 99.5%, or 100% identity to SEQ ID NO: 58;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68.

Embodiment 30. The recombinant nucleic acid of Embodiment 29, wherein the expression cassette comprises, in 5′ to 3′ order, a sequence having at least 90%, at least 95%, at least 96%, at least 97%, at least 98%, at least 99%, at least 99.5%, or 100% identity to nucleotides 173-1877 of SEQ ID NO: 108, the transgene, and a sequence having at least 90%, at least 95%, at least 96%, at least 97%, at least 98%, at least 99%, at least 99.5%, or 100% identity to nucleotides 3237-4315 of SEQ ID NO: 108.

Embodiment 31. The recombinant nucleic acid of any one of Embodiments 1-28, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hSyn intron comprising a polynucleotide sequence having at least 90%, at least 95%, at least 96%, at least 97%, at least 98%, at least 99%, at least 99.5%, or 100% identity to SEQ ID NO: 59;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68.

Embodiment 32. The recombinant nucleic acid of Embodiment 31, wherein the expression cassette comprises, in 5′ to 3′ order, a sequence having at least 90%, at least 95%, at least 96%, at least 97%, at least 98%, at least 99%, at least 99.5%, or 100% identity to nucleotides 173-1159 of SEQ ID NO: 106, the transgene, and a sequence having at least 90%, at least 95%, at least 96%, at least 97%, at least 98%, at least 99%, at least 99.5%, or 100% identity to nucleotides 2519-3597 of SEQ ID NO: 106.

Embodiment 33. The recombinant nucleic acid of any one of Embodiments 1-28, wherein the expression cassette comprises, in 5′ to 3′ order,
(i) a CMV-V2 5′ enhancer comprising a polynucleotide sequence having at least 90%, at least 95%, at least 96%, at least 97%, at least 98%, at least 99%, at least 99.5%, or 100% identity to SEQ ID NO: 38;
(ii) a hSyn-V2 promoter comprising a polynucleotide sequence having at least 90%, at least 95%, at least 96%, at least 97%, at least 98%, at least 99%, at least 99.5%, or 100% identity to SEQ ID NO: 49;
(iii) the transgene;
(iv) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(v) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

Embodiment 34. The recombinant nucleic acid of Embodiment 33, wherein the expression cassette comprises, in 5′ to 3′ order, a sequence having at least 90%, at least 95%, at least 96%, at least 97%, at least 98%, at least 99%, at least 99.5%, or 100% identity to nucleotides 149-946 of SEQ ID NO: 125, the transgene, and a sequence having at least 90%, at least 95%, at least 96%, at least 97%, at least 98%, at least 99%, at least 99.5%, or 100% identity to nucleotides 2285-3416 of SEQ ID NO: 125.

Embodiment 35. The recombinant nucleic acid of any one of Embodiments 1-28, wherein the expression cassette comprises, in 5′ to 3′ order,
(i) a CMV-V2 5′ enhancer comprising a polynucleotide sequence having at least 90%, at least 95%, at least 96%, at least 97%, at least 98%, at least 99%, at least 99.5%, or 100% identity to SEQ ID NO: 38;
(ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(iii) the transgene;
(iv) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(v) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

Embodiment 36. The recombinant nucleic acid of Embodiment 35, wherein the expression cassette comprises, in 5′ to 3′ order, a sequence having at least 90%, at least 95%, at least 96%, at least 97%, at least 98%, at least 99%, at least 99.5%, or 100% identity to nucleotides 149-1527 of SEQ ID NO: 126, the transgene, and a sequence having at least 90%, at least 95%, at least 96%, at least 97%, at least 98%, at least 99%, at least 99.5%, or 100% identity to nucleotides 2848-3979 of SEQ ID NO: 126.

Embodiment 37. The recombinant nucleic acid of any one of Embodiments 1-28, wherein the expression cassette comprises, in 5′ to 3′ order,
(i) a hSyn-V2 promoter comprising a polynucleotide sequence having at least 90%, at least 95%, at least 96%, at least 97%, at least 98%, at least 99%, at least 99.5%, or 100% identity to SEQ ID NO: 49;
(ii) the transgene;
(iii) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(iv) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

Embodiment 38. The recombinant nucleic acid of Embodiment 37, wherein the expression cassette comprises, in 5′ to 3′ order, a sequence having at least 90%, at least 95%, at least 96%, at least 97%, at least 98%, at least 99%, at least 99.5%, or 100% identity to nucleotides 153-661 of SEQ ID NO: 127, the transgene, and a sequence having at least 90%, at least 95%, at least 96%, at least 97%, at least 98%, at least 99%, at least 99.5%, or 100% identity to nucleotides 1982-3113 of SEQ ID NO: 127.

Embodiment 39. The recombinant nucleic acid of any one of Embodiments 1-28, wherein the expression cassette comprises, in 5′ to 3′ order,
(i) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(ii) the transgene;
(iii) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(iv) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

Embodiment 40. The recombinant nucleic acid of Embodiment 39, wherein the expression cassette comprises, in 5′ to 3′ order, a sequence having at least 90%, at least 95%, at least 96%, at least 97%, at least 98%, at least 99%, at least 99.5%, or 100% identity to nucleotides 153-1224 of SEQ ID NO: 128, the transgene, and a sequence having at least 90%, at least 95%, at least 96%, at least 97%, at least 98%, at least 99%, at least 99.5%, or 100% identity to nucleotides 2545-3676 of SEQ ID NO: 128.

Embodiment 41. The recombinant nucleic acid of any one of Embodiments 1-28, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hTPI intron comprising a polynucleotide sequence having at least 90%, at least 95%, at least 96%, at least 97%, at least 98%, at least 99%, at least 99.5%, or 100% identity to SEQ ID NO: 60; (v) the transgene;
(vi) a FullEES 3′ enhancer comprising a polynucleotide sequence having at least 90%, at least 95%, at least 96%, at least 97%, at least 98%, at least 99%, at least 99.5%, or 100% identity to SEQ ID NO: 63; and
(vii) a rβGlobin polyA comprising a polynucleotide sequence having at least 90%, at least 95%, at least 96%, at least 97%, at least 98%, at least 99%, at least 99.5%, or 100% identity to SEQ ID NO: 70.

Embodiment 42. The recombinant nucleic acid of Embodiment 41, wherein the expression cassette comprises, in 5′ to 3′ order, a sequence having at least 90%, at least 95%, at least 96%, at least 97%, at least 98%, at least 99%, at least 99.5%, or 100% identity to nucleotides 173-1188 of SEQ ID NO: 98, the transgene, and a sequence having at least 90%, at least 95%, at least 96%, at least 97%, at least 98%, at least 99%, at least 99.5%, or 100% identity to nucleotides 2548-3824 of SEQ ID NO: 98.

Embodiment 43. The recombinant nucleic acid of any one of Embodiments 1-42, wherein the expression cassette comprises (i) a polynucleotide sequence having at least 90% identity to any one of SEQ ID NO: 71-93 excluding the sequence of the transgene (SEQ ID NO: 36), or (ii) a polynucleotide sequence having at least 90% identity to any one of SEQ ID NO: 121-124 excluding the sequence of the transgene (SEQ ID NO: 32).

Embodiment 44. The recombinant nucleic acid of any one of Embodiments 1-42, wherein the expression cassette comprises a polynucleotide sequence having at least 90% identity to any one of SEQ ID NO: 71-93 and 121-124.

Embodiment 45. The recombinant nucleic acid of any one of Embodiments 1-44, comprising an adeno-associated virus (AAV) inverted terminal repeat (ITR) flanking each end of the expression cassette.

Embodiment 46. The recombinant nucleic acid of Embodiment 45, comprising a 5′ ITR sequence having at least 90% identity to SEQ ID NO: 94 or 119 and a 3′ ITR sequence having at least 90% identity to SEQ ID NO: 95 or 120.

Embodiment 47. The recombinant nucleic acid of any one of Embodiments 1-45, comprising (i) a polynucleotide sequence having at least 90% identity to any one of SEQ ID NO: 96-118 excluding the sequence of the transgene (SEQ ID NO: 36), or (ii) a polynucleotide sequence having at least 90% identity to any one of SEQ ID NO: 125-128 excluding the sequence of the transgene (SEQ ID NO: 32).

Embodiment 48. The recombinant nucleic acid of any one of Embodiments 1-45, comprising a polynucleotide sequence having at least 90% identity to any one of SEQ ID NO: 96-118 and 125-128.

Embodiment 49. A vector comprising the recombinant nucleic acid of any one of Embodiments 1-48.

Embodiment 50. The vector of Embodiment 49, wherein the vector is a non-viral vector.

Embodiment 51. The vector of Embodiment 49, wherein the vector is a viral vector.

Embodiment 52. The vector of Embodiment 51, wherein the vector comprises or consists of an AAV vector genome.

Embodiment 53. An AAV comprising the vector of Embodiments 51 or 52.

Embodiment 54. The AAV of Embodiment 53, wherein the AAV is AAV9 serotype.

Embodiment 55. The AAV of Embodiment 53 or 54, wherein the AAV is a self-complementary AAV or a single stranded AAV.

Embodiment 56. The AAV of any one of Embodiments 53-55, wherein the AAV is a wild-type AAV or a modified AAV.

Embodiment 57. The AAV of any one of Embodiments 53-56, wherein the AAV comprises a capsid protein having at least 95% identity to an AAV9 capsid protein (SEQ ID NO: 8) or AAV9-TV capsid protein (SEQ ID NO: 9).

Embodiment 58. A host cell, comprising the nucleic acid of any one of Embodiments 1-48, or the vector of any one of Embodiments 49-52.

Embodiment 59. A method of producing the AAV of any one of Embodiments 53-57.

Embodiment 60. A kit comprising the recombinant nucleic acid of any one of Embodiments 1-48, the vector of any one of Embodiments 49-52, or the AAV of any one of Embodiments 53-57.

Embodiment 61. A method of expressing a transgene in a cell, comprising delivering the recombinant nucleic acid of any one of Embodiments 1-48 or the vector of any one of Embodiments 49-52 to the cell.

Embodiment 62. A method of transducing a cell, comprises contacting the cell with the AAV of any one of Embodiments 53-57.

Embodiment 63. The method of Embodiment 61 or 62, wherein the cell is a neuron.

Embodiment 64. The method of Embodiment 63, wherein the neuron is a hippocampal neuron.

Embodiment 65. The method of Embodiment 64, wherein the neuron is an excitatory neuron.

Embodiment 66. The method of Embodiment 65, wherein the neuron is a CAMK2 positive neuron.

Embodiment 67. The method of Embodiment 64, wherein the neuron is an inhibitory neuron.

Embodiment 68. The method of Embodiment 67, wherein the neuron is a GABAergic neuron.

Embodiment 69. The method of Embodiment 63, wherein the neuron is a dorsal root ganglion neuron or a trigeminal ganglion neuron.

Embodiment 70. The method of Embodiment 69, wherein the neuron comprises an isolectin B4 (IB4) positive nerve fiber.

Embodiment 71. The method of Embodiment 69 or 70, wherein the neuron comprises an NF200 positive nerve fiber.

Embodiment 72. The method of any one of Embodiments 69-71, wherein the neuron comprises a CGRP positive nerve fiber.

Embodiment 73. The method of any one of Embodiments 69-72, wherein the neuron comprises a C fiber.

Embodiment 74. The method of any one of Embodiments 69-73, wherein the neuron comprises an Aδ fiber.

Embodiment 75. The method of any one of Embodiments 61-74, wherein the cell is an ex vivo cell.

Embodiment 76. The method of any one of Embodiments 61-74, wherein the cell is an in vivo cell of a subject, optionally wherein the subject is a human.

Embodiment 77. The method of any one of Embodiments 61-76, wherein the cell comprising the expression cassette has a higher expression level of the transgene compared to a corresponding cell comprising a control expression cassette, optionally wherein said higher expression is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least ten-fold higher than the transgene expression level of the control expression cassette comprising the polynucleotide sequence of SEQ ID NO: 88 excluding the transgene sequence.

Embodiment 78. The method of any one of Embodiments 61-76, wherein the cell comprising the expression cassette has a comparable or higher expression level of the transgene compared to a corresponding cell comprising a control expression cassette, optionally wherein said transgene expression level is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least ten-fold of the transgene expression level of the control expression cassette comprising the polynucleotide sequence of SEQ ID NO: 87 excluding the transgene sequence.

Embodiment 79. A method of treating a disease or disorder in a subject in need thereof, comprises administering an effective amount of the recombinant nucleic acid of any one of Embodiments 1-48, the vector of any one of Embodiments 49-52, or the AAV of any one of Embodiments 53-57 to the subject.

Embodiment 80. The method of Embodiment 79, wherein the disease or disorder is epilepsy, schizophrenia, autism spectrum disorder, Alzheimer's disease, Rett syndrome, or fragile X syndrome.

Embodiment 81. The method of Embodiment 79, wherein the disease or disorder is focal epilepsy.

Embodiment 82. The method of Embodiment 79, wherein the disease or disorder is mesial temporal lobe epilepsy (mTLE).

Embodiment 83. The method of any one of Embodiments 61-82, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(iii) a hCaMKII 5′ UTR comprising a polynucleotide sequence having at least 90%, at least 95%, at least 96%, at least 97%, at least 98%, at least 99%, at least 99.5%, or 100% identity to SEQ ID NO: 53;
(iv) a hCMV+rGlob Intron comprising a polynucleotide sequence having at least 90%, at least 95%, at least 96%, at least 97%, at least 98%, at least 99%, at least 99.5%, or 100% identity to SEQ ID NO: 58;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68, optionally, wherein the transgene encodes a ligand-gated ion channel comprising a ligand binding domain derived from human α7-nAChR and an ion pore domain derived from a human Glycine receptor.

Embodiment 84. The method of any one of Embodiments 61-82, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hSyn intron comprising a polynucleotide sequence having at least 90%, at least 95%, at least 96%, at least 97%, at least 98%, at least 99%, at least 99.5%, or 100% identity to SEQ ID NO: 59;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68, optionally, the transgene encodes a ligand-gated ion channel comprising a ligand binding domain derived from human α7-nAChR and an ion pore domain derived from a human Glycine receptor.

Embodiment 85. The method of any one of Embodiments 79-84, wherein the recombinant nucleic acid, the vector, or the AAV is administered by intracranial administration, intrathecal (spine) administration, intrathecal (Cisterna magna) administration, intracerebral administration, intraventricular administration, or direct injection into the epileptic focus in hippocampus.

Embodiment 86. The method of any one of Embodiments 79-84, wherein the recombinant nucleic acid, the vector, or the AAV is administered by direct injection into the epileptic focus in hippocampus.

Embodiment 87. The method of any one of Embodiments 79-86, wherein 1×10⁹-1×1014 copies of the recombinant nucleic acid or AAV vector genome are administered to the subject.

Embodiment 88. The method of any one of Embodiments 79-87, wherein the AAV comprises a capsid protein of AAV9 (SEQ ID NO: 8).

Embodiment 89. The method of any one of Embodiments 79-88, wherein the method decreases the duration, intensity, and/or frequency of the epilepsy by at least 10%.

Embodiment 90. A method of treating a disease or disorder in a subject in need thereof, comprises administering an effective amount of the recombinant nucleic acid of any one of Embodiments 1-48, the vector of any one of Embodiments 49-52, or the AAV of any one of Embodiments 53-57 to the subject, wherein the disease or disorder is neuropathic pain, spasticity, spinal cord injury, or avulsion injury.

Embodiment 91. The method of Embodiment 90, wherein the disease or disorder is neuropathic pain.

Embodiment 92. The method of Embodiment 91, wherein the neuropathic pain is peripheral neuropathy.

Embodiment 93. The method of Embodiment 91, wherein the neuropathic pain is trigeminal neuralgia.

Embodiment 94. The method of any one of Embodiments 61-78 and 90-93, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hTPI intron comprising a polynucleotide sequence having at least 90%, at least 95%, at least 96%, at least 97%, at least 98%, at least 99%, at least 99.5%, or 100% identity to SEQ ID NO: 60;
(v) the transgene;
(vi) a FullEES 3′ enhancer comprising a polynucleotide sequence having at least 90%, at least 95%, at least 96%, at least 97%, at least 98%, at least 99%, at least 99.5%, or 100% identity to SEQ ID NO: 63; and
(vii) a rβGlobin polyA comprising a polynucleotide sequence having at least 90%, at least 95%, at least 96%, at least 97%, at least 98%, at least 99%, at least 99.5%, or 100% identity to SEQ ID NO: 70, optionally, the transgene encodes a ligand-gated ion channel comprising a ligand binding domain derived from human α7-nAChR and an ion pore domain derived from a human Glycine receptor.

Embodiment 95. The method of any one of Embodiments 61-78 and 90-93, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hSyn intron comprising a polynucleotide sequence having at least 90%, at least 95%, at least 96%, at least 97%, at least 98%, at least 99%, at least 99.5%, or 100% identity to SEQ ID NO: 59;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68, optionally, the transgene encodes a ligand-gated ion channel comprising a ligand binding domain derived from human α7-nAChR and an ion pore domain derived from a human Glycine receptor.

Embodiment 96. The method of any one of Embodiments 90-95, wherein the recombinant nucleic acid, the vector, or the AAV is administered by intrathecal (IT) or intraganglionic (IG) administration.

Embodiment 97. The method of any one of Embodiments 90-95, wherein the recombinant nucleic acid, the vector, or the AAV is administered by intraganglionic (IG) administration directly into dorsal root ganglion or trigeminal ganglion.

Embodiment 98. The method of any one of Embodiments 90-97, wherein 1×10⁹-1×1014 copies of the recombinant nucleic acid or AAV vector genome are administered to the subject.

Embodiment 99. The method of any one of Embodiments 90-98, wherein the AAV comprises a capsid protein of AAV9-TV (SEQ ID NO: 9).

Embodiment 100. The method of any one of Embodiments 91-99, wherein the method lowers the level of the pain by at least 10%.

Embodiment 101. The method of any one of Embodiments 79-100, wherein the recombinant nucleic acid, the vector, or the AAV is administered by systemic, parenteral, intravenous, cerebral, cerebrospinal, intrathecal, intracisternal, intraputaminal, intrahippocampal, intra-striatal, or intra-cerebroventricular injection.

Embodiment 102. The method of any one of Embodiments 79-101, wherein 1×109-1×10¹⁴ copies of the recombinant nucleic acid or AAV vector genome are administered to the subject.

Embodiment 103. The method of any one of Embodiments 79-102, wherein the method comprises administering a ligand of a ligand-gated ion channel encoded by the transgene.

Embodiment 104. The method of Embodiment 103, wherein the ligand is selected from the group consisting of AZD-0328, TC-6987, ABT-126, TC-5619, TC-6683, Varenicline, and Facinicline/RG3487.

### EXAMPLES

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Example 1: In Vitro Assessment of Expression Cassettes with Optimal Hippocampal Neuron Expression for the Treatment of Focal Epilepsy

Expression cassettes: Twenty-three expression cassettes with ITRs flanking different combinations of regulatory elements were generated by DNA synthesis and using usual recombinant DNA cloning techniques.

Culture and transfection of SK-N-AS & HeLa cells: Cells were thawed and plated in complete growth medium. Cells were plated at 2E4 cells per well of a 96-well plate the day before transfection and subsequently transfected with 23 different expression cassettes in duplicate using FuGENE HD reagent at a ratio of 3:1 (FuGENE (mL): DNA (mg)).

Culture and transduction of neonatal rat hippocampal mixed cultures: Embryonic hippocampal tissue was extracted from an 18-day pregnant female rat, dissociated and plated in appropriate cell culture media. Cells were transduced with AAV9-carrying different expression cassettes at an MOI of 3E5 and processed 3 to 5 days later for mRNA or protein analysis.

ddPCR assessment: RNA was isolated from cells for RNeasy Plus Mini Kit and PureLink™ DNase, using the manufacturer's protocol. cDNA was synthesized using SuperScript™ IV VILO™ Master Mix with ezDNase™ Enzyme kit. ddPCR reactions were carried out with customs primers purchased from Integrated DNA Technologies, TaqMan™ probes from Thermo Fisher Scientific, and the rest of the reagents and instruments from Bio-Rad. Expression of the receptor transgene was compared to a house-keeping gene in the same reaction and presented as a ratio of the two.

ELISA assessment: Cell culture samples were processed using a DYKDDDDK-Tag Protein ELISA Kit according to the manufacturer's protocol. Total protein concentrations were determined per sample using a Micro BCA™ Protein Assay Kit following the manufacture's protocol.

Description of Experiments: A series of AAV based expression cassettes were generated and the expression of the encoded ligand gated ion channel (LGIC) was tested in target cells of the hippocampus. The LGIC is a FLAG-tagged CODA receptor having the polynucleotide sequence of SEQ ID NO: 36.

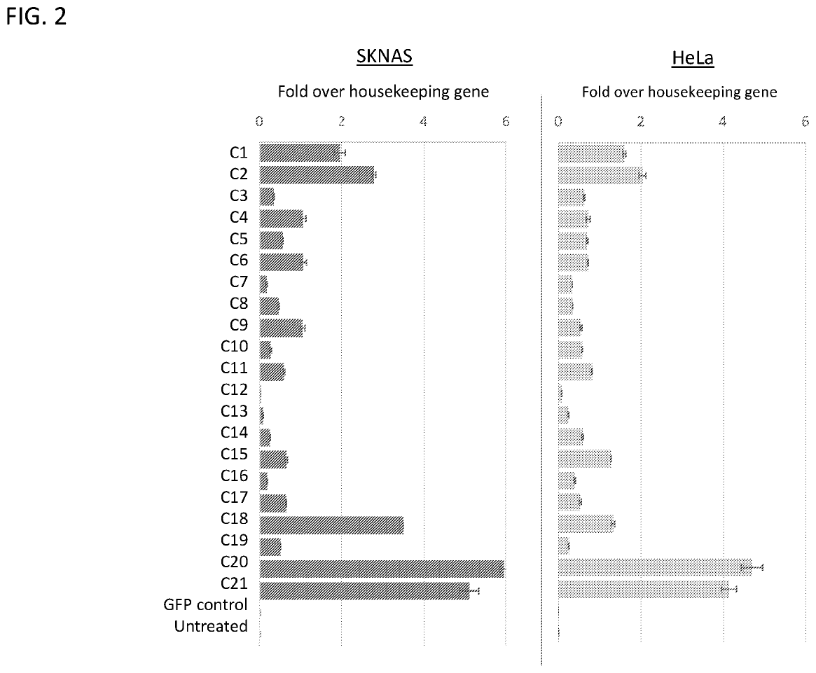

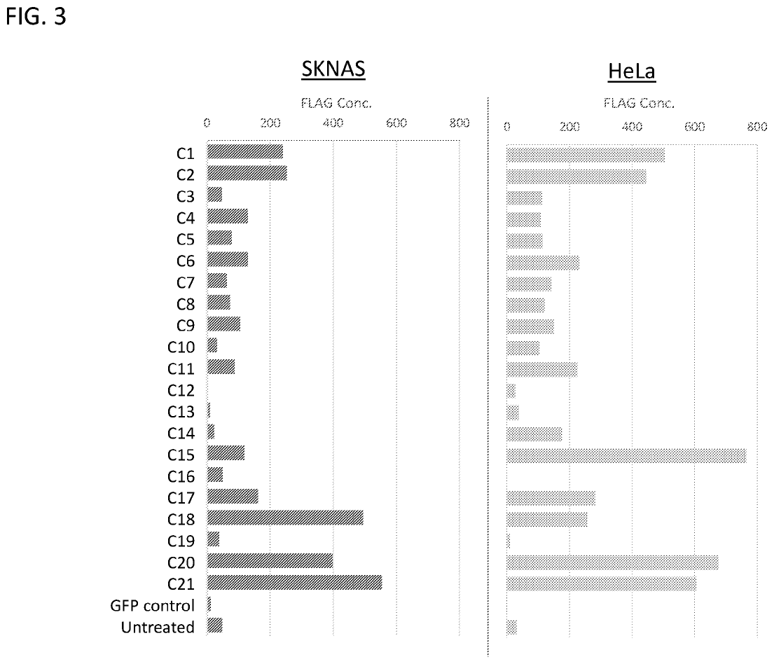

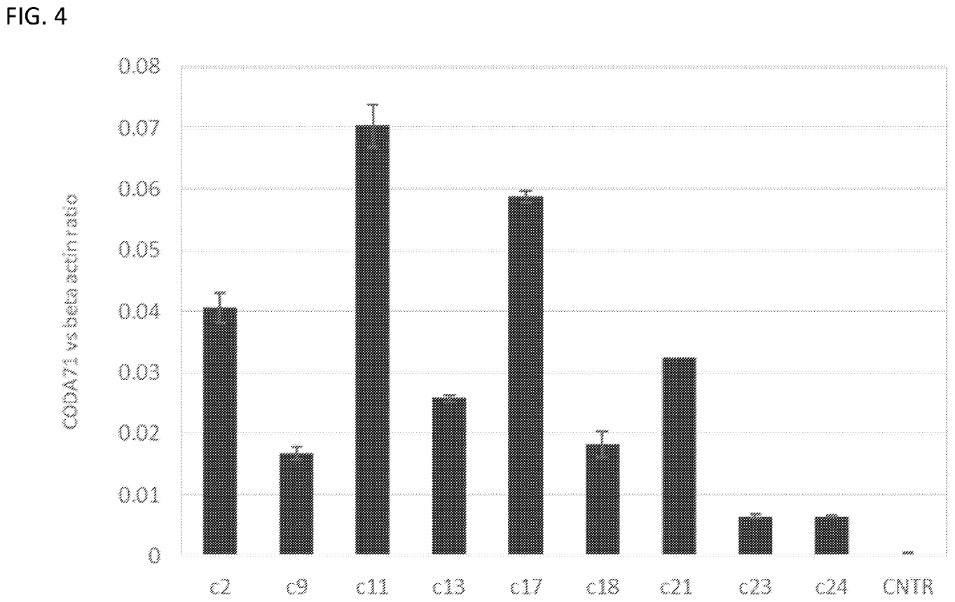

The first round of selection was performed in vitro with 23 unique expression cassettes containing various combinations of regulatory elements including enhancer, promoter, intron, 5′ untranslated region, 3′ untranslated region, and/or polyA tail (FIG. 1). Plasmids with ITRs-flanking the expression cassettes driving LGIC expression were transfected in SKNAS cells, a neuroblastoma cell line, as well as HeLa cells to determine which cassettes were able to target the cells more efficiently and provide high levels of LGIC expression. Analysis by ddPCR (FIG. 2) and ELISA (FIG. 3) showed varying levels of expression, with the highest observed in cassettes containing the CMV promoter followed by those with various tissue-specific promoters. For the second round of selection, 9 expression cassettes chosen from the first round were packaged into AAV9 vectors and transduced in neonatal rat hippocampal mixed cultures. Analysis by ddPCR (FIG. 4) revealed high levels of LGIC mRNA expression from CaMKII- and Syn-driven cassettes, which were comparable to the strong, ubiquitous CAG promoter.

### Example 2: In Vivo Assessment of Expression Cassettes with Optimal Hippocampal Neuron Expression for the Treatment of Focal Epilepsy

ddPCR assessment: Rat hippocampal tissues were homogenized using Pink RINO RNA Lysis Kit. RNA was isolated using the manufacturer's protocol for the RNeasy Plus Mini Kit and PureLink™ DNase. cDNA was synthesized using SuperScript™ IV VILO™ Master Mix with ezDNase™ Enzyme kit. ddPCR reactions were carried with customs primers purchased from Integrated DNA Technologies, TaqMan™ probes from Thermo Fisher Scientific, and the rest of the reagents and instruments from Bio-Rad. The expression of the transgene was compared to a house-keeping gene in same reaction and presented as a ratio of the two.

Immunofluorescence assessment: Whole brains were perfused and fixed in 4% PFA. Immunofluorescence staining was performed to observe FLAG tag expression in the hippocampi.

Data analysis/interpretation: mRNA analysis using ddPCR and immunofluorescence to determine cellular tropism demonstrated high levels of expression from human versions of the Syn (C#11) and CaMKII-driven (C#13) cassettes.

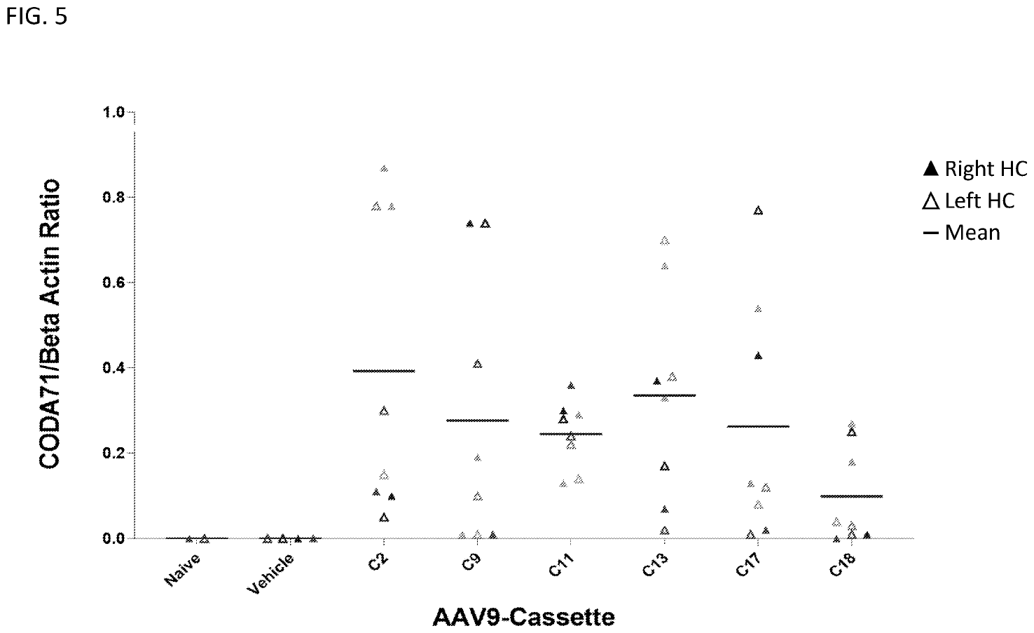

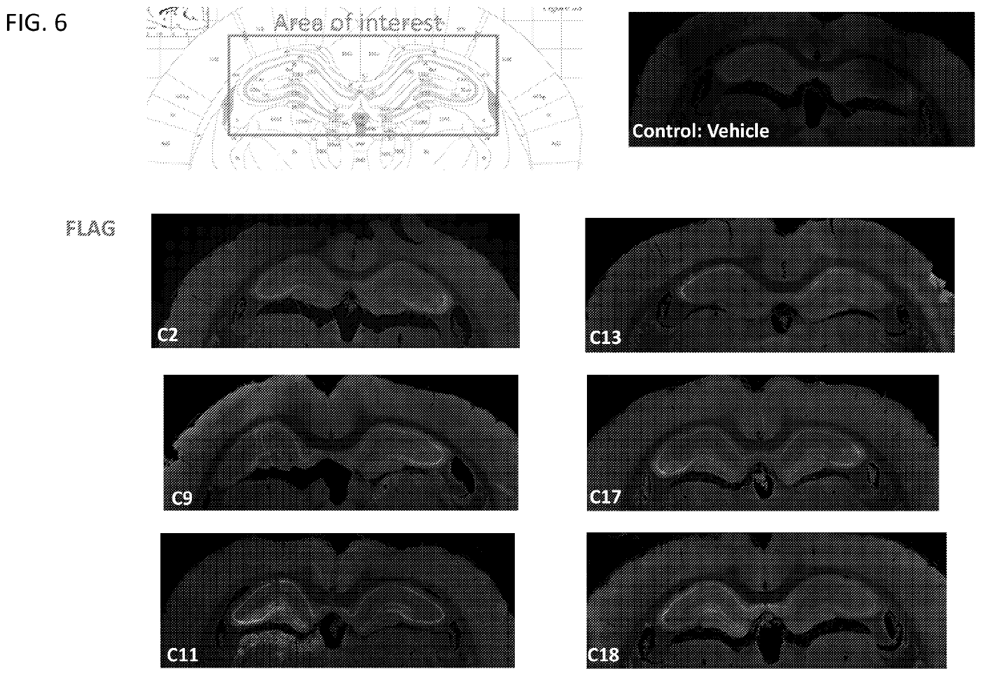

Description of Experiments: The highest expressing cassettes from the second round of selection in the last Example were then assessed in vivo in male Sprague Dawley rat hippocampi by AAV9-mediated directed injection. mRNA analysis using ddPCR (FIG. 5) and immunofluorescence (FIG. 6) for cellular tropism demonstrated high levels of expression from human versions of the Syn- and CaMKII-driven cassettes. Further assessment of the two selected cassettes, one driven by the pan-neuronal hSyn promoter (Expression Cassette #11 in Table 2) and the other by an excitatory cell-specific promoter CaMKII (Expression Cassette #13 in Table 2), will be performed in an animal model of focal epilepsy to finalize the development candidate.

### Example 3: In Vitro Studies for Treating Focal Epilepsy Using Repeated Single Doses of Non-Native Receptor Ligand

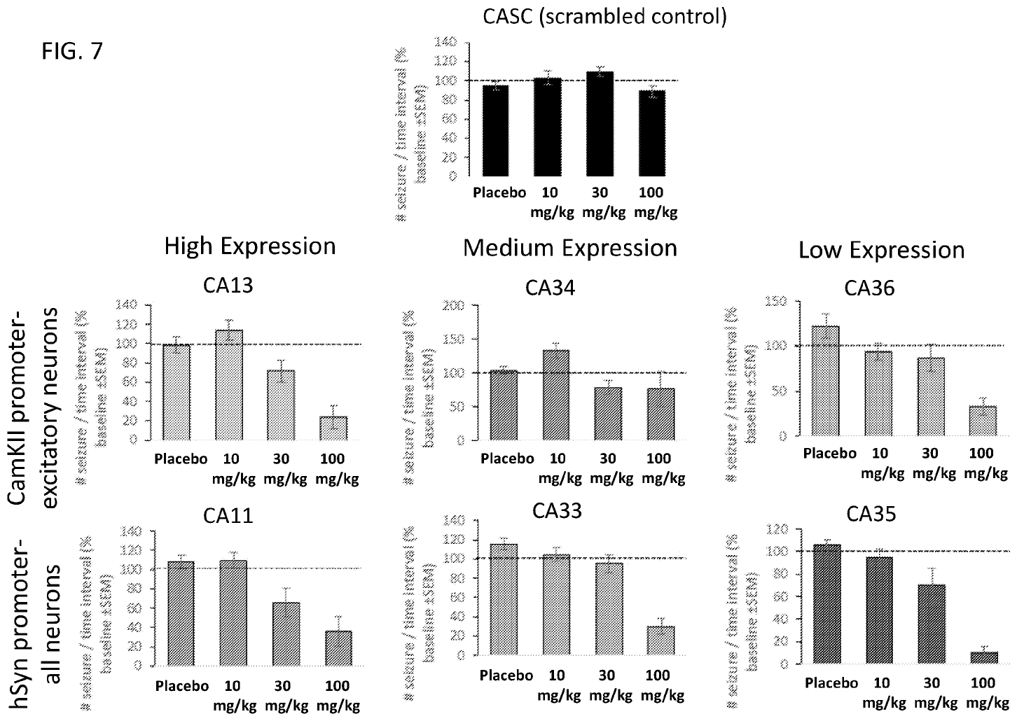

Six expression cassettes—#11, #13, #33, #34, #35, and #36 in Table 2—were each packaged into the AAV vector and tested in a kainic acid (KA) focal epilepsy mouse model. The animals were then monitored for seizure frequency following repeated dosing of TC-5619, an activator of the CODA71 receptor transgene encoded by these expression cassettes. The results (FIG. 7) show that repeated dosing of TC-5619 resulted in a dose-dependent reduction of seizure frequency, and higher dose ranges of TC-5619 significantly reduced seizure burden. In addition, repeated single doses of TC-5619 followed by washout returned all animals to same level of baseline, indicating that the drug had no residual effect. Therefore, this study demonstrates that AAV-delivery of these expression cassettes, in combination with the corresponding receptor activator, can successfully control focal epilepsy in vivo.

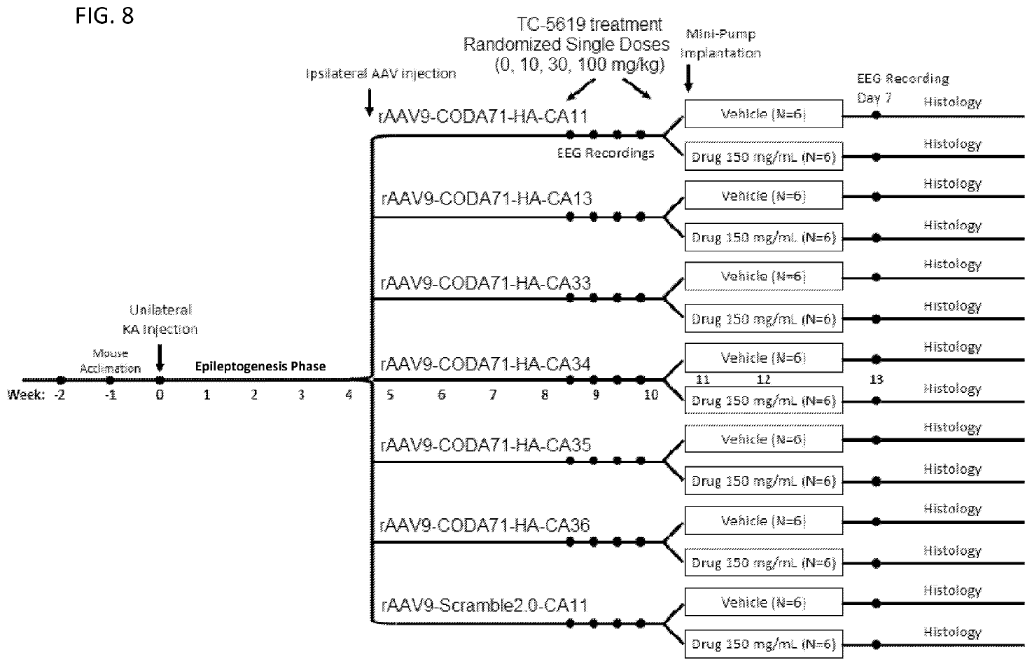

Further efficacy studies will be conducted in the KA focal epilepsy model according to FIG. 8. AAV comprising one of these six CODA71-encoding expression cassettes will be administered four weeks following the kainic acid (KA) administration via stereotactic injection of a 1 uL bolus at the KA lesion site and recording depth electrode will be implanted. An additional four weeks will be allowed for maximal receptor expression prior to the animals being dosed with TC-5619 in a randomized cross over design to assess seizure reduction at different single doses of drug, which will then be followed by chronic administration of TC-5619 or placebo for 7 days via minipump. Histological analysis of receptor expression and cell health measurements will also be performed.

### Example 4: In Vitro and In Vivo Assessment of Expression Cassettes with Optimal DRG Sensory Neuron Expression for the Treatment of Neuropathic Pain

The expression cassettes used here have the same numbering as those used in Example 1 above.

Culture and transduction of human iPSC derived sensory neuron progenitors: Cells were thawed and plated at 100,000 cells/cm² in neural plating medium supplemented with 10 μM Y-27632 (ROCK inhibitor). Media was fully exchanged to sensory neuron maintenance medium supplemented with growth factors and treated with Mitomycin C prior to transduction. Cells were transduced with AAV6-carrying different expression cassettes at an MOI of 3E5 and processed 3 to 5 days later for mRNA or protein analysis.

ddPCR assessment of transduced cells from the in vitro study: RNA was isolated from cells with RNeasy Plus Mini Kit and PureLink™ DNase, using the manufacturer's protocol. cDNA was synthesized using SuperScript™ IV VILO™ Master Mix with ezDNase™ Enzyme kit. ddPCR reactions were carried out with customs primers purchased from Integrated DNA Technologies, TaqMan™ probes from Thermo Fisher Scientific, and the rest of the reagents and instruments from Bio-Rad. Expression of the receptor transgene was compared to a house-keeping gene in the same reaction and presented as a ratio of the two.

ELISA assessment in transduced cells from in vitro study: Cell culture samples were processed using a DYKDDDDK-Tag Protein ELISA Kit according to the manufacturer's protocol. Total protein concentrations were determined per sample using a Micro BCA™ Protein Assay Kit following the manufacture's protocol.

ddPCR assessment of directly injected rat L3 and L4 DRGs from in vivo study: Rat L3 & L4 DRG tissues were homogenized using Pink RINO RNA Lysis Kit. RNA was isolated using the manufacturer's protocol for the RNeasy Plus Mini Kit and PureLink™ DNase. cDNA was synthesized using SuperScript™ IV VILO™ Master Mix with ezDNase™ Enzyme kit. ddPCR reactions were carried with customs primers purchased from Integrated DNA Technologies, TaqMan™ probes from Thermo Fisher Scientific, and the rest of the reagents and instruments from Bio-Rad. The expression of the transgene was compared to a house-keeping gene in same reaction and presented as a ratio of the two.

Immunofluorescence assessment in directly injected rat L5 DRGs from in vivo study: L5 DRGs were rinsed in PBS and fixed in 4% PFA. Immunofluorescence staining was performed to observe FLAG tag expression and counterstained with antibodies for sensory neurons (NeuN) in the L5 DRG.

Analysis/interpretation of in vivo study results: mRNA analysis using ddPCR and immunofluorescence to determine cellular tropism demonstrated high levels of expression from the hSyn-driven cassettes (Cassette #3 and Cassette #11).

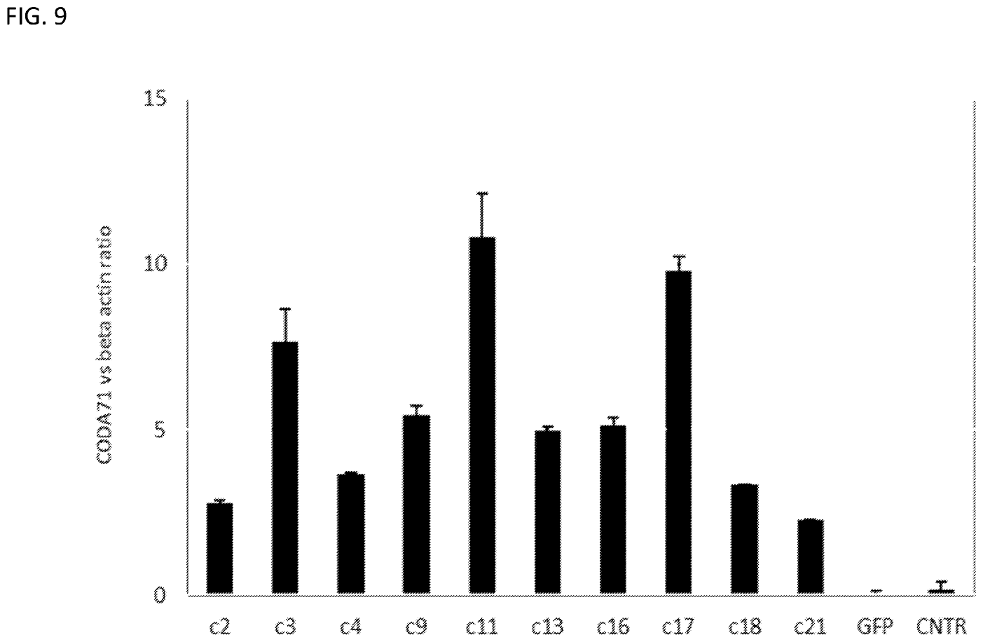

Description of the Experiments: For the in vitro studies, ten expression cassettes chosen from Example 1 above were packaged into AAV6 vectors and transduced human iPSC derived sensory neuron progenitors. ddPCR analysis (FIG. 9) revealed high levels of LGIC mRNA expression from Syn-driven cassettes, which were comparable to the strong, ubiquitous CAG promoter.

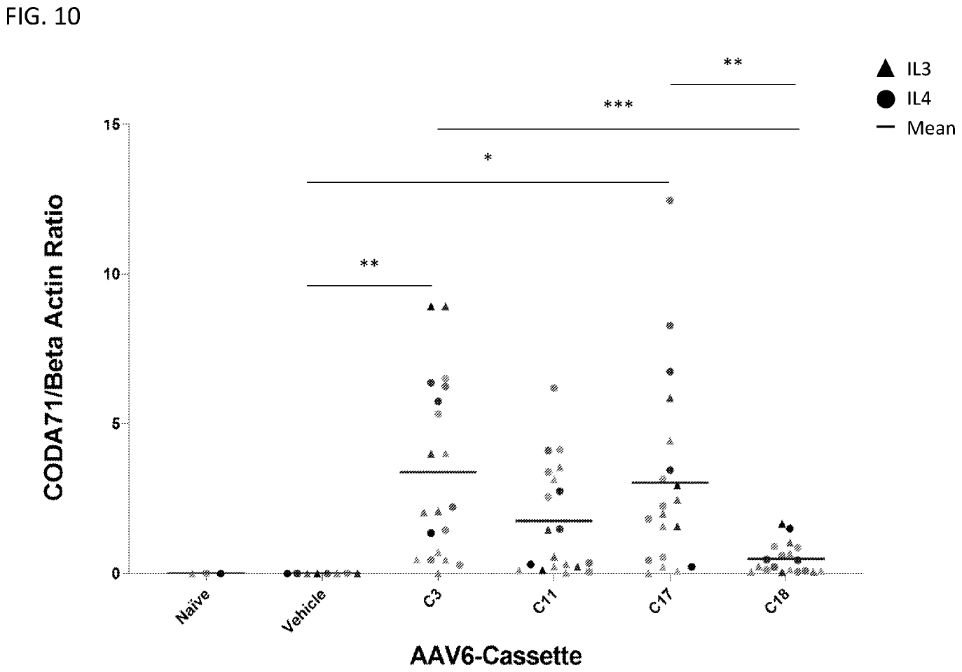

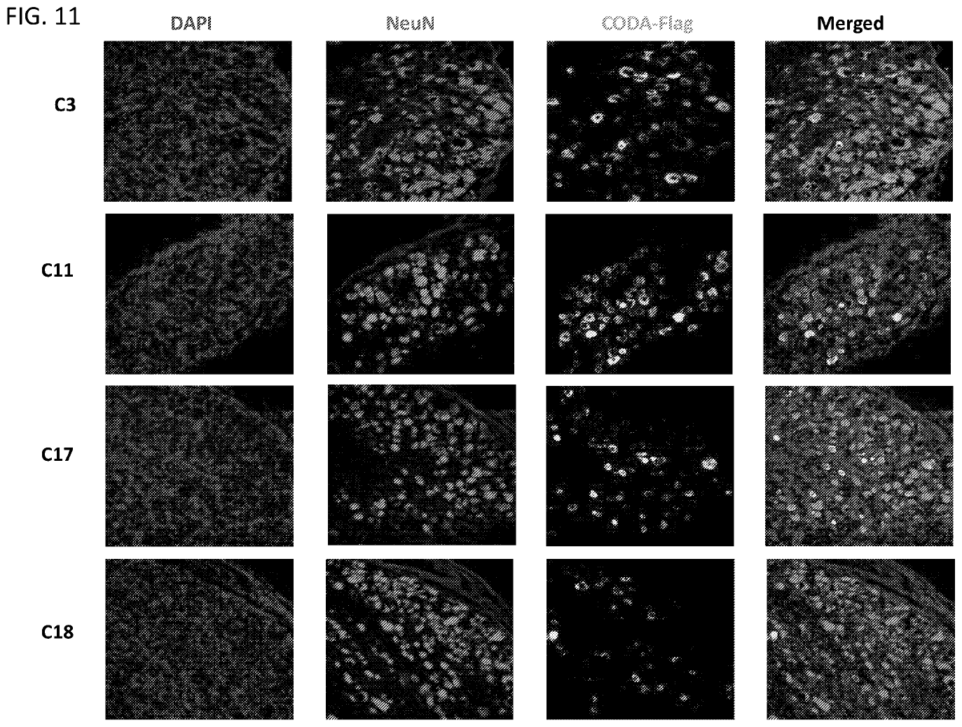

The highest expressing cassettes from the in vitro study were then assessed in vivo in male Sprague Dawley rat DRGs by AAV6-mediated directed injection. mRNA analysis using ddPCR (FIG. 10) and immunofluorescence (FIG. 11) for cellular tropism demonstrated high levels of expression from human versions of the Syn-driven cassettes (Cassettes #3 and #11 in Table 2).

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A recombinant nucleic acid comprising an expression cassette comprising, in 5′ to 3′ order, one or more of a 5′ enhancer, a promoter, a 5′ untranslated region (UTR), a transgene, a 3′ enhancer, and a polyadenylation sequence (polyA), wherein the transgene is operably linked to the promoter.

2. The recombinant nucleic acid of claim 1, wherein the expression cassette comprises the 5′ enhancer comprising a polynucleotide sequence at least 90% identical to any one of SEQ ID NO: 37-39.

3. The recombinant nucleic acid of claim 1, wherein the expression cassette does not comprise the 5′ enhancer.

4. The recombinant nucleic acid of any one of claims 1-3, wherein the expression cassette comprises the promoter comprising a polynucleotide sequence at least 90% identical to any one of SEQ ID NO: 41-51.

5. The recombinant nucleic acid of any one of claims 1-4, wherein the promoter is a neuron-specific promoter.

6. The recombinant nucleic acid of any one of claims 1-5, wherein the expression cassette comprises an intron between the promoter and the transgene.

7. The recombinant nucleic acid of any one of claims 1-5, wherein the expression cassette comprises an intron between the 5′ UTR and the transgene.

8. The recombinant nucleic acid of any one of claims 1-5, wherein the expression cassette comprises an intron between the promoter and the 5′ UTR.

9. The recombinant nucleic acid of any one of claims 6-8, wherein the intron comprises a polynucleotide sequence at least 90% identical to any one of SEQ ID NO: 57-61.

10. The recombinant nucleic acid of any one of claims 1-5, wherein the expression cassette does not comprise an intron.

11. The recombinant nucleic acid of any one of claims 1-10, wherein the expression cassette comprises the 5′ UTR comprising a polynucleotide sequence at least 90% identical to any one of SEQ ID NO: 52-56.

12. The recombinant nucleic acid of any one of claims 1-11, wherein the expression cassette comprises the 3′ enhancer comprising a polynucleotide sequence at least 90% identical to any one of SEQ ID NO: 62-65.

13. The recombinant nucleic acid of any one of claims 1-12, wherein the expression cassette comprises the polyA comprising a polynucleotide sequence at least 90% identical to any one of SEQ ID NO: 67-70.

14. The recombinant nucleic acid of any one of claims 1-13, wherein the expression cassette comprises a non-neuron silencing element embedded in the promoter, and wherein the non-neuron silencing element comprises a polynucleotide sequence at least 90% identical to SEQ ID NO: 40.

15. The recombinant nucleic acid of any one of claims 1-13, wherein the expression cassette comprises a non-neuron silencing element between the 5′ enhancer and the promoter, and wherein the non-neuron silencing element comprises a polynucleotide sequence at least 90% identical to SEQ ID NO: 40.

16. The recombinant nucleic acid of any one of claims 1-15, wherein the expression cassette comprises a 3′ UTR between the 3′ enhancer and the polyA, and wherein the 3′ UTR comprises a polynucleotide sequence at least 90% identical to SEQ ID NO: 66.

17. The recombinant nucleic acid of any one of claims 1-16, wherein the transgene encodes a ligand-gated ion channel (LGIC).

18. The recombinant nucleic acid of claim 17, wherein the ligand-gated ion channel comprises a ligand binding domain derived from human α7 nicotinic acetylcholine receptor (α7-nAChR).

19. The recombinant nucleic acid of claim 18, wherein the ligand binding domain comprises an amino acid sequence having at least 85% identity to amino acid residues 23-220 of SEQ ID NO: 25.

20. The recombinant nucleic acid of claim 18 or 19, wherein the ligand binding domain comprises one or more amino acid mutations listed in Table 5.

21. The recombinant nucleic acid of any one of claims 18-20, wherein ligand-gated ion channel comprises an ion pore domain derived from a human Glycine receptor.

22. The recombinant nucleic acid of claim 21, wherein the ion pore domain comprises an amino acid sequence having at least 85% identity to amino acids 255-457 of SEQ ID NO: 26, 260-452 of SEQ ID NO: 27, amino acids 259-464 of SEQ ID NO: 28, or amino acids 259-449 of SEQ ID NO: 29.

23. The recombinant nucleic acid of claim 21 or 22, wherein the ligand binding domain of the engineered receptor comprises a Cys-loop domain derived from the human Glycine receptor.

24. The recombinant nucleic acid of any one of claims 21-23, wherein the ligand-gated ion channel comprises an amino acid sequence having at least 95% sequence identity to any one of SEQ ID NO: 25-31 and 33.

25. The recombinant nucleic acid of any one of claims 21-23, wherein the human Glycine receptor is human Glycine receptor α1, and wherein the ligand-gated ion channel comprises an amino acid sequence having at least 95% sequence identity to SEQ ID NO: 33.

26. The recombinant nucleic acid of any one of claims 1-25, wherein the transgene comprises or consists of a polynucleotide sequence having at least 90%, at least 95%, at least 96%, at least 97%, at least 98%, at least 99%, at least 99.5%, or 100% identity to SEQ ID NO: 32.

27. The recombinant nucleic acid of any one of claims 1-26, wherein the transgene is codon-optimized for expression in a human cell.

28. The recombinant nucleic acid of claim 27, wherein the human cell is a neuron.

29. The recombinant nucleic acid of any one of claims 1-28, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(iii) a hCaMKII 5′ UTR comprising a polynucleotide sequence having at least 90%, at least 95%, at least 96%, at least 97%, at least 98%, at least 99%, at least 99.5%, or 100% identity to SEQ ID NO: 53;
(iv) a hCMV+rGlob Intron comprising a polynucleotide sequence having at least 90%, at least 95%, at least 96%, at least 97%, at least 98%, at least 99%, at least 99.5%, or 100% identity to SEQ ID NO: 58;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68.

30. The recombinant nucleic acid of claim 29, wherein the expression cassette comprises, in 5′ to 3′ order, a sequence having at least 90%, at least 95%, at least 96%, at least 97%, at least 98%, at least 99%, at least 99.5%, or 100% identity to nucleotides 173-1877 of SEQ ID NO: 10⁸, the transgene, and a sequence having at least 90%, at least 95%, at least 96%, at least 97%, at least 98%, at least 99%, at least 99.5%, or 100% identity to nucleotides 3237-4315 of SEQ ID NO: 10⁸.

31. The recombinant nucleic acid of any one of claims 1-28, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hSyn intron comprising a polynucleotide sequence having at least 90%, at least 95%, at least 96%, at least 97%, at least 98%, at least 99%, at least 99.5%, or 100% identity to SEQ ID NO: 59;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68.

32. The recombinant nucleic acid of claim 31, wherein the expression cassette comprises, in 5′ to 3′ order, a sequence having at least 90%, at least 95%, at least 96%, at least 97%, at least 98%, at least 99%, at least 99.5%, or 100% identity to nucleotides 173-1159 of SEQ ID NO: 106, the transgene, and a sequence having at least 90%, at least 95%, at least 96%, at least 97%, at least 98%, at least 99%, at least 99.5%, or 100% identity to nucleotides 2519-3597 of SEQ ID NO: 106.

33. The recombinant nucleic acid of any one of claims 1-28, wherein the expression cassette comprises, in 5′ to 3′ order,
(i) a CMV-V2 5′ enhancer comprising a polynucleotide sequence having at least 90%, at least 95%, at least 96%, at least 97%, at least 98%, at least 99%, at least 99.5%, or 100% identity to SEQ ID NO: 38;
(ii) a hSyn-V2 promoter comprising a polynucleotide sequence having at least 90%, at least 95%, at least 96%, at least 97%, at least 98%, at least 99%, at least 99.5%, or 100% identity to SEQ ID NO: 49;
(iii) the transgene;
(iv) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(v) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

34. The recombinant nucleic acid of claim 33, wherein the expression cassette comprises, in 5′ to 3′ order, a sequence having at least 90%, at least 95%, at least 96%, at least 97%, at least 98%, at least 99%, at least 99.5%, or 100% identity to nucleotides 149-946 of SEQ ID NO: 125, the transgene, and a sequence having at least 90%, at least 95%, at least 96%, at least 97%, at least 98%, at least 99%, at least 99.5%, or 100% identity to nucleotides 2285-3416 of SEQ ID NO: 125.

35. The recombinant nucleic acid of any one of claims 1-28, wherein the expression cassette comprises, in 5′ to 3′ order,
(i) a CMV-V2 5′ enhancer comprising a polynucleotide sequence having at least 90%, at least 95%, at least 96%, at least 97%, at least 98%, at least 99%, at least 99.5%, or 100% identity to SEQ ID NO: 38;
(ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(iii) the transgene;
(iv) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(v) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

36. The recombinant nucleic acid of claim 35, wherein the expression cassette comprises, in 5′ to 3′ order, a sequence having at least 90%, at least 95%, at least 96%, at least 97%, at least 98%, at least 99%, at least 99.5%, or 100% identity to nucleotides 149-1527 of SEQ ID NO: 126, the transgene, and a sequence having at least 90%, at least 95%, at least 96%, at least 97%, at least 98%, at least 99%, at least 99.5%, or 100% identity to nucleotides 2848-3979 of SEQ ID NO: 126.

37. The recombinant nucleic acid of any one of claims 1-28, wherein the expression cassette comprises, in 5′ to 3′ order,
(i) a hSyn-V2 promoter comprising a polynucleotide sequence having at least 90%, at least 95%, at least 96%, at least 97%, at least 98%, at least 99%, at least 99.5%, or 100% identity to SEQ ID NO: 49;
(ii) the transgene;
(iii) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(iv) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

38. The recombinant nucleic acid of claim 37, wherein the expression cassette comprises, in 5′ to 3′ order, a sequence having at least 90%, at least 95%, at least 96%, at least 97%, at least 98%, at least 99%, at least 99.5%, or 100% identity to nucleotides 153-661 of SEQ ID NO: 127, the transgene, and a sequence having at least 90%, at least 95%, at least 96%, at least 97%, at least 98%, at least 99%, at least 99.5%, or 100% identity to nucleotides 1982-3113 of SEQ ID NO: 127.

39. The recombinant nucleic acid of any one of claims 1-28, wherein the expression cassette comprises, in 5′ to 3′ order,
(i) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(ii) the transgene;
(iii) a WPREx-V2 3′ enhancer comprising a polynucleotide sequence having at least 90%, at least 95%, at least 96%, at least 97%, at least 98%, at least 99%, at least 99.5%, or 100% identity to SEQ ID NO: 65; and
(iv) a hGH-V2 polyA comprising a polynucleotide sequence having at least 90%, at least 95%, at least 96%, at least 97%, at least 98%, at least 99%, at least 99.5%, or 100% identity to SEQ ID NO: 69.

40. The recombinant nucleic acid of claim 39, wherein the expression cassette comprises, in 5′ to 3′ order, a sequence having at least 90%, at least 95%, at least 96%, at least 97%, at least 98%, at least 99%, at least 99.5%, or 100% identity to nucleotides 153-1224 of SEQ ID NO: 128, the transgene, and a sequence having at least 90%, at least 95%, at least 96%, at least 97%, at least 98%, at least 99%, at least 99.5%, or 100% identity to nucleotides 2545-3676 of SEQ ID NO: 128.

41. The recombinant nucleic acid of any one of claims 1-28, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hTPI intron comprising a polynucleotide sequence having at least 90%, at least 95%, at least 96%, at least 97%, at least 98%, at least 99%, at least 99.5%, or 100% identity to SEQ ID NO: 60;
(v) the transgene;
(vi) a FullEES 3′ enhancer comprising a polynucleotide sequence having at least 90%, at least 95%, at least 96%, at least 97%, at least 98%, at least 99%, at least 99.5%, or 100% identity to SEQ ID NO: 63; and
(vii) a rβGlobin polyA comprising a polynucleotide sequence having at least 90%, at least 95%, at least 96%, at least 97%, at least 98%, at least 99%, at least 99.5%, or 100% identity to SEQ ID NO: 70.

42. The recombinant nucleic acid of claim 41, wherein the expression cassette comprises, in 5′ to 3′ order, a sequence having at least 90%, at least 95%, at least 96%, at least 97%, at least 98%, at least 99%, at least 99.5%, or 100% identity to nucleotides 173-1188 of SEQ ID NO: 98, the transgene, and a sequence having at least 90%, at least 95%, at least 96%, at least 97%, at least 98%, at least 99%, at least 99.5%, or 100% identity to nucleotides 2548-3824 of SEQ ID NO: 98.

43. The recombinant nucleic acid of any one of claims 1-42, wherein the expression cassette comprises (i) a polynucleotide sequence having at least 90% identity to any one of SEQ ID NO: 71-93 excluding the sequence of the transgene (SEQ ID NO: 36), or (ii) a polynucleotide sequence having at least 90% identity to any one of SEQ ID NO: 121-124 excluding the sequence of the transgene (SEQ ID NO: 32).

44. The recombinant nucleic acid of any one of claims 1-42, wherein the expression cassette comprises a polynucleotide sequence having at least 90% identity to any one of SEQ ID NO: 71-93 and 121-124.

45. The recombinant nucleic acid of any one of claims 1-44, comprising an adeno-associated virus (AAV) inverted terminal repeat (ITR) flanking each end of the expression cassette.

46. The recombinant nucleic acid of claim 45, comprising a 5′ ITR sequence having at least 90% identity to SEQ ID NO: 94 or 119 and a 3′ ITR sequence having at least 90% identity to SEQ ID NO: 95 or 120.

47. The recombinant nucleic acid of any one of claims 1-45, comprising (i) a polynucleotide sequence having at least 90% identity to any one of SEQ ID NO: 96-118 excluding the sequence of the transgene (SEQ ID NO: 36), or (ii) a polynucleotide sequence having at least 90% identity to any one of SEQ ID NO: 125-128 excluding the sequence of the transgene (SEQ ID NO: 32).

48. The recombinant nucleic acid of any one of claims 1-45, comprising a polynucleotide sequence having at least 90% identity to any one of SEQ ID NO: 96-118 and 125-128.

49. A vector comprising the recombinant nucleic acid of any one of claims 1-48.

50. The vector of claim 49, wherein the vector is a non-viral vector.

51. The vector of claim 49, wherein the vector is a viral vector.

52. The vector of claim 51, wherein the vector comprises or consists of an AAV vector genome.

53. An AAV comprising the vector of claim 51 or 52.

54. The AAV of claim 53, wherein the AAV is AAV9 serotype.

55. The AAV of claim 53 or 54, wherein the AAV is a self-complementary AAV or a single stranded AAV.

56. The AAV of any one of claims 53-55, wherein the AAV is a wild-type AAV or a modified AAV.

57. The AAV of any one of claims 53-56, wherein the AAV comprises a capsid protein having at least 95% identity to an AAV9 capsid protein (SEQ ID NO: 8) or AAV9-TV capsid protein (SEQ ID NO: 9).

58. A host cell, comprising the nucleic acid of any one of claims 1-48, or the vector of any one of claims 49-52.

59. A method of producing the AAV of any one of claims 53-57.

60. A kit comprising the recombinant nucleic acid of any one of claims 1-48, the vector of any one of claims 49-52, or the AAV of any one of claims 53-57.

61. A method of expressing a transgene in a cell, comprising delivering the recombinant nucleic acid of any one of claims 1-48 or the vector of any one of claims 49-52 to the cell.

62. A method of transducing a cell, comprises contacting the cell with the AAV of any one of claims 53-57.

63. The method of claim 61 or 62, wherein the cell is a neuron.

64. The method of claim 63, wherein the neuron is a hippocampal neuron.

65. The method of claim 64, wherein the neuron is an excitatory neuron.

66. The method of claim 65, wherein the neuron is a CAMK2 positive neuron.

67. The method of claim 64, wherein the neuron is an inhibitory neuron.

68. The method of claim 67, wherein the neuron is a GABAergic neuron.

69. The method of claim 63, wherein the neuron is a dorsal root ganglion neuron or a trigeminal ganglion neuron.

70. The method of claim 69, wherein the neuron comprises an isolectin B4 (IB4) positive nerve fiber.

71. The method of claim 69 or 70, wherein the neuron comprises an NF200 positive nerve fiber.

72. The method of any one of claims 69-71, wherein the neuron comprises a CGRP positive nerve fiber.

73. The method of any one of claims 69-72, wherein the neuron comprises a C fiber.

74. The method of any one of claims 69-73, wherein the neuron comprises an Aδ fiber.

75. The method of any one of claims 61-74, wherein the cell is an ex vivo cell.

76. The method of any one of claims 61-74, wherein the cell is an in vivo cell of a subject, optionally wherein the subject is a human.

77. The method of any one of claims 61-76, wherein the cell comprising the expression cassette has a higher expression level of the transgene compared to a corresponding cell comprising a control expression cassette, optionally wherein said higher expression is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least ten-fold higher than the transgene expression level of the control expression cassette comprising the polynucleotide sequence of SEQ ID NO: 88 excluding the transgene sequence.

78. The method of any one of claims 61-76, wherein the cell comprising the expression cassette has a comparable or higher expression level of the transgene compared to a corresponding cell comprising a control expression cassette, optionally wherein said transgene expression level is at least 5%, at least 10%, at least 20%, at least 30%, at least 40%, at least 50%, at least 60%, at least 70%, at least 80%, at least 90%, at least two-fold, at least three-fold, at least four-fold, at least five-fold, or at least ten-fold of the transgene expression level of the control expression cassette comprising the polynucleotide sequence of SEQ ID NO: 87 excluding the transgene sequence.

79. A method of treating a disease or disorder in a subject in need thereof, comprises administering an effective amount of the recombinant nucleic acid of any one of claims 1-48, the vector of any one of claims 49-52, or the AAV of any one of claims 53-57 to the subject.

80. The method of claim 79, wherein the disease or disorder is epilepsy, schizophrenia, autism spectrum disorder, Alzheimer's disease, Rett syndrome, or fragile X syndrome.

81. The method of claim 79, wherein the disease or disorder is focal epilepsy.

82. The method of claim 79, wherein the disease or disorder is mesial temporal lobe epilepsy (mTLE).

83. The method of any one of claims 61-82, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hCaMKIIa promoter comprising a polynucleotide sequence having at least 90%, at least 95%, at least 96%, at least 97%, at least 98%, at least 99%, at least 99.5%, or 100% identity to SEQ ID NO: 43;
(iii) a hCaMKII 5′ UTR comprising a polynucleotide sequence having at least 90%, at least 95%, at least 96%, at least 97%, at least 98%, at least 99%, at least 99.5%, or 100% identity to SEQ ID NO: 53;
(iv) a hCMV+rGlob Intron comprising a polynucleotide sequence having at least 90%, at least 95%, at least 96%, at least 97%, at least 98%, at least 99%, at least 99.5%, or 100% identity to SEQ ID NO: 58;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68,
optionally, wherein the transgene encodes a ligand-gated ion channel comprising a ligand binding domain derived from human α7-nAChR and an ion pore domain derived from a human Glycine receptor.

84. The method of any one of claims 61-82, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hSyn intron comprising a polynucleotide sequence having at least 90%, at least 95%, at least 96%, at least 97%, at least 98%, at least 99%, at least 99.5%, or 100% identity to SEQ ID NO: 59;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68,
optionally, the transgene encodes a ligand-gated ion channel comprising a ligand binding domain derived from human α7-nAChR and an ion pore domain derived from a human Glycine receptor.

85. The method of any one of claims 79-84, wherein the recombinant nucleic acid, the vector, or the AAV is administered by intracranial administration, intrathecal (spine) administration, intrathecal (Cisterna magna) administration, intracerebral administration, intraventricular administration, or direct injection into the epileptic focus in hippocampus.

86. The method of any one of claims 79-84, wherein the recombinant nucleic acid, the vector, or the AAV is administered by direct injection into the epileptic focus in hippocampus.

87. The method of any one of claims 79-86, wherein 1×10⁹-1×10¹⁴ copies of the recombinant nucleic acid or AAV vector genome are administered to the subject.

88. The method of any one of claims 79-87, wherein the AAV comprises a capsid protein of AAV9 (SEQ ID NO: 8).

89. The method of any one of claims 79-88, wherein the method decreases the duration, intensity, and/or frequency of the epilepsy by at least 10%.

90. A method of treating a disease or disorder in a subject in need thereof, comprises administering an effective amount of the recombinant nucleic acid of any one of claims 1-48, the vector of any one of claims 49-52, or the AAV of any one of claims 53-57 to the subject, wherein the disease or disorder is neuropathic pain, spasticity, spinal cord injury, or avulsion injury.

91. The method of claim 90, wherein the disease or disorder is neuropathic pain.

92. The method of claim 91, wherein the neuropathic pain is peripheral neuropathy.

93. The method of claim 91, wherein the neuropathic pain is trigeminal neuralgia.

94. The method of any one of claims 61-78 and 90-93, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hTPI intron comprising a polynucleotide sequence having at least 90%, at least 95%, at least 96%, at least 97%, at least 98%, at least 99%, at least 99.5%, or 100% identity to SEQ ID NO: 60;
(v) the transgene;
(vi) a FullEES 3′ enhancer comprising a polynucleotide sequence having at least 90%, at least 95%, at least 96%, at least 97%, at least 98%, at least 99%, at least 99.5%, or 100% identity to SEQ ID NO: 63; and
(vii) a rβGlobin polyA comprising a polynucleotide sequence having at least 90%, at least 95%, at least 96%, at least 97%, at least 98%, at least 99%, at least 99.5%, or 100% identity to SEQ ID NO: 70,
optionally, the transgene encodes a ligand-gated ion channel comprising a ligand binding domain derived from human α7-nAChR and an ion pore domain derived from a human Glycine receptor.

95. The method of any one of claims 61-78 and 90-93, wherein the expression cassette comprises, in 5′ to 3′ order,
(i) a CMV 5′ enhancer comprising a polynucleotide sequence having at least 90%, at least 95%, at least 96%, at least 97%, at least 98%, at least 99%, at least 99.5%, or 100% identity to SEQ ID NO: 37;
(ii) a hSyn promoter comprising a polynucleotide sequence having at least 90%, at least 95%, at least 96%, at least 97%, at least 98%, at least 99%, at least 99.5%, or 100% identity to SEQ ID NO: 48;
(iii) a hSyn 5′ UTR comprising a polynucleotide sequence having at least 90%, at least 95%, at least 96%, at least 97%, at least 98%, at least 99%, at least 99.5%, or 100% identity to SEQ ID NO: 54;
(iv) a hSyn intron comprising a polynucleotide sequence having at least 90%, at least 95%, at least 96%, at least 97%, at least 98%, at least 99%, at least 99.5%, or 100% identity to SEQ ID NO: 59;
(v) the transgene;
(vi) a WPREx 3′ enhancer comprising a polynucleotide sequence having at least 90%, at least 95%, at least 96%, at least 97%, at least 98%, at least 99%, at least 99.5%, or 100% identity to SEQ ID NO: 64;
(vii) an αGlobin 3′ UTR comprising a polynucleotide sequence having at least 90%, at least 95%, at least 96%, at least 97%, at least 98%, at least 99%, at least 99.5%, or 100% identity to SEQ ID NO: 66; and
(viii) a hGH polyA comprising a polynucleotide sequence having at least 90%, at least 95%, at least 96%, at least 97%, at least 98%, at least 99%, at least 99.5%, or 100% identity to SEQ ID NO: 68,
optionally, the transgene encodes a ligand-gated ion channel comprising a ligand binding domain derived from human α7-nAChR and an ion pore domain derived from a human Glycine receptor.

96. The method of any one of claims 90-95, wherein the recombinant nucleic acid, the vector, or the AAV is administered by intrathecal (IT) or intraganglionic (IG) administration.

97. The method of any one of claims 90-95, wherein the recombinant nucleic acid, the vector, or the AAV is administered by intraganglionic (IG) administration directly into dorsal root ganglion or trigeminal ganglion.

98. The method of any one of claims 90-97, wherein 1×10⁹-1×10¹⁴ copies of the recombinant nucleic acid or AAV vector genome are administered to the subject.

99. The method of any one of claims 90-98, wherein the AAV comprises a capsid protein of AAV9-TV (SEQ ID NO: 9).

100. The method of any one of claims 91-99, wherein the method lowers the level of the pain by at least 10%.

101. The method of any one of claims 79-100, wherein the recombinant nucleic acid, the vector, or the AAV is administered by systemic, parenteral, intravenous, cerebral, cerebrospinal, intrathecal, intracisternal, intraputaminal, intrahippocampal, intra-striatal, or intra-cerebroventricular injection.

102. The method of any one of claims 79-101, wherein 1×10⁹-1×10¹⁴ copies of the recombinant nucleic acid or AAV vector genome are administered to the subject.

103. The method of any one of claims 79-102, wherein the method comprises administering a ligand of a ligand-gated ion channel encoded by the transgene.

104. The method of claim 103, wherein the ligand is selected from the group consisting of AZD-0328, TC-6987, ABT-126, TC-5619, TC-6683, Varenicline, and Facinicline/RG3487.
